(19) United States
(12) Patent Application Publication
(10) Pub. No.: US 2007/0060582 A1
(43) Pub. Date: Mar. 15, 2007

(54) 1,4-Disubstituted isoquinilone derivatives as raf-kinase inhibitors useful for the treatment of proliferative diseases

(76) Inventors: Cynthia Anne Fink, Lebanon, NJ (US); Lawrence Blas Perez, Hopkinton, MA (US); Timothy Michael Ramsey, Weston, MA (US); Naeem Yusuff, Cambridge, MA (US); Richard William Versace, Wanaque, NJ (US); David Bryant Batt, Wayland, MA (US); Michael Lloyd Sabio, Ridgewood, NJ (US); Sunkyu Kim, Cambridge, MA (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/573,208

(22) PCT Filed: Sep. 23, 2004
(86) PCT No.: PCT/EP04/10688

§ 371 Date: Nov. 17, 2006

Related U.S. Application Data
(60) Provisional application No. 60/505,457, filed on Sep. 24, 2003.

(51) Int. Cl. A61K 31/5377; A61K 31/496; A61K 31/4709; C07D 413/02; C07D 403/02

(52) U.S. Cl. 514/232.8; 514/253.05; 514/256; 514/310; 544/333; 544/363; 546/148

(57) ABSTRACT

This invention relates to compounds of the formula (I) 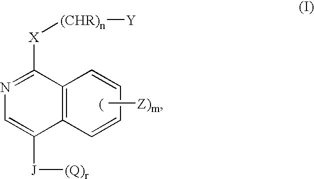 wherein the variable substituents are described herein. The compounds are useful for the treatment of conditions and diseases characterized by an aberrant MAP kinase signaling pathway, such as cancer.

### BACKGROUND OF THE INVENTION

The present invention relates to the discovery of novel compounds that inhibit B-RAF kinase, a serine/threonine kinase that functions in the MAP kinase signaling pathway, and to the use of the compounds for the treatment of diseases characterized by an aberrant MAP kinase signaling pathway, e.g., proliferative diseases like certain cancers.

### SUMMARY OF THE INVENTION

Cells communicate various aspects of their extracellular environment to the nucleus by using various signal transduction pathways. Many of these signals are transmitted by protein kinases which activate various factors through the transfer of phosphate groups. Disruption of signal transduction by inhibiting appropriate kinase activity can have a clinical benefit as has been demonstrated by imatinib, an inhibitor of bcr-abl kinase, which is marketed as its mesylate salt under the brand GLEEVEC™ (in the United States) or GLIVEC®.

Many growth factors send their signal to proliferate from the extracellular environment to the cell nucleus via the MAP kinase signaling pathway. The growth factors activate transmembrane receptors located on the cell surface which in turn start a cascade whereby RAS is activated and recruits RAF kinase to the membrane where it is activated and in turn activates MEK kinase which then activates ERK kinase. Activated ERK kinase can move to the nucleus where it activates various gene transcription factors. Aberrations in this pathway can lead to altered gene transcription, cellular growth and contribute to tumorogenicity by negatively regulating apoptosis and transmitting proliferative and angiogenic signals. Inhibitors of RAF kinase have been shown to block signaling through the MAP kinase signaling pathway in cell culture.

The RAF kinase family is known to have three members designated C-RAF, also known as RAF-1, B-RAF and A-RAF. It has been reported that B-RAF kinase is commonly activated by one of several somatic point mutations in human cancer, including 59% of the melanoma cell lines tested. See Davies et al., Nature, Vol. 417, pp. 949-954 (2002). The compounds described herein are efficient inhibitors of RAF kinase, particularly C-RAF kinase and wild and mutated B-RAF kinase, particularly the V599E mutant B-RAF kinase.

The RAF kinase inhibiting property of the inventive compounds makes them useful as therapeutic agents for the treatment for proliferative diseases characterized by an aberrant MAP kinase signaling pathway, particularly melanoma and other cancer having mutated B-RAF, especially wherein the mutated B-RAF is the V599E mutant. The present invention also provides a method of treating other conditions characterized by mutant B-RAF, e.g., benign Nevi moles having mutated B-RAF, with the isoquinoline compounds.

### DESCRIPTION OF THE INVENTION

The present invention relates compounds of the formula (I) 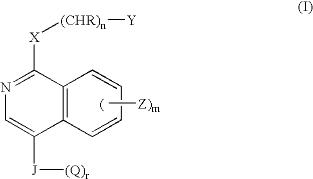
wherein
n is from 0-2;
r is from 0 to 2
m is from 0-4;
J is unsubstituted or substituted once or twice by Q, wherein
J is aryl, heteroaryl, cycloalkyl or heterocycloalkyl, wherein
   aryl is an aromatic radical having from 6-14 carbon atoms, such as phenyl, naphthyl, fluorenyl and phenanthrenyl;
   heteroaryl is an aromatic radical having from 4-14, especially from 5-7 ring atoms, of which 1, 2 or 3 atoms are chosen independently from N, S and O, such as furyl, pyranyl, pyridyl, 1,2-, 1,3- and 1,4-pyrimidinyl, pyrazinyl, triazinyl, triazolyl, oxazolyl, quinazolyl, imidazolyl, pyrrolyl, isoxazolyl isothiazolyl, indolyl, isoindolinyl, quinolyl, isoquinolyl, purinyl, cinnolinyl, naphthyridinyl, phthalazinyl, isobenzofuranyl, chromenyl, purinyl, thianthrenyl, xanthenyl, acridinyl, carbazolyl and phenazinyl;
   cycloalkyl is a saturated cyclic radical having from 3-8, preferably from 5-6 ring atoms, such as cyclopropyl, cyclopentyl and cyclohexyl;
   heterocycloalkyl is a saturated cyclic radical having from 3-8, preferably from 5-6 ring atoms, of which 1, 2 or 3 atoms are chosen independently from N, S and O, such as piperidyl, piperazinyl, imidazolidinyl, pyrrolidinyl and pyrazolidinyl;
Q is a substituent on 1 or 2 carbon atoms selected from the group consisting of halogen, unsubstituted or substituted lower alkyl, —OR₂, —SR₂, —N(R)R, —NRS(O)₂N(R)R, —NRS(O)₂R, —S(O)R₂, —S(O)₂R₂, —OCOR₂, —C(O)R₂, —CO₂R₂, —NR—COR₂, —CON(R₂)R₂, —S(O)₂N(R₂)R₂, cyano, tri-methylsilanyl, unsubstituted or substituted aryl, unsubstituted or substituted heteroaryl, such as substituted or unsubstituted imidazolyl, and substituted or unsubstituted pyridinyl, unsubstituted or substituted cycloalkyl, unsubstituted or substituted heterocycloalkyl, such as substituted or unsubstituted piperidinyl, substituted or unsubstituted piperazolyl, substituted or unsubstituted tetrahydropyranyl, and substituted or unsubstituted azetidinyl, —C₁₋₄alkyl-aryl, —C₁₋₄alkyl-heteroaryl, —C₁₋₄alkyl-heterocyclyl, amino, mono- or di-substituted amino, heteroaryl-aryl;
R is H, lower alkyl or lower alkoxy-alkyl;
R₂ is unsubstituted or substituted alkyl, unsubstituted or substituted cycloalkyl, unsubstituted or substituted phenyl, —C₁₋₄alkyl-aryl, —C₁₋₄alkyl-heteroaryl or —C₁₋₄alkyl-heterocycloalkyl;
X is a bond, Y, —N(R)—, oxa, thio, sulfone, sulfoxide, sulfonamide, amide, or ureylene, preferably —NH—, —NHC(O)—, —NHC(O)NH—;
Y is H, lower alkyl, substituted or unsubstituted aryl, substituted or unsubstituted heteroaryl, substituted or unsubstituted cycloalkyl or substituted or unsubstituted heterocycloalkyl; and
Z is amino, mono- or di-substituted amino, halogen, alkyl, substituted alkyl, hydroxy, etherified or esterified hydroxy, nitro, cyano, carboxy, esterified carboxy, alkanoyl, carbamoyl, N-mono- or N,N-di-substituted carbamoyl, amidino, guanidino, mercapto, sulfo, phenylthio, phenyl-lower alkylthio, alkylphenylthio, phenylsulfinyl, phenyl-lower alkylsulfinyl, alkylphenylsulfinyl, phenylsulfonyl, phenyl-lower alkanesulfonyl or alkylphenylsulfonyl, and where, if more than one radical Z is present (m≧2), the substituents Z are Identical or different;
or an N-oxide of the mentioned compound, wherein one or more N atoms carry an oxygen atom;
   or a pharmaceutically acceptable salt thereof.

The compounds of formula (I) inhibit RAF kinase and have pharmaceutical utility based on this property.

Within the context of the present disclosure, the general terms used hereinbefore and hereinafter preferably have the following meanings, unless indicated otherwise.

The term “lower” denotes a radical having up to and including a maximum of 7, especially up to and including a maximum of 4 carbon atoms, the radicals in question being unbranched or branched one or more times.

Any reference to compounds, salts and the like in the plural is always to be understood as including one compound, one salt or the like.

Asymmetric carbon atoms which may be present, e.g., in compounds of formula (I) (or an N-oxide thereof), wherein n=1 and R is lower alkyl; may have the (R), (S) or (R,S) configuration, preferably the (R) or (S) configuration. Substituents at a double bond or a ring may be in the cis (=Z) or trans (=E) form. Accordingly, the present compounds may be in the form of isomeric mixtures or in the form of pure isomers, preferably in the form of an enantiomerically pure diastereoisomer.

The index r is preferably 0 or 1. It may also be 2.

The index n is preferably 0 or 1, especially 0. It may also be 2.

The index m is preferably 0, 1 or 2, especially 0, or also 1.

Preferably, J is heteroaryl containing at least one, but not more than three N.

Lower alkyl is especially C₁₋₄alkyl, e.g., n-butyl, sec-butyl, tert-butyl, n-propyl, isopropyl or, especially, methyl or also ethyl, or, in the case of Y as lower alkyl, it may be especially isopentyl. Lower alkyl is unsubstituted or substituted by hydroxy or halogen e.g. Br, Cl or F preferably F.

Aryl is preferably an aromatic radical having from 6-14 carbon atoms, especially phenyl, naphthyl, fluorenyl or phenanthrenyl, the mentioned radicals being unsubstituted or substituted by one or more substituents, preferably up to three, especially one or two substituents, especially selected from amino; mono- or di-substituted amino; halogen; alkyl; substituted alkyl; hydroxyl; etherified or esterified hydroxyl; nitro; cyano; carboxy; esterified carboxy; alkanoyl; carbamoyl; N-mono- or N,N-di-substituted carbamoyl; amidino; guanidine; mercapto; sulfo; phenylthio; phenyl-lower alkylthio; alkylphenylthio; phenylsulfinyl; phenyl-lower alkylsulfinyl; alkylphenylsulfinyl; phenylsulfonyl; phenyl-lower alkanesulfonyl; alkylphenylsulfonyl; lower alkenyl, such as ethenyl and phenyl; lower alkylthio, such as methylthio; lower alkanoyl, such as acetyl; lower alkylmercapto, such as methylmercapto (—S—CH₃); halo-lower alkylmercapto, such as trifluoromethylmercapto (—S—CF₃); lower alkanesulfonyl; halo-lower alkanesulfonyl, such as, especially, trifluoromethanesulfonyl, dihydroxybora (—B(OH)₂) and heterocyclyl; and lower alkylenedioxy, such as methylenedioxy, bonded to adjacent carbon atoms of the ring; aryl is preferably phenyl that is unsubstituted or substituted by one or two identical or different substituents from the group consisting of amino; lower alkanoylamino, especially acetylamino; halogen, especially fluorine, chlorine or bromine; lower alkyl, especially methyl, or also ethyl or propyl; halo-lower alkyl, especially trifluoromethyl; hydroxy; lower alkoxy, especially methoxy, or also ethoxy; phenyl-lower alkoxy, especially benzyloxy; and cyano, or (alternatively or additionally to the preceding group of substituents) C₈₋₁₂alkoxy, especially n-decyloxy; carbamoyl; lower alkylcarbamoyl, such as N-methyl- or N-tert-butyl-carbamoyl; lower alkanoyl, such as acetyl or phenyloxy; halo-lower alkyloxy, such as trifluoromethoxy or 1,1,2,2-tetrafluoroethyloxy; lower alkoxycarbonyl, such as ethoxycarbonyl; lower alkylmercapto, such as methylmercapto; halo-lower alkylmercapto, such as trifluoromethylmercapto; hydroxy-lower alkyl, such as hydroxymethyl or 1-hydroxymethyl; lower alkanesulfonyl, such as methanesulfonyl; halo-lower alkanesulfonyl, such as trifluoromethanesulfonyl, phenylsulfonyl, dihydroxybora (—B(OH)₂), 2-methyl-pyrimidin-4-yl, oxazol-5-yl, 2-methyl-1,3-dioxolan-2-yl, 1H-pyrazol-3-yl or 1-methyl-pyrazol-3-yl; and lower alkylenedioxy, such as methylenedioxy, bonded to two adjacent carbon atoms, more especially by one or two identical or different substituents selected from lower alkyl, especially methyl; halogen, especially chlorine or bromine; and halo-lower alkyl, especially trifluoromethyl. Aryl is preferably also naphthyl.

Heteroaryl is preferably an unsaturated heterocyclic radical in the bonding ring and is preferably mono- or also bi- or tri-cyclic; wherein at least in the ring bonding to the radical of the molecule of formula (I) one or more, preferably from 1-4, especially 1 or 2 carbon atoms of a corresponding aryl radical have been replaced by a hetero atom selected from the group consisting of nitrogen, oxygen and sulfur, the bonding ring having preferably from 4-14, especially from 5-7 ring atoms; wherein heteroaryl is unsubstituted or substituted by one or more, especially from 1-3, identical or different substituents from the group consisting of the substituents mentioned above as substituents of aryl; and is especially a heteroaryl radical selected from the group consisting of imidazolyl, thienyl, furyl, pyranyl, thianthrenyl, isobenzofuranyl, benzofuranyl, chromenyl, 2H-pyrrolyl, pyrrolyl, lower alkyl-substituted imidazolyl, benzimidazolyl, pyrazolyl, thiazolyl, isothiazolyl, oxazolyl, isoxazolyl, pyridyl, pyrazinyl, pyrimidinyl, pyridazinyl, indolizinyl, isoindolyl, 3H-indolyl, indolyl, indazolyl, triazolyl, tetrazolyl, purinyl, 4H-quinolizinyl, isoquinolyl, quinolyl, phthalazinyl, naphthyridinyl, quinoxalyl, quinazolinyl, cinnolinyl, pteridinyl, carbazolyl, phenanthridinyl, acridinyl, perimidinyl, phenanthrolinyl and furazanyl, each of those radicals being bonded via a ring having at least one hetero atom to the radical of the molecule of formula (I); pyridyl is especially preferred. Special preference is given also to indolyl that is substituted by halogen, especially by fluorine, especially 6-fluoroindol-3-yl.

Heteroaryl is especially a 5- or 6-membered aromatic heterocycle having 1 or 2 hetero atoms selected from the group consisting of nitrogen, oxygen and sulfur, which heterocycle may be unsubstituted or substituted, especially by lower alkyl, such as methyl; preference is additionally given to a radical selected from 2-methyl-pyrimidin-4-yl, 1H-pyrazol-3-yl and 1-methyl-pyrazol-3-yl.

Heterocycloalkyl is especially a saturated 5- or 6-membered heterocycle having 1 or 2 hetero atoms selected from the group consisting of nitrogen, oxygen and sulfur, which heterocycle may be unsubstituted or substituted, especially by lower alkyl, such as methyl; preference is given to a radical selected from oxazol-5-yl and 2-methyl-1,3-dioxolan-2-yl.

Mono- or di-substituted amino is especially amino that is substituted by one or two identical or different radicals from lower alkyl, such as methyl; hydroxy-lower alkyl, such as 2-hydroxyethyl; phenyl-lower alkyl; lower alkanoyl, such as acetyl; benzoyl; substituted benzoyl, wherein the phenyl radical is unsubstituted or, especially, is substituted by one or more, preferably one or two, substituents selected from nitro and amino, or also from halogen, amino, N-lower alkylamino, N,N-di-lower alkylamino, hydroxy, cyano, carboxy, lower alkoxycarbonyl, lower alkanoyl and carbamoyl; and phenyl-lower alkoxycarbonyl wherein the phenyl radical is unsubstituted or, especially, is substituted by one or more, preferably one or two, substituents selected from nitro and amino, or also from halogen, amino, N-lower alkylamino, N,N-di-lower alkylamino, hydroxy, cyano, carboxy, lower alkoxycarbonyl, lower alkanoyl and carbamoyl; and is preferably N-lower alkylamino, such as N-methylamino or hydroxy-lower alkylamino, such as 2-hydroxyethylamino; phenyl-lower alkylamino, such as benzylamino, N,N-di-lower alkylamino, N-phenyl-lower alkyl-N-lower alkylamino or N,N-di-lower alkylphenylamino; lower alkanoylamino, such as acetylamino; or a substituent selected from the group consisting of benzoylamino and phenyl-lower alkoxycarbonylamino, wherein in each case the phenyl radical is unsubstituted or, especially, is substituted by nitro or amino, or also by halogen, amino, N-lower alkylamino, N,N-di-lower alkylamino, hydroxy, cyano, carboxy, lower alkoxycarbonyl, lower alkanoyl or by carbamoyl, or alternatively or additionally to the preceding group of radicals, by aminocarbonylamino.

Halogen is especially fluorine, chlorine, bromine or iodine, more especially fluorine, chlorine or bromine, in particular fluorine and chlorine.

Alkyl has preferably up to a maximum of 12 carbon atoms and is especially lower alkyl, more especially methyl, or also ethyl, n-propyl, isopropyl or tert-butyl.

Substituted alkyl is alkyl as last defined, especially lower alkyl, preferably methyl, which may contain one or more, especially up to 3 substituents, selected especially from the group consisting of halogen, especially fluorine, and also amino, N-lower alkylamino, N,N-di-lower alkylamino, N-lower alkanoylamino, hydroxy, alkoxy, cyano, carboxy, lower alkoxycarbonyl and phenyl-lower alkoxycarbonyl. Trifluoromethyl is an important substituted alkyl.

Etherified hydroxy is especially C₈₋₂₀alkyloxy, such as n-decyloxy; lower alkoxy (preferred), such as methoxy, ethoxy, isopropyloxy or n-pentyloxy; phenyl-lower alkoxy, such as benzyloxy or also phenyloxy; or, alternatively or additionally to the preceding group, C₈₋₂₀alkyloxy, such as n-decyloxy; halo-lower alkoxy, such as trifluoromethyloxy or 1,1,2,2-tetrafluoroethoxy.

Esterified hydroxy is especially lower alkanoyloxy, benzoyloxy, lower alkoxycarbonyloxy, such as tert-butoxycarbonyloxy; or phenyl-lower alkoxycarbonyloxy, such as benzyloxycarbonyloxy.

Esterified carboxy is especially lower alkoxycarbonyl, such as tert-butoxycarbonyl or ethoxycarbonyl, phenyl-lower alkoxycarbonyl or phenyloxycarbonyl.

Alkanoyl is especially alkyl-carbonyl, more especially lower alkanoyl, e.g., acetyl.

N-Mono- or N,N-di-substituted carbamoyl is especially substituted at the terminal nitrogen by one or two substituents lower alkyl, phenyl-lower alkyl or hydroxy-lower alkyl.

Alkylphenylthio is especially lower alkylphenylthio.

Alkylphenylsulfinyl is especially lower alkylphenylsulfinyl.

Alkylphenylsulfonyl is especially lower alkylphenylsulfonyl.

Pyridyl Y is preferably 3- or 4-pyridyl.

Unsubstituted or substituted cycloalkyl is preferably C₃₋₈cycloalkyl, which is unsubstituted or is substituted in the same manner as aryl, especially as defined for phenyl. Preference is given to cyclohexyl, or also cyclopentyl or cyclopropyl. Preference is given also to 4-lower alkyl-cyclohexyl, such as 4-tert-butylcyclohexyl.

If present, Z is preferably amino; hydroxy-lower alkylamino, such as 2-hydroxyethylamino; lower alkanoylamino, such as acetylamino; nitrobenzoylamino, such as 3-nitrobenzoylamino; aminobenzoylamino, such as 4-aminobenzoylamino; phenyl-lower alkoxycarbonylamino, such as benzyloxycarbonylamino; or halogen, such as bromine; preferably only one substituent is present (m=1), especially one of the last-mentioned substituents, especially halogen. Very special preference is given to a compound of formula (I), or an N-oxide thereof, wherein Z is not present (m=0).

Aryl in the form of phenyl that is substituted by lower alkylenedioxy, such as methylenedioxy, bonded to two adjacent carbon atoms is preferably 3,4-methylenedioxyphenyl.

An N-oxide of a compound of formula (I) is preferably an N-oxide in which an isoquinoline ring nitrogen or a nitrogen in the J moiety carries an oxygen atom, or more than one of the mentioned nitrogen atoms carry an oxygen atom.

Salts are especially the pharmaceutically acceptable salts of compounds of formula (I), or an N-oxide thereof.

Such salts are formed, e.g., by compounds of formula (I), or an N-oxide thereof, having a basic nitrogen atom as acid addition salts, preferably with organic or inorganic acids, especially the pharmaceutically acceptable salts. Suitable inorganic acids are, e.g., hydrohalic acids, such as hydrochloric acid (HCl); sulfuric acid; or phosphoric acid. Suitable organic acids are, e.g., carboxylic phosphonic, sulfonic or sulfamic acids, e.g., acetic acid; propionic acid; octanoic acid; decanoic acid; dodecanoic acid; glycolic acid; lactic acid; 2-hydroxybutyric acid; gluconic acid; glucosemonocarboxylic acid; fumaric acid; succinic acid; adipic acid; pimelic acid; suberic acid; azelaic acid; malic acid; tartaric acid; citric acid; glucaric acid; galactaric acid; amino acids, such as glutamic acid, aspartic acid, N-methylglycine, acetylaminoacetic acid, N-acetylasparagine, N-acetylcysteine, pyruvic acid, acetoacetic acid, phosphoserine, 2- or 3-glycerophosphoric acid, maleic acid, hydroxymaleic acid, methylmaleic acid, cyclohexanecarboxylic acid, benzoic acid, salicylic acid, 1- or 3-hydroxynaphthyl-2-carboxylic acid, 3,4,5-trimethoxybenzoic acid, 2-phenoxybenzoic acid, 2-acetoxybenzoic acid, 4-aminosalicylic acid, phthalic acid, phenylacetic acid, glucuronic acid, galacturonic acid, methane- or ethane-sulfonic acid, 2-hydroxyethanesulfonic acid, ethane-1,2-disulfonic acid, benzenesulfonic acid, 2-naphthalenesulfonic acid, 1,5-naphthalenedisulfonic acid, N-cyclohexylsulfamic acid or N-methyl-, N-ethyl- or N-propyl-sulfamic acid; or other organic protonic acids, such as ascorbic acid.

When negatively charged radicals, such as carboxy or sulfo, are present, salts with bases can also be formed, e.g., metal or ammonium salts, such as alkali metal; alkaline earth metal salts, e.g., sodium, potassium, magnesium or calcium salts; ammonium salts with ammonia or suitable organic amines, such as tertiary monoamines, e.g., triethylamine or tri(2-hydroxyethyl)amine; or heterocyclic bases, e.g., N-ethylpiperidine or N,N′-dimethyl-piperazine.

When a basic group and an acid group are present in the same molecule, a compound of formula (I), or an N-oxide thereof, can also form internal salts.

For isolation or purification it is also possible to use pharmaceutically unacceptable salts, e.g., picrates or perchlorates. Only the pharmaceutically acceptable salts or the free compounds, optionally in the form of pharmaceutical compositions, are used therapeutically, and those are therefore preferred.

In view of the close relationship between the novel compounds in free form and in the form of their salts, including also those salts which can be used as intermediates, e.g., in the purification of the novel compounds or for their identification, hereinbefore and hereinafter any reference to the free compounds is also to be understood as including the corresponding salts, as appropriate and expedient.

In an important embodiment of this invention, J is aryl, preferably heteroaryl as defined above. Thus, an important embodiment of the present invention relates to isoquinoline compounds of formula (Ia) 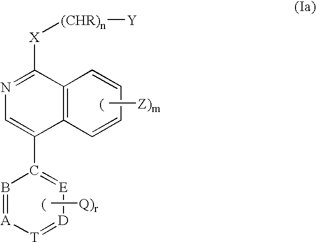
wherein the variable substituents and preferences are the same as described above for the compounds of formula (I).

Preferably, the ring members A, B, D and E are each CH or CQ and the ring member T is N.

Q is bonded to a carbon, preferably bonded to A or to D (r=1) or to both (r=2), so that A and/or D in the case where Q is bonded are C(-Q).

An interesting embodiment of this invention are the compounds of formula (Ia), wherein the ring members A, B, E and T are each CH or CQ and D is N, or wherein the ring members A, B, D and T are each CH or CQ and E is N, or especially wherein the ring members B, D. E and T are each CH or CQ and A is N.

Another especially interesting embodiment of this invention are the compounds of formula (Ia), wherein the ring members A, B and D are each CH or CQ, and E and T are each N or wherein the ring members B. E and T are each CH or CQ and A and D are each N, or wherein the ring members A, D, and T are each CH or CQ and B and E are each N.

And yet another especially interesting embodiment of this invention are compounds of the formula (Ia), wherein the ring members A and D are each CH or CQ and B, T and E are each N.

And another especially interesting embodiment of this invention are compounds, wherein J is a bicyclic heteroaromatic ring system, such as indolyl, isoindolinyl, quinolyl, isoquinolyl, quinazolyl, purinyl, cinnolinyl, naphthyridinyl, phthalazinyl, isobenzofuranyl naphthyridinyl, phthalazinyl, chromenyl and purinyl. A bicyclic heteroaromatic ring system may include Q as a substituent on either ring or on both rings of the bicyclic ring system, and on one or two carbon atoms on either or both rings of the bicyclic ring system.

The inventive compounds inhibit RAF kinase and as such are useful for treating conditions and diseases characterized by an aberrant MAP kinase signaling pathway. Thus the present invention further relates to a method of treating a condition or disease characterized by an aberrant MAP kinase signaling pathway, which comprises administering to a patient an effective RAF kinase inhibiting amount of a compound of formula (I) 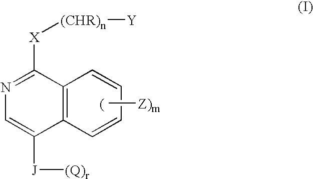
wherein,
n is from 0-2;
r is from 0-2;
m is from 0-4;
J is unsubstituted or substituted once or twice by Q, wherein
J is aryl, heteroaryl, cycloalkyl or heterocycloalkyl, wherein
   aryl is an aromatic radical having from 6-14 carbon atoms, such as phenyl, naphthyl, fluorenyl and phenanthrenyl;
   heteroaryl is an aromatic radical having from 4-14, especially from 5-7 ring atoms, of which 1, 2 or 3 atoms are chosen independently from N, S and O, such as furyl, pyranyl, pyridyl, 1,2-, 1,3- and 1,4-pyrimidinyl, pyrazinyl, triazinyl, triazolyl, oxazolyl, quinazolyl, imidazolyl, pyrrolyl, isoxazolyl isothiazolyl, indolyl, isoindolinyl, quinolyl, isoquinolyl, purinyl, cinnolinyl, naphthyridinyl, phthalazinyl, isobenzofuranyl, chromenyl, purinyl, thianthrenyl, xanthenyl, acridinyl, carbazolyl and phenazinyl;
   cycloalkyl is a cyclic radical having from 3-8, preferably 5-6 ring atoms, such as cyclohexyl and cyclopentyl;
   heterocycloalkyl is a cyclic radical having from 3-8, preferably 5-6 ring atoms, of which 1, 2 or 3 atoms are chosen independently from N, S and O, such as piperidyl, piperazinyl, imidazolidinyl, pyrrolidinyl and pyrazolidinyl;
Q is a substituent on one or two carbon atoms selected from the group consisting of halogen, unsubstituted or substituted lower alkyl, —OR₂, —SR₂, —N(R)R, —NRS(O)₂N(R)R, —NRS(O)₂R, —S(O)R₂, —S(O)₂R₂, —OCOR₂; —C(O)R₂, —CO₂R₂, —NR—COR₂, —CON(R₂)R₂, —S(O)₂N(R₂)R₂, cyano, tri-methylsilanyl, unsubstituted or substituted aryl, unsubstituted or substituted heteroaryl, unsubstituted or substituted cyclyloalkyl, unsubstituted or substituted heterocycloalkyl, —C₁₋₄alkyl-aryl, —C₁₋₄alkyl-heteroaryl, —C₁₋₄alkyl-heterocycloalkyl, —C₁₋₄alkyl-cycloalkyl amino, mono- or di-substituted amino;
R is H or lower alkyl, lower alkoxy;
R₂ is unsubstituted or substituted alkyl, unsubstituted or substituted cycloalkyl, unsubstituted or substituted phenyl, —C₁₋₄alkyl-aryl, —C₁₋₄alkyl-heteroaryl or —C₁₋₄alkyl-heterocycloalkyl;
X is a Y, —N(R)—, oxa, thio; sulfone, sulfoxide, sulfonamide, amide, or ureylene, preferably —NH—; and
Y is H, lower alkyl, substituted or unsubstituted aryl, substituted or unsubstituted heteroaryl or substituted or unsubstituted cycloalkyl;
Z is amino, mono- or di-substituted amino, halogen, alkyl, substituted alkyl, hydroxy, etherified or esterified hydroxy, nitro, cyano, carboxy, esterified carboxy, alkanoyl, carbamoyl, N-mono- or N,N-di-substituted carbamoyl, amidino, guanidino, mercapto, sulfo, phenylthio, phenyl-lower alkylthio, alkylphenylthio, phenylsulfinyl, phenyl-lower alkylsulfinyl, alkylphenylsulfinyl, phenylsulfonyl, phenyl-lower alkanesulfonyl or alkylphenylsulfonyl, and where, if more than one radical Z is present (m≧2), the substituents Z are identical or different;
or an N-oxide of the mentioned compound, wherein one or more N atoms carry an oxygen atom;
   or a pharmaceutically acceptable salt thereof.

The patient is a mammal, generally a human, suffering from a disease that is characterized by an aberrant MAP kinase signaling pathway where aberrant is intended to mean that the signaling through the MAP kinase pathway is excessive relative to normal cells. This can be measured by activation state specific antibodies to pathway members by methods, such as Western blot analysis or immunohistochemistry.

In general, the disease characterized by an aberrant MAP kinase signaling pathway is a proliferative disease, particularly a cancer that expresses mutant B-RAF kinase or which overexpresses wild-type B- or C-RAF kinase. Cancers wherein mutated B-RAF has been detected include melanoma, colorectal cancer, ovarian cancer, prostate, renal, gliomas, adenocarcinomas, sarcomas, breast cancer and liver cancer, preferably melanoma, colorectal cancer, ovarian cancer, gliomas, adenocarcinomas, sarcomas, breast cancer and liver cancer. Mutations of B-RAF kinase are especially prevalent in melanomas.

In accordance with the present invention, a sample of diseased tissue is taken from the patient, for example, as a result of a biopsy or resection, and tested to determine whether the tissue produces mutant B-RAF kinase or overproduces wild-type B- or C-RAF kinase. If the test indicates that mutant B-RAF is produced or that wild-type B- or C-RAF kinase is overproduced in the diseased tissue, the patient is treated by administration of an effective RAF-inhibiting amount of an isoquinoline compound described herein. However, it is also possible to down-regulate the MAP kinase signaling pathway with a RAF kinase inhibiting compound if another kinase in the cascade is the cause of the aberration in the pathway.

Tissue samples are tested by methods generally known in the art. For example, B-RAF mutations are detected by allele specific PCR, DHPLC, mass spectroscopy and over-expression of wild-type B- or C-RAF detected by immunohistochemistry, immunofluoresense or Western blot analysis. A particularly useful method of detecting B-RAF mutations is the polymerase chain reaction based method described in Example A. Similar methods are used to determine whether other kinases in the cascade are mutant or over-expressed.

A particularly important aspect of this invention relates to a method of treating melanoma, which comprises:
(a) testing melanoma tissue from a patient to determine whether the melanoma tissue expresses mutant B-RAF; and
(b) treating the patient with an effective RAF kinase inhibiting amount of an isoquinoline compound described herein if the melanoma tissue is found to express mutant B-RAF.

Generally, the B-RAF mutation is one of those described in the Davies et al. article cited above and listed in Table 1.

TABLE 1

| B-RAF Mutation | Protein Change |
|---|---|
| G1388A | G463E |
| G1388T | G463V |
| G1394C | G465A |
| G1394A | G465E |
| G1394T | G465V |
| G1403C | G468A |
| G1403A | G468E |
| G1753A | E585K |
| T1782G | F594L |
| G1783C | G595R |
| C1786G | L596V |
| T1787G | L596R |
| T1796A | V599E |
| TG1796-97AT | V599D |

Thus, the present invention particularly relates to a method of treating a disease characterized by mutant B-RAF kinase, which comprises detecting a mutation in the B-RAF kinase gene or protein in a tissue sample from a patient and treating the patient with an effective B-RAF kinase inhibiting compound, especially an isoquinoline compound described herein.

A important aspect of this invention includes those instances wherein the mutant B-RAF kinase exhibits a mutation described in Table 1, especially the V599E mutation.

A particularly important aspect of this invention includes those instances wherein disease is melanoma and the mutant B-RAF kinase exhibits a mutation described in Table, 1, especially the V599E mutation.

The RAF kinase inhibiting compounds utilized according to the inventive method include the compounds of formula (I), or N-oxides thereof, which have valuable pharmacological properties, as described above.

In another aspect the invention provide the use of a compound of formula I as pharmaceutical.

In a further aspect of the invention the invention provides the use of a compound of formula I for the preparation of a medicament for the treatment of a disease characterized by an aberrant MAP kinase signaling pathway is a proliferative disease, particularly a cancer that expresses mutant B-RAF kinase or which overexpresses wild-type B- or C-RAF kinase.

A compound of formula (I), or an N-oxide thereof, can be administered on its own or in combination with one or more other therapeutic agents, it being possible for fixed combinations to be used or for a compound according to the invention and one or more other therapeutic agents to be administered in a staggered manner over time or independently of one another, or the combined administration of fixed combinations and of one or more other therapeutic agents is possible. In particular, the administration of a compound of formula (I), or an N-oxide thereof, for tumor treatment can be carried out, alongside or additionally, in combination with chemotherapy (combination with one or more other chemotherapeutic agents, especially cytostatics, or with hormones or compounds having a hormone-like activity), radiotherapy, immunotherapy, surgical treatment or combinations thereof. Long-term therapy is also possible, as is adjuvant therapy in conjunction with other treatment methods, such as those just mentioned. Treatment to maintain the status of a patient after tumor remission or even chemo-preventive treatment, e.g., in the case of at risk patients, is also possible.

There come into consideration as therapeutic agents with which the compounds according to the invention can be combined especially one or more anti-proliferative, cytostatic or cytotoxic compounds, e.g., one or more chemotherapeutic agents selected from the group comprising an inhibitor of polyamine biosynthesis; an inhibitor of a different protein kinase, especially protein kinase C or of a tyrosine protein kinase, such as epidermal growth factor receptor protein tyrosine kinase; an inhibitor of a growth factor, such as vascular endothelial growth factor; a cytokine; a negative growth regulator, such as TGF-β or IFN-β, an aromatase inhibitor; hormones or hormone analogues; and a conventional cytostatic agent.

Compounds according to the invention are intended not only for the (prophylactic and, preferably, therapeutic) treatment of human beings, but also for the treatment of other warm-blooded animals, e.g., of commercially-useful animals, e.g., rodents, such as mice, rabbits or rats; or guinea pigs.

In general, the invention relates also to the use of a compound of formula (I), or an N-oxide thereof, in inhibiting RAF kinase activity.

A compound of formula (I), or an N-oxide thereof, can also be used for diagnostic purposes, e.g., in order that tumors obtained from warm-blooded animals, especially human beings, as the original “host” and transplanted into mice, can be examined for reduced growth after addition of such a compound, in order thus to study their sensitivity to the compound in question, thus allowing possible methods of treatment for a tumor disease in the original host to be ascertained and determined better.

In the groups of preferred compounds of formula (I) mentioned below, definitions of substituents from the above-mentioned general definitions may expediently be used, e.g., in order to replace more general definitions by definitions that are more specific or, especially, by definitions that are indicated as being preferred; preference is in each case given to the definitions indicated above as being preferred or mentioned by way of example.

Preference is given to a compound of formula (Ia) 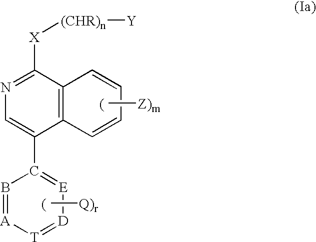
wherein
n is from 0-2;
r is from 0-2;
m is from 0-4;
A, B, D, E and T are each CH or CQ or
A, B, D and E are each CH or CQ and T is N or
B, D, E and T are each CH or CQ and A is N or
A, B, T and E are each CH or CQ and D is N or
A, B, D, and T are each CH or CQ and E is N or
A, B and D are each CH or CQ and E and T are N or
B, E, and T are each CH or CQ and A and D are each N or
A, D and T are each CH or CQ and B and E are each N or
A and D are each CH or CQ and B, E and T are each N;
Q is a substituent on one or two carbon atoms selected from the group consisting of halogen, unsubstituted or substituted lower alkyl, —OR₂, —SR₂, —NR₂, —NRS(O)₂N(R)₂, —NRS(O)₂R, —S(O)R₂, —S(O)_{z}R₂, —OCOR₂, —C(O)R₂, —CO₂R₂, —NR—COR₂, —CON(R₂)₂, —S(O)₂N(R₂)₂, cyano, tri-methylsilanyl, unsubstituted or substituted aryl, unsubstituted or substituted heteroaryl, unsubstituted or substituted cycloalkyl, unsubstituted or substituted heterocycloalkyl, —C₁₋₄alkyl-aryl, —C₁₋₄alkyl-heteroaryl, —C₁₋₄alkyl-heterocyclyl, amino, mono- or di-substituted amino;
R is H or lower alkyl;
R₂ is unsubstituted or substituted alkyl, unsubstituted or substituted cycloalkyl, phenyl, —C₁₋₄alkyl-aryl, —C₁₋₄alkyl-heteroaryl or —C₁₋₄alkyl-heterocycloalkyl;
X is Y, —N(R)—, oxa, thio, sulfone, sulfoxide, sulfonamide, amide or ureylene;
Y is H, lower alkyl, substituted or unsubstituted aryl, substituted or unsubstituted heteroaryl, substituted or unsubstituted cycloalkyl or substituted or unsubstituted heterocycloalkyl; and
Z is amino, mono- or di-substituted amino, halogen, alkyl, substituted alkyl, hydroxy, etherified or esterified hydroxy, nitro, cyano, carboxy, esterified carboxy, alkanoyl, carbamoyl, N-mono- or N,N-di-substituted carbamoyl, amidino, guanidino, mercapto, sulfo, phenylthio, phenyl-lower alkylthio, alkylphenylthio, phenylsulfinyl, phenyl-lower alkylsulfinyl, alkylphenylsulfinyl, phenylsulfonyl, phenyl-lower alkanesulfonyl or alkylphenylsulfonyl, and where, if more than one radical Z is present (m≧2), the substituents Z are identical or different;
   or an N-oxide or a pharmaceutically acceptable salt thereof.

Preference is also given to a compound of formula (Ia),

wherein

r is from 0-2;
n is 0 or 1;
m is 0 or 1;
A, B, D and E are each CH or CQ and T is N or
A, B, T and E are each CH or CQ and D is N or
A, B and D are each CH or CQ and E and T are each N;
Q is a substituent on one or two carbon atoms selected from the group consisting of halogen, unsubstituted or substituted lower alkyl, —OR₂, —SR₂, —NR₂, —NRS(O)₂N(R)₂, —NRS(O)₂R, —S(O)R₂, —S(O)₂R₂, —OCOR₂, —C(O)R₂, —CO₂R₂, —NR—COR₂, —CON(R₂)₂, —S(O)₂N(R₂)₂, cyano, tri-methylsilanyl, unsubstituted or substituted aryl, unsubstituted or substituted heteroaryl, unsubstituted or substituted cycloalkyl, unsubstituted or substituted heterocycloalkyl, —C₁₋₄alkyl-aryl, —C₁₋₄alkyl-heteroaryl, —C₁₋₄alkyl-heterocyclyl, amino, mono- or di-substituted amino;
R is H or lower alkyl;
R₂ is unsubstituted or substituted alkyl, unsubstituted or substituted cycloalkyl, phenyl, —C₁₋₄alkyl-aryl, —C₁₋₄alkyl-heteroaryl or —C₁₋₄alkyl-heterocycloalkyl;
X is —NR—, oxa or thia;
Y is phenyl that is unsubstituted or substituted by one or two identical or different substituents selected from the group consisting of amino; lower alkanoylamino, halogen, lower alkyl, halo-lower alkyl, hydroxy; lower alkoxy, phenyl-lower alkoxy, and cyano, or alternatively or additionally to the preceding group of substituents, lower alkenyl, C₈₋₁₂alkoxy, lower alkoxycarbonyl, carbamoyl, lower alkylcarbamoyl, lower alkanoyl, halo-lower alkyloxy, lower alkoxycarbonyl, lower alkylmercapto, halo-lower alkylmercapto, hydroxy-lower alkyl, lower alkanesulfonyl, halo-lower alkanesulfonyl, phenylsulfonyl, dihydroxybora (—B(OH)₂) and lower alkylenedioxy or
Y is pyridyl; and
Z is halogen; amino; N-lower alkylamino; hydroxy-lower alkylamino; phenyl-lower alkylamino; N,N-di-lower alkylamino; N-phenyl-lower alkyl-N-lower alkylamino; N,N-di-lower alkylphenylamino; lower alkanoylamino, such as acetylamino; or a substituent selected from the group consisting of benzoylamino and phenyl-lower alkoxycarbonylamino, wherein the phenyl radical in each case is unsubstituted or is substituted by nitro or by amino, or also by halogen, amino, N-lower alkylamino, N,N-di-lower alkylamino, hydroxy, cyano, carboxy, lower alkoxycarbonyl, lower alkanoyl or by carbamoyl;
   or an N-oxide or a pharmaceutically acceptable salt thereof.

Special preference is also given to a compound of formula (Ia),

wherein

r is from 0-2, preferably 1;

n is 0 or 1;

m is 1 or, especially, 0;

A, B, D and E are each CH or CQ and T is N or

A, B, T and E are each CH or CQ and D is N or

A, B and D are each CH or CQ and E and T are each N;
Q is preferably bonded to A, to D or to A and D; and is selected from halogen, especially fluorine, chlorine or bromine; lower alkyl, especially methyl, or also, ethyl or propyl; hydroxy; lower alkoxy, especially methoxy, or also, ethoxy; 2-hydroxyethoxy; 2-methoxyethoxy; (2-(1H-imidazol-1-yl)ethoxy, or also, hydroxyiminomethyl; lower alkanoyl, such as acetyl or formyl; lower alkylmercapto, such as methylmercapto or amino; N-lower alkylamino, such N-methylamino, or also N-ethylamino, N-(n)-propyl- or N-isopropylamino; 2-cyanoethylamino; 3-(methoxyphenyl)amino; 3-(4-morpholinyl)propylamino; 3-(pyridinyl)methylamino; 2-(2-pyridinyl)ethylamino; 4-(1H-imidazol-1-yl)butylamino; 4-(trifluoromethoxyphenyl)amino); (methylaminosulfonyl)amino; (methylsulfonyl)amino; (tetrahydro-2H-pyran-4-yl)amino; (tetrahydro-2H-pyran-4-yl)methylamino; (tetrahydro-3-furanyl)amino; (2-(1H-imidazol-1-yl)ethyl)amino, or also, hydroxy-lower alkylamino, such as 2-hydroxyethylamino or (2-methoxyethyl)methylamino; 2-(2-hydroxyethoxy)ethylamino; spirans, including 1,4-dioxa-8-azaspiro[4.5]dec-8-yl; substituted or unsubstituted heterocyclyl, such as 1-azetidinyl, 3-ethoxycarbonyl-1-azetidinyl or 3-carboxy-1-azetidinyl; or also, tetrahydro-2H-1,3-oxazinyl; dihydro-1,2,5-oxathiazin-5(6H)-yl; tetrahydro-1(2H)-pyrimidinyl); 3-(acetyltetrahydro)-1(2H)-pyrimidinyl; piperazinyl; 4-(2-hydroxyethyl)-1-piperazinyl; 4-(ethoxycarbonyl)-1-piperazinyl; 4-acetyl-1-piperazinyl; or especially piperidinyl, 4-(trifluormethyl)-1-piperidinyl, 4-(difluoromethyl)-1-piperidinyl, 4-(phenylmethyl)-1-piperidinyl, 4-phenoxy-1-piperidinyl, 4-cyano-1-piperidinyl, 4-methoxy-1-piperidinyl, 4-ethoxycarbonyl-1-piperidinyl, 4-hydroxy-1-piperidinyl, 4-carboxy-1-piperidinyl, 4-(aminocarbonyl)-1-piperidinyl, 4-methylthio-1-piperidinyl, 4-methylsulfonyl-1-piperidinyl, (tetrahydro-2H-pyran-4-yl)oxy, or also, especially, 4-morpholinyl, 3,5-dimethylmorpholinyl or 2-phenyl-4-morpholinyl;
   R is H or lower alkyl, especially H or methyl;
   X is —NR—, oxa or thia, especially —NH—;
   Y is phenyl that is unsubstituted or substituted by one or two identical or different substituents selected from the group consisting of amino; lower alkanoylamino, especially acetylamino; halogen, especially fluorine, chlorine or bromine; lower alkyl, especially tert-butyl, or also methyl, ethyl or propyl; halo-lower alkyl, especially trifluoromethyl; hydroxy; lower alkoxy, especially methoxy, or also ethoxy; phenyl-lower alkoxy, especially benzyloxy; and cyano, or (alternatively or additionally to the preceding group of substituents) lower alkenyl, such as ethenyl, C₈₋₁₂alkoxy, especially n-decyloxy; lower alkoxycarbonyl, such as tert-butoxycarbonyl; carbamoyl; lower alkylcarbamoyl, such as N-methyl- or N-tert-butyl-carbamoyl; lower alkanoyl, such as acetyl; phenyloxy; halo-lower alkyloxy, such as trifluoromethoxy or 1,1,2,2-tetrafluoroethyloxy; lower alkoxycarbonyl, such as ethoxycarbonyl; lower alkylmercapto, such as methylmercapto; halo-lower alkylmercapto, such as trifluoromethylmercapto; hydroxy-lower alkyl, such as hydroxymethyl or 1-hydroxymethyl; lower alkanesulfonyl, such as methanesulfonyl; halo-lower alkanesulfonyl, such as trifluoromethanesulfonyl; phenylsulfonyl; dihydroxybora (—B(OH)₂); 2-methyl-pyrimidin-4-yl; oxazol-5-yl; 2-methyl-1,3-dioxolan-2-yl; 1H-pyrazol-3-yl; 1-methyl-pyrazol-3-yl; and lower alkylenedioxy, such as methylenedioxy, bonded to two adjacent carbon atoms, especially by one or two substituents selected from halogen, such as chlorine or bromine; lower alkyl, such as methyl; and halo-lower alkyl, such as trifluoromethyl or
   Y is pyridyl, especially 3-pyridyl or
   Y is especially phenyl; 2-, 3- or 4-aminophenyl; 2-, 3- or 4-acetylaminophenyl; 2-, 3- or 4-fluorophenyl; 2-, 3- or 4-chlorophenyl; 2-, 3- or 4-bromophenyl; 2,3-, 2,4-, 2,5 or 3,4-dichlorophenyl; chloro-fluoro-phenyl, such as 3-chloro-4-fluoro-phenyl; or also 4-chloro-2-fluoroanilino; 2-, 3- or 4-methylphenyl; 2-, 3- or 4-ethylphenyl; 2-, 3- or 4-propylphenyl; methyl-fluoro-phenyl, such as 3-fluoro-4-methylphenyl; 2-, 3- or 4-trifluoromethylphenyl; 2-, 3- or 4-hydroxyphenyl; 2-, 3- or 4-methoxyphenyl; 2-, 3- or 4-ethoxyphenyl; methoxy-chloro-phenyl, such as 3-chloro-4-methoxycarbonyl; 2-, 3- or 4-benzyloxyphenyl; 2-, 3- or 4-cyanophenyl; or also 2-, 3- or 4-pyridyl or
   Y is more especially 4-chlorophenyl; 2-, 3- or 4-methylphenyl; 4-chloro-5-trifluoromethylphenyl; 3-bromo-5-trifluoromethylphenyl or
   Y is very especially 3,5-dimethylphenyl; or also is especially 4-methyl-3-iodophenyl, 3,4-bis(trifluoromethyl)phenyl, 3-bromo-4-ethyl-phenyl or 3-chlorobenzylphenyl;
Z is amino; N-lower alkylamino, such as N-methylamino; hydroxy-lower alkylamino, such as 2-hydroxyethylamino; phenyl-lower alkylamino, such as benzylamino; N,N-di-lower alkylamino; N-phenyl-lower alkyl-N-lower alkylamino; N,N-di-lower alkylphenylamino; lower alkanoylamino, such as acetylamino; or a substituent selected from the group consisting of benzoylamino and phenyl-lower alkoxycarbonylamino, wherein the phenyl radical in each case is unsubstituted or, especially, is substituted by nitro or by amino, or also by halogen, amino, N-lower alkylamino, N,N-di-lower alkylamino, hydroxy, cyano, carboxy, lower alkoxycarbonyl, lower alkanoyl or by carbamoyl or
Z is halogen, especially bromine; more especially amino, acetylamino, nitrobenzoylamino, aminobenzoylamino, 2-hydroxyethylamino, benzyloxycarbonylamino or bromine; and
   or an N-oxide or a pharmaceutically acceptable salt thereof.

Special preference is given to a compound of formula (Ia),

wherein

r is 1;
n is 0;
m is 0;
B, D, E, and T are CH or CQ and A is N (3-pyridyl), or especially
A, B, D and E are each CH or CQ and T is N (4-pyridyl);
Q is a substituent on preferably one, or also two carbon atoms selected from halogen, especially fluorine or chlorine; lower alkyl, especially methyl; or also ethyl or propyl; amino, N-lower alkylamino, such as N-methylamino; or also N-ethylamino, N-(n)-propyl- or N-isopropylamino; or 2-cyanoethylamino, 3-(methoxyphenyl)amino or 3-(4-morpholinyl)propylamino, 3-(pyridinyl)methylamino, 2-(2-pyridinyl)ethylamino, 4-(1H-imidazol-1-yl)butylamino, 4-(trifluoromethoxyphenyl)amino), (methylaminosulfonyl)amino, (methylsulfonyl)amino, (tetrahydro-2H-pyran-4-yl)amino, (tetrahydro-2H-pyran-4-yl)methylamino, (tetrahydro-3-furanyl)amino, (2-(1H-imidazol-1-yl)ethyl)amino; or also hydroxy-lower alkylamino, such as 2-hydroxyethylamino, 2-(2-hydroxyethoxy)ethylamino, substituted or unsubstituted heterocyclyl, especially tetrahydro-1(2H)-pyrimidinyl); or 3-(acetyltetrahydro)-1(2H-pyrimidinyl; or also piperazinyl, 4-(2-hydroxyethyl)-1-piperazinyl, 4-(ethoxycarbonyl)-1-piperazinyl, 4-acetyl-1-piperazinyl; or especially piperidinyl, 4-(trifluormethyl)-1-piperidinyl, 4-(difluoromethyl)-1-piperidinyl, 4-(phenylmethyl)-1-piperidinyl, 4-phenoxy-1-piperidinyl, 4-cyano-1-piperidinyl, 4-methoxy-1-piperidinyl, 4-ethoxycarbonyl-1-piperidinyl, 4-hydroxy-1-piperidinyl, 4-carboxy-1-piperidinyl, 4-(aminocarbonyl)-1-piperidinyl, 4-methylthio-1-piperidinyl, 4-methylsulfonyl-1-piperidinyl; or also especially 4-morpholinyl, 3,5-dimethylmorpholinyl or 2-phenyl-4-morpholinyl;
R is H or lower alkyl, especially H or methyl;
X is —NR—, especially —NH—;
Y is phenyl that is unsubstituted or substituted by one or two identical or different substituents selected from the group consisting of halogen, especially fluorine, or more especially, chlorine or bromine; lower alkyl, especially methyl; isopropyl and tert-butyl; and halo-lower alkyl, especially trifluoromethyl, 4-chlorophenyl, 2-, 3- or 4-methylphenyl, 4-chloro-5-trifluoromethylphenyl, 3-bromo-5-trifluoromethylphenyl, or more especially 3,5-dimethylphenyl; or also, 4-methyl-3-iodophenyl, 3,4-bis(trifluoromethyl)phenyl or 3-bromo-4-ethyl-phenyl;
   or an N-oxide or pharmaceutically acceptable salt thereof.

Special preference is given also to a compound of formula (Ia),

wherein

r is 1;
n is from 0-2;
m is 0;
A, B, D and E are each CH or CQ and T is N;
Q is a substituent on one carbon atom selected from amino, N-lower alkylamino, such as N-methylamino; or also N-ethylamino, N-(n)-propyl- or N-isopropylamino; or 2-cyanoethylamino, 3-(methoxyphenyl)amino, 3-(4-morpholinyl)propylamino, 3-(pyridinyl)methylamino, 2-(2-pyridinyl)ethylamino, 4-(1H-imidazol-1-yl)butylamino, 4-(trifluoromethoxyphenyl)amino), (methylaminosulfonyl)amino, (methylsulfonyl)amino, (tetrahydro-2H-pyran-4-yl)amino, (tetrahydro-2H-pyran-4-yl)methylamino, (tetrahydro-3-furanyl)amino, (2-(1H-imidazol-1-yl)ethyl)amino; or also hydroxy-lower alkylamino, such as 2-hydroxyethylamino, 2-(2-hydroxyethoxy)ethylamino, substituted or unsubstituted heterocyclyl, especially piperidinyl, 4-(trifluormethyl)-1-piperidinyl, 4-(difluoromethyl)-1-piperidinyl, 4-(phenylmethyl)-1-piperidinyl, 4-phenoxy-1-piperidinyl, 4-cyano-1-piperidinyl, 4-methoxy-1-piperidinyl, 4-ethoxycarbonyl-1-piperidinyl, 4-hydroxy-1-piperidinyl, 4-carboxy-1-piperidinyl, 4-(aminocarbonyl)-1-piperidinyl, 4-methylthio-1-piperidinyl, 4-methylsulfonyl-1-piperidinyl; or also most preferably morpholinyl;
R is H;
X is —NR—, especially —NH—; and
Y is phenyl that is unsubstituted or substituted by halogen, especially chlorine, or by lower alkyl, such as methyl or trifluoromethyl or isopropyl; or especially tert-butyl; lower alkoxy, especially methoxy, such as 4-chlorophenyl, 4-methoxyphenyl or 4-trifluoromethoxyphenyl; naphthyl; cyclohexyl that is unsubstituted or substituted by lower alkyl, especially by tert-butyl, such as 4-tert-butyl-cyclohexyl; indolyl that is unsubstituted or substituted by halogen, especially by fluorine, especially 6-fluoroindol-3-yl; or lower alkyl, especially isopentyl;
   or an N-oxide or pharmaceutically acceptable salt thereof.

In particular, preference is given also to a compound of formula (Ia),

wherein

r is 1;
n is 0;
m is 0;
A, B, D and E are each CH and T is N;
R is H;
X is —NH—,
Y is phenyl that is substituted by one or two identical or different substituents selected from halogen and lower alkyl. Special preference is given to compounds, wherein Y is phenyl that is substituted in the 4-position by tert-butyl or trifluoromethyl; and
Q is a substituent on one carbon atom selected from morpholinyl;
or an N-oxide or pharmaceutically acceptable salt thereof.

Another interesting embodiment of the invention is a compound of formula (I) 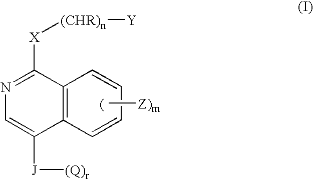
wherein
n is from 0-2;
r is from 0-2;
m is from 0-4;
J is a bicyclic heteroaromatic ring system, such as indolyl, isoindolinyl, quinolyl, isoquinolyl, quinazolyl, purinyl, cinnolinyl, naphthyridinyl, phthalazinyl, isobenzofuranyl naphthyridinyl, phthalazinyl, chromenyl and purinyl;
Q is a substituent on either one or both rings of the bicyclic ring system, and on one or two carbon atoms on either one or both rings of the bicyclic ring system, selected from the group consisting of halogen, unsubstituted or substituted lower alkyl, —OR₂, —SR₂, —NR₂, —NRS(O)₂N(R)₂, —NRS(O)₂R, —S(O)R₂, —S(O)₂R₂, —OCOR₂, —C(O)R₂, —CO₂R₂, —NR—COR₂, —CON(R₂)₂, —S(O)₂N(R₂)₂, cyano, tri-methylsilanyl, unsubstituted or substituted aryl, unsubstituted or substituted heteroaryl, unsubstituted or substituted cycloalkyl, unsubstituted or substituted heterocycloalkyl, —C₁₋₄alkyl-aryl, —C₁₋₄alkyl-heteroaryl, —C₁₋₄alkyl-heterocyclyl, amino, mono- or di-substituted amino;
R is H or lower alkyl;
R₂ is unsubstituted or substituted alkyl, unsubstituted or substituted cycloalkyl, phenyl, —C₁₋₄alkyl-aryl, —C₁₋₄-alkyl-heteroaryl or —C₁₋₄alkyl-heterocycloalkyl;
X is Y, —N(R)—, oxa, thio, sulfone, sulfoxide, sulfonamide, amide or ureylene;
Y is H, lower alkyl, substituted or unsubstituted aryl, substituted or unsubstituted heteroaryl, substituted or unsubstituted cycloalkyl or substituted or unsubstituted heterocycloalkyl; and
Z is amino, mono- or di-substituted amino, halogen, alkyl, substituted alkyl, hydroxy, etherified or esterified hydroxy, nitro, cyano, carboxy, esterified carboxy, alkanoyl, carbamoyl, N-mono- or N,N-di-substituted carbamoyl, amidino, guanidino, mercapto, sulfo, phenylthio, phenyl-lower alkylthio, alkylphenylthio, phenylsulfinyl, phenyl-lower alkylsulfinyl, alkylphenylsulfinyl, phenylsulfonyl, phenyl-lower alkanesulfonyl or alkylphenylsulfonyl, and where, if more than one radical Z is present (m≧2), the substituents Z are identical or different;
   or an N-oxide or a pharmaceutically acceptable salt thereof.

And yet another interesting embodiment of the invention is a compound of formula (I),

wherein

n is 0;
r is 0;
m is 0;
J is a bicyclic heteroaromatic ring system, such as indolyl, isoindolinyl, quinolyl, isoquinolyl, quinazolyl, purinyl, cinnolinyl, naphthyridinyl, phthalazinyl, isobenzofuranyl naphthyridinyl, phthalazinyl, chromenyl and purinyl;
R is H or lower alkyl;
X is Y, —N(R)—, oxa, thio, sulfone, sulfoxide, sulfonamide, amide or ureylene; and
Y is H, lower alkyl, substituted or unsubstituted aryl, substituted or unsubstituted heteroaryl, substituted or unsubstituted cycloalkyl or substituted or unsubstituted heterocycloalkyl;
or an N-oxide or a pharmaceutically acceptable salt thereof.

And yet another interesting embodiment of the invention is a compound of formula (I),

wherein

n is 0;
r is 0;
m is 0;
J is isoquinolyl;
X is NH; and
Y is, substituted or unsubstituted aryl, especially tert-butylphenyl, very especially 4-tert-butylphenyl;
   or an N-oxide or a pharmaceutically acceptable salt thereof.

The compounds according to the invention can be prepared by processes known per se for other compounds, especially by:

a) reacting a compound of formula (II)

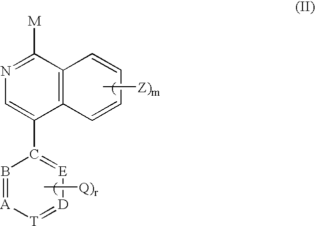

wherein
r, m, A, B, D, E, T, Q and Z are as defined for a compound of formula (Ia); and
M is a nucleofugal leaving group, with a compound of formula (III) 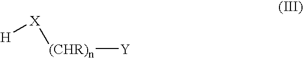
wherein n, R, X and Y are as defined for a compound of formula (I), functional groups in the compounds of formula (II) and of formula (III) that are not to take part in the reaction being in protected form, if necessary, and removing any protecting groups that are present, wherein the starting compounds mentioned in process a) may also be in the form of salts where a salt-forming group is present and reaction in the salt form is possible;
and, if desired, converting a resulting compound of formula (I), or an N-oxide thereof, into a different compound of formula (I), or an N-oxide thereof, converting a free compound of formula (I), or an N-oxide thereof, into a salt, converting a resulting salt of a compound of formula (I), or of an N-oxide thereof, into the free compound or into a different salt, and/or separating a mixture of isomeric compounds of formula (I), or its N-oxide, into the individual isomers.

### DETAILED DESCRIPTION OF THE PROCESS VARIANTS

In the following, more detailed description of the preparation process, r, n, m, A, B, D, E, T, Q, R, X, Y and Z and are as defined for compounds of formula (Ia), unless indicated otherwise.

Process a)

In the compound of formula (II), a nucleofugal leaving group M is especially halogen, more especially bromine, iodine or, very especially, chlorine.

The reaction between the compound of formula II and the compound of formula (III) takes place in suitable inert polar solvents, especially alcohols, e.g., lower alkanols, such as methanol, propanol or, especially, ethanol or n-butanol; or it takes place in a melt without the addition of a solvent, especially when one of the reactants is in liquid form. The reaction takes place at elevated temperatures, preferably from approximately 60° C. to reflux temperature, e.g., under reflux conditions or at a temperature of from approximately 90° C. to approximately 110° C. The compound of formula (III) can also be used in the form of a salt, e.g., in the form of an acid addition salt with a strong acid, such as a hydrogen halide, e.g., in the form of the hydrochloride salt; or the corresponding acid, e.g., HCl, can be added in a suitable solvent, e.g., an ether, such as dioxane.

Alternatively, the reaction between the compound of formula (II) and the compound of formula (III) takes place in suitable, inert polar solvents, especially ethers, e.g., tetrahydrofuran (THF); or in a melt without the addition of a solvent, especially if one of the reaction partners is present in liquid form. The reaction takes place at elevated temperatures, preferably between about 80° C. and 140° C. in a pressure tube. The compound of formula (III) can be used as a salt, e.g., as an basic addition salt with a strong base, such as potassium hydroxide or sodium hydride.

Where one or more other functional groups, e.g., carboxy, hydroxy, amino or mercapto, in a compound of formula (II) and/or (III) are present in protected form or must be present in protected form because they are not to take part in the reaction, the protecting groups are groups which are customarily used in the synthesis of peptide compounds, but also in the synthesis of cephalosporins and penicillins, as well as of nucleic acid derivatives and sugars. The protecting groups may already be present in the precursors and are to protect the functional groups in question against undesired secondary reactions, such as acylations, etherifications, esterifications, oxidations, solvolysis and the like. The protecting groups for functional groups in starting materials whose reaction is to be avoided, especially carboxy, amino, hydroxy and mercapto groups, include especially those protecting groups (conventional protecting groups) which are customarily used in the synthesis of peptide compounds, cephalosporins, penicillins or nucleic acid derivatives and sugars. The protecting groups may already be present in the precursors and are to protect the functional groups in question against undesired secondary reactions, such as acylations, etherifications, esterifications, oxidations, solvolysis, etc. In some cases the protecting groups can cause the reactions to proceed selectively, e.g., stereoselectively. It is a characteristic of protecting groups that they can be removed easily, that is to say without undesired secondary reactions, e.g., by solvolysis, by reduction, by photolysis or enzymatically, e.g., also under conditions analogous to physiological conditions, and that they are not present in the end products. The person skilled in the art will know or can readily find out which protecting groups are suitable in the reactions mentioned hereinbefore and hereinafter.

The protection of functional groups by means of such protecting groups, the protecting groups themselves, and reactions for their removal are described, e.g., in standard works, such as Protective Groups in Organic Chemistry, McOmie, Ed., Plenum Press, London and NY (1973); Protective Groups in Organic Synthesis, 3^{rd} edition, Greene, Ed., Wiley, NY (1999); The Peptides; Volume 3, Gross and Meienhofer, Eds., Academic Press, London and NY (1981); Methoden der organischen Chemie, Houben Weyl, 4^{th} edition, Volume 15/l, Georg Thieme Verlag, Ed., Stuttgart (1974); Aminosäuren, Peptide, Proteine, Jakubke and Jescheit, Eds., Verlag Chemie, Weinheim, Deerfield Beach and Basle (1982); and Chemie der Kohlenhydrate: Monosaccharide und Derivate, Jochen Lehmann, Ed., Georg Thieme Verlag, Stuttgart (1974).

Protecting groups mentioned in the Examples are preferably introduced and, if required, removed analogously to the mentioned methods.

Additional Process Steps

In the additional process steps, which are carried out if desired, functional groups in the starting compounds that are not to take part in the reaction may be present in unprotected form or in protected form, e.g., protected by one or more of the protecting groups mentioned above under process a). All or some of the protecting groups are then removed by one of the methods mentioned under process a).

Salts of compounds of formula (I), or an N-oxide thereof, having a salt-forming group can be prepared in a manner known per se. For example, acid addition salts of compounds of formula (I) or their N-oxides can be obtained, e.g., by treatment with an acid or a suitable anion exchange reagent. It is also possible to convert salts having two acid molecules, e.g., a dihalide of a compound of formula (I), or of an N-oxide thereof, into salts having one acid molecule per compound of formula (I), or N-oxide thereof, e.g., into a monohalide; that can be achieved, e.g., by heating to the molten state or, e.g., by heating in solid form under a high vacuum at elevated temperature, e.g., from 130-170° C., one molecule of the acid being expelled per molecule of a compound of formula (I), or of an N-oxide thereof.

Salts can be converted into the free compounds in customary manner, e.g., by treatment with a suitable basic agent, e.g., with alkali metal carbonates; hydrogen carbonates or hydroxides, e.g., potassium carbonate or sodium hydroxide.

Stereoisomeric mixtures, e.g., mixtures of diastereoisomers, can be separated into the corresponding isomers in a manner known per se by means of suitable separating procedures. For example, diastereoisomeric mixtures can be separated into the individual diastereoisomers by fractional crystallization, chromatography, solvent partitioning and the like. The separation may be carried out either at the stage of one of the starting materials or in the case of the compounds of formula (I) themselves. Enantiomers can be separated by formation of diastereoisomeric salts, e.g., by salt formation with an enantiomerically pure chiral acid, or by chromatographic methods, e.g., by chromatography, e.g., HPLC, on chromatographic carrier materials with chiral ligands.

A compound of formula (I) can be converted into a corresponding N-oxide. The reaction is carried out with a suitable oxidizing agent, preferably a peroxide, e.g., m-chloroperbenzoic acid, in a suitable solvent, e.g., a halogenated hydrocarbon, such as chloroform or methylene chloride; or in a lower alkanecarboxylic acid, such as acetic acid, preferably at a temperature of from 0° C. to the boiling temperature of the reaction mixture, especially approximately room temperature.

A compound of formula (I), or an N-oxide thereof, wherein Z is lower alkanoylamino can be hydrolyzed to the corresponding amino compound (Z=amino), e.g., by hydrolysis with an inorganic acid, especially HCl, in aqueous solution, it being possible to add further solvents, preferably at elevated temperature, e.g., under reflux.

A compound of formula (I), or an N-oxide thereof, wherein Z is amino substituted by one or two identical or different radicals selected from lower alkyl, hydroxy-lower alkyl and phenyl-lower alkyl can be converted into the compound that is correspondingly substituted at the amino group, e.g., by reaction with a lower alkyl halide, a hydroxy-lower alkyl halide, which is hydroxy-protected if necessary (see process a); or a phenyl-lower alkyl halide under reaction conditions analogous to those mentioned under process a). For the introduction of 2-hydroxy-lower alkyl substituents at the amino group Z, addition starting from an epoxide, e.g., ethylene oxide, is also possible. The addition is carried out especially in aqueous solution and/or in the presence of polar solvents, such as alcohols, e.g., methanol, ethanol, isopropanol or ethylene glycol; ethers, such as dioxane; amides, such as dimethyl formamide; or phenols, such as phenol; also under anhydrous conditions, in apolar solvents, such as benzene and toluene; or in benzene/water emulsions, optionally in the presence of acid or basic catalysts, e.g., of alkaline solutions, such as sodium hydroxide solution; or in the presence of hydrazine-doped solid phase catalysts, such as aluminium oxide; in ethers, e.g., diethyl ether, generally at temperatures of approximately from 0° C. to the boiling temperature of the reaction mixture in question, preferably at from 20° C. to reflux temperature, where appropriate under elevated pressure, e.g., in a bomb tube, whereby the boiling temperature may also be exceeded, and/or under an inert gas, such as nitrogen or argon. Reductive alkylation of an amino group Z with a lower alkane aldehyde, a phenyl-lower alkane aldehyde or a hydroxy-lower alkane aldehyde, which is hydroxy-protected if necessary, is also possible. The reductive alkylation preferably takes place with hydrogenation in the presence of a catalyst, especially a noble metal catalyst, such as platinum or, especially, palladium, which is preferably bonded to a support material, such as carbon; or a heavy metal catalyst, such as Raney nickel, at normal pressure or at pressures of from 0.1-10 megapascals (MPa); or with reduction by means of complex hydrides, such as boron hydrides, especially alkali metal cyanoborohydrides, e.g., sodium cyanoborohydride, in the presence of a suitable acid, preferably of a relatively weak acid, such as a lower alkanecarboxylic acid or, especially, a sulfonic acid, such as p-toluenesulfonic acid; in customary solvents, e.g., alcohols, such as methanol or ethanol; or ethers, e.g., cyclic ethers, such as THF, in the absence or presence of water.

In a compound of formula (I), or an N-oxide thereof, an amino group Z can be converted by acylation into an amino group that is substituted by lower alkanoyl, benzoyl, substituted benzoyl or by phenyl-lower alkoxycarbonyl wherein the phenyl radical is unsubstituted or substituted. The corresponding acids contain a free carboxy group or are in the form of reactive acid derivatives thereof, e.g., in the form of the derived activated esters or reactive anhydrides, also reactive cyclic amides. The reactive acid derivatives can also be formed in situ. Activated esters are especially esters that are unsaturated at the linking carbon atom of the radical to be esterified, e.g., of the vinyl ester type, such as vinyl esters, obtainable, e.g., by transesterification of a corresponding ester by vinyl acetate or activated vinyl ester method; carbamoyl esters obtainable, e.g., by treating the corresponding acid with an isoxazolium reagent, 1,2-oxazolium, or Woodward method; or 1-lower alkoxyvinyl esters obtainable, e.g., by treating the corresponding acid with a lower alkoxyacetylene, or ethoxyacetylene method; or esters of the amidino type, such as N,N-disubstituted amidino esters obtainable, e.g., by treating the corresponding acid with a suitable N,N′-disubstituted carbodiimide, e.g., N,N′-dicyclohexylcarbodiimide or, especially, N-(3-dimethylaminopropyl)-N′-ethylcarbodiimide, or carbodiimide method; or N,N-disubstituted amidino esters obtainable, e.g., by treating the corresponding acid with an N,N-disubstituted cyanamide, or cyanamide method; suitable aryl esters, especially phenyl esters suitably substituted by electrophilic substituents obtainable, e.g., by treating the corresponding acid with a suitably substituted phenol, e.g., 4-nitrophenol, 4-methylsulfonylphenol, 2,4,5-trichlorophenol, 2,3,4,5,6-pentachlorophenol or 4-phenyldiazophenol, in the presence of a condensing agent, such as N,N′-dicyclohexylcarbodiimide, or activated aryl esters method; cyanomethyl esters obtainable, e.g., by treating the corresponding acid with chloroacetonitrile in the presence of a base, or cyanomethyl esters method; thioesters, especially unsubstituted or substituted, e.g., nitro-substituted, phenylthio esters, obtainable, e.g., by treating the corresponding acid with unsubstituted or substituted, e.g., nitrosubstituted, thiophenols, inter alia by means of the anhydride or carbodiimide method, or activated thiolesters method; or, especially, amino or amido esters obtainable, e.g., by treating the corresponding acid with an N-hydroxyamino or N-hydroxyamido compound, e.g., N-hydroxysuccinimide, N-hydroxypiperidine, N-hydroxyphthalimide, N-hydroxy-5-norbornene-2,3-dicarboxylic acid imide, 1-hydroxybenztriazole or 3-hydroxy-3,4-dihydro-1,2,3-benztriazin-4-one, e.g., by the anhydride or carbodiimide method, or activated N-hydroxy esters method. Internal esters, e.g., γ-lactones, can also be used. Anhydrides of acids may be symmetrical or, preferably, mixed anhydrides of those acids, e.g., anhydrides with inorganic acids, such as acid halides, especially acid chlorides obtainable, e.g., by treating the corresponding acid with thionyl chloride, phosphorus pentachloride, phosgene or oxalyl chloride, or acid chloride method; azides obtainable, e.g., from a corresponding acid ester via the corresponding hydrazide and treatment thereof with nitrous acid, or azide method; anhydrides with carbonic acid semiesters, e.g., carbonic acid lower alkyl semiesters, especially chloroformic acid methyl esters obtainable, e.g., by treating the corresponding acid with chloroformic acid lower alkyl esters or with a 1-lower alkoxycarbonyl-2-lower alkoxy-1,2-dihydroquinoline, or mixed O-alkylcarbonic acid anhydrides method; or anhydrides with dihalogenated, especially dichlorinated, phosphoric acid obtainable, e.g., by treating the corresponding acid with phosphorus oxychloride or phosphorus oxychloride method; anhydrides with other phosphoric acid derivatives, e.g., those which can be obtained with phenyl N-phenylphosphoramidochloridate, or by reacting alkylphosphoric acid amides in the presence of sulfonic acid anhydrides and/or racemization reducing additives, such as N-hydroxybenzotriazole, or in the presence of cyanophosphonic acid diethyl ester; or with phosphorous acid derivatives, or anhydrides with organic acids, such as mixed anhydrides with organic carboxylic acids obtainable, e.g., by treating the corresponding acid with an unsubstituted or substituted lower alkane- or phenyl-lower alkane-carboxylic acid halide, e.g., phenylacetic acid, pivalic acid or trifluoroacetic acid chloride, or mixed carboxylic acid anhydrides method; or with organic sulfonic acids obtainable, e.g., by treating a salt, such as an alkali metal salt, of the corresponding acid with a suitable organic sulfonic acid halide, such as lower alkane- or aryl-, e.g., methane- or p-toluene-sulfonic acid chloride, or mixed sulfonic acid anhydrides method; as well as symmetrical anhydrides obtainable, e.g., by condensing the corresponding acid in the presence of a carbodiimide or of 1-diethylaminopropyne or symmetrical anhydrides method. Suitable cyclic amides are especially amides with 5-membered diazacycles of aromatic nature, such as amides with imidazoles, e.g., imidazole obtainable, e.g., by treating the corresponding acid with N,N′-carbonyldiimidazole, or imidazole method; or pyrazole, e.g., 3,5-dimethylpyrazole obtainable, e.g., via the acid hydrazide by treatment with acetylacetone, or pyrazolide method. As mentioned, derivatives of carboxylic acids, which are used as acylating agents, can also be formed in situ. For example, N,N′-disubstituted amidino esters can be formed in situ by reacting the mixture of the starting material of formula (I) and the acid used as acylating agent in the presence of a suitable N,N′-disubstituted carbodiimide, e.g., N,N′-dicyclohexylcarbodiimide or, especially, N-(3-dimethylaminopropyl)-N′-ethylcarbodiimide. Furthermore, amino or amido esters of the acids used as acylating agent can be formed in the presence of the starting material of formula (I) to be acylated, by reacting a mixture of the corresponding acid and amino starting materials in the presence of an N,N′-disubstituted carbodiimide, e.g., N,N′-dicyclohexylcarbodiimide, and of an N-hydroxyamine or N-hydroxyamide, e.g., N-hydroxysuccinimide, optionally in the presence of a suitable base, e.g., 4-dimethylaminopyridine. Moreover, activation can be achieved in situ by reaction with N,N,N′,N′-tetraalkyluronium compounds, such as O-benztriazol-1-yl-N,N,N′,N′-tetramethyluronium hexafluorophosphate, O-(1,2-dihydro-2-oxo-1-pyridyl)-N,N,N′,N′-tetramethyluronium tetrafluoroborate (in the absence or presence of 1,8-diazabicyclo[5.4.0]undec-7-ene-(1,5,5)) or O-(3,4-dihydro-4-oxo-1,2,3-benztriazolin-3-yl)-N,N,N′,N′-tetramethyluronium tetrafluoroborate. Finally, phosphoric acid anhydrides of the carboxylic acids can be prepared in situ by reacting an alkylphosphoric acid amide, such as hexamethylphosphoric acid triamide, in the presence of a sulfonic acid anhydride, such as 4-toluenesulfonic acid anhydride, with a salt, such as a tetrafluoroborate, e.g., sodium tetrafluoroborate, or with a different derivative of hexamethylphosphoric acid triamide, such as benzotriazol-1-yl-oxy-tris(dimethylamino)phosphonium hexafluoride, preferably in the presence of a racemization-reducing additive, such as N hydroxybenztriazole. If desired, an organic base is added, preferably a tertiary amine, e.g., a tri-lower alkylamine, especially ethyldiisopropylamine or, more especially, triethylamine, and/or a heterocyclic base, e.g., 4-dimethylaminopyridine or, preferably, N-methylmorpholine or pyridine. The condensation is preferably carried out in an inert, aprotic, preferably anhydrous solvent or solvent mixture, e.g., in a carboxylic acid amide, e.g., formamide or dimethylformamide; a halogenated hydrocarbon, e.g., methylene chloride, carbon tetrachloride or chlorobenzene; a ketone, e.g., acetone; a cyclic ether, e.g., THF or dioxane; an ester, e.g., ethyl acetate; or a nitrile, e.g., acetonitrile, or in a mixture thereof, where appropriate at reduced or elevated temperature, e.g., in a temperature range of from approximately −40° C. to approximately +100° C., preferably from approximately −10° C. to approximately +70° C., where arylsulfonyl esters are used also at approximately from +100-200° C., especially at temperatures of from 10-30° C., and, where appropriate, under an inert gas atmosphere, e.g., a nitrogen or argon atmosphere. Aqueous, e.g., alcoholic; solvents, e.g., ethanol; or aromatic solvents, e.g., benzene or toluene, are also possible.

A nitro group Z in a compound of formula (I) can be reduced to an amino group, e.g., by reduction with metals or selective hydrogenation; e.g., by reaction with magnesium/ammonium sulfate in a water/alcohol mixture, such as methanol/water, at elevated temperature, e.g., from 30-60° C. (see Synth Commun, Vol. 25, No. 2, pp. 4025-4028 (1995)); by reaction with zinc/borohydride In an acid amide, such as dimethylformamide, at temperatures below room temperature, e.g., at approximately 0° C.; by reaction with 1,1′-dioctyl-4,4′-bipyridinium dibromide/sodium tetrathionate/potassium carbonate in water/halogenated hydrocarbon mixtures, e.g., water/methylene chloride mixtures, at elevated temperature, e.g., from 25-35° C. (see Tetrahedron Lett, Vol. 34, No. 46, pp. 7445-7446 (1993)); with sodium borohydride on Amberlyte IRA-400 ion exchanger in the chloride form in an alcohol, such as methanol/water, at preferred temperatures of from 0-40° C. (see Synth Commun, Vol. 19, Nos. 5/6, pp. 805-811 (1989)); with potassium borohydride in a halogenated hydrocarbon/alcohol mixture, e.g., methylene chloride/methanol, at preferred temperatures of from 10-35° C. (see Synth Commun, Vol. 19, No. 17, pp. 3047-3050 (1989)); with sodium borohydride in dioxane; with borane in THF; by hydrogenation in the presence of Pd/C in an alcohol at a preferred temperature of from 0-35° C. and in the presence of ammonium formate (see Tetrahedron Lett, Vol. 25, No. 32, pp. 3415-3418 (1989)); with titanium tetrachloride/lithium aluminium hydride or titanium tetrachloride/magnesium in an ether, such as THF (see Bull Chem Soc Belg, Vol. 97, No. 1, pp. 51-53 (1988)); or with ferric ammonium chloride/water at elevated temperature, preferably under reflux. See Synth. Commun, Vol. 22, pp. 3189-3195 (1992).

General Process Conditions

All the process steps mentioned in the present text can be carried out under reaction conditions which are known per se, preferably those mentioned specifically, in the absence or, customarily, in the presence of solvents or diluents, preferably those which are inert towards the reagents used and are solvents therefor, in the absence or presence of catalysts, condensing agents or neutralizing agents, e.g., ion exchangers, such as cation exchangers, e.g., in the H⁺ form, depending on the nature of the reaction and/or of the reactants at reduced, normal or elevated temperature, for example in a temperature range of from approximately −100° C. to approximately 190° C., preferably from approximately −80° C. to approximately 150° C., e.g., at from −80° C. to −60° C., at room temperature, at from −20° C. to 40° C. or at the boiling point of the solvent used, under atmospheric pressure or in a closed vessel, where appropriate under pressure; and/or in an inert atmosphere, e.g., under an argon or nitrogen atmosphere.

In all starting materials and intermediate compounds, salts can be present where salt-forming groups are present. Salts can also be present during the reaction of such compounds, provided that the reaction is not impaired thereby.

At all stages of the reaction, isomeric mixtures that form can be separated into the individual isomers, e.g., diastereoisomers or enantiomers, or into any desired mixtures of isomers, e.g., racemates or diastereoisomeric mixtures, e.g., analogously to the methods described under “Additional process steps”.

In certain cases, e.g., in the case of hydrogenations, it is possible to achieve stereoselective reactions so that, e.g., it is easier to obtain individual isomers.

The solvents from which those that are suitable for a particular reaction can be selected include, e.g., water; esters, such as lower alkyl lower alkanoates, e.g., diethyl acetate; ethers, such as aliphatic ethers, e.g., diethyl ether or cyclic ethers, e.g., THF; liquid aromatic hydrocarbons, such as benzene or toluene; alcohols, such as methanol, ethanol or 1- or 2-propanol; nitriles, such as acetonitrile; halogenated hydrocarbons, such as methylene chloride; acid amides, such as dimethylformamide; basses, such as heterocyclic nitrogen bases, e.g., pyridine; carboxylic acids, such as lower alkanecarboxylic acids, e.g., acetic acid; carboxylic acid anhydrides, such as lower alkanoic acid anhydrides, e.g., acetic anhydride; cyclic, linear or branched hydrocarbons, such as cyclohexane, hexane or isopentane; or mixtures of those solvents, e.g., aqueous solutions, unless indicated otherwise in the description of the processes. Such solvent mixtures can also be used in working up, e.g., by chromatography or partitioning.

The invention relates also to those forms of the process in which a compound obtainable as an intermediate at any stage is used as starting material and the remaining steps are carried out, or the process is interrupted at any stage, or a starting material is formed under the reaction conditions or is used in the form of a reactive derivative or salt, or a compound obtainable by the process according to the invention is produced under the process conditions and is processed further in situ. There are preferably used those starting materials which lead to the compounds described above as being preferred, especially as being especially preferred, more especially preferred and/or very especially preferred.

The preparation of compounds of formula (I), or N-oxides thereof, is preferably carried out analogously to the processes and process steps mentioned in the Examples.

The compounds of formula (I), or N-oxides thereof, including their salts, can also be obtained in the form of hydrates, or their crystals can include, e.g., the solvent used for crystallization (presence in the form of solvates).

Pharmaceutical Compositions, Methods and Uses

The present invention relates also to pharmaceutical compositions which comprise a compound of formula (I), or an N-oxide thereof, as active ingredient and can be used especially in the treatment of the diseases mentioned at the beginning. Special preference is given to compositions for enteral, such as nasal, buccal, rectal or, especially, oral and parenteral, such as intravenous, intramuscular or subcutaneous, administration to warm-blooded animals, especially human beings. The compositions comprise the active ingredient on its own or, preferably, together with a pharmaceutically acceptable carrier. The dose of active ingredient depends on the disease to be treated and on the species, its age, weight and individual condition, individual pharmacokinetic data and on the mode of administration.

The invention relates also to pharmaceutical compositions for use in a method of treating the human or animal body prophylactically or, especially, therapeutically, to a process for their preparation (especially in the form of compositions for the treatment of tumours) and to a method of treating the above-mentioned diseases, especially tumor diseases, more especially those mentioned above.

The invention relates also to processes, and to the use of compounds of formula (I), or an N-oxide thereof, for the preparation of pharmaceutical compositions comprising compounds of formula (I), or an N-oxide thereof, as active component (active ingredient).

Preference is given to a pharmaceutical composition which is suitable for administration to a warm-blooded animal, especially a human being or a commercially useful mammal, which is suffering from a disease characterized by an aberrant MAP kinase signaling pathway especially, a tumor disease, most particularly melanoma, comprising a compound of formula (I), or an N-oxide thereof, or a pharmaceutically acceptable salt thereof where salt-forming groups are present, in an amount that is effective in inhibiting RAF kinase, particularly a mutant RAF kinase, together with at least one pharmaceutically acceptable carrier.

Preference is given also to a pharmaceutical composition for the prophylactic or, especially, therapeutic treatment of tumor diseases and other proliferative diseases in a warm-blooded animal, especially a human being or a commercially useful mammal, which requires such treatment, especially which is suffering from such a disease, comprising a novel compound of formula (I), or an N-oxide thereof, or a pharmaceutically acceptable salt thereof, as active ingredient in an amount that is effective prophylactically or, especially, therapeutically against the mentioned diseases.

Pharmaceutical compositions comprise from approximately 1% to approximately 95% active ingredient, dosage forms that are in single dose form preferably comprising from approximately 20% to approximately 90% active ingredient, and dosage forms that are not in single dose form preferably comprising from approximately 5% to approximately 20% active ingredient. Unit dose forms are, e.g., dragées, tablets, ampoules, vials, suppositories or capsules. Other dosage forms are, e.g., ointments, creams, pastes, foams, tinctures, lipsticks, drops, sprays, dispersions, etc. Examples are capsules comprising from approximately 0.05 g to approximately 1.0 g of the active ingredient.

The pharmaceutical compositions of the present invention are prepared in a manner known per se, e.g., by means of conventional mixing, granulating, confectioning, dissolving or lyophilizing processes.

Solutions of the active ingredient are preferably used, in addition also suspensions or dispersions, especially isotonic aqueous solutions, dispersions or suspensions, which, in the case of, e.g., lyophilized compositions which contain the active substance alone or together with a carrier, e.g., mannitol, can be prepared prior to use. The pharmaceutical compositions may be sterilized and/or comprise excipients, e.g., preservatives, stabilizers, wetting agents and/or emulsifiers, solubilizers, salts for regulating the osmotic pressure and/or buffers, and are prepared in a manner known per se, e.g., by means of conventional dissolving or lyophilizing processes. The mentioned solutions or suspensions may comprise viscosity-increasing substances, such as sodium carboxymethylcellulose, carboxymethylcellulose, dextran, polyvinylpyrrolidone or gelatin, or solubilizers, e.g., Tween 80 [polyoxyethylene(20)sorbitan monooleate; trademark of ICI Americas, Inc., USA].

Suspensions in oil comprise as the oily component the vegetable, synthetic or semi-synthetic oils customary for injection purposes. There may be mentioned as such, especially liquid fatty acid esters, which comprise as the acid component a long-chained fatty acid having from 8-22 carbon atoms, especially from 12-22 carbon atoms, e.g., lauric acid, tridecylic acid, myristic acid, pentadecylic acid, palmitic acid, margaric acid, stearic acid, arachidic acid, behenic acid or corresponding unsaturated acids, e.g., oleic acid, elaidic acid, erucic acid, brassidic acid or linoleic acid, optionally with the addition of antioxidants, e.g., vitamin E, β-carotene or 3,5-di-tert-butyl-4-hydroxytoluene. The alcohol component of those fatty acid esters has a maximum of 6 carbon atoms and is a mono- or poly-hydric, e.g., mono-, di- or tri-hydric, alcohol, e.g., methanol, ethanol, propanol, butanol or pentanol or their isomers, but especially glycol and glycerol. Examples of fatty acid esters which may be mentioned are, therefore ethyl oleate, isopropyl myristate, isopropyl palmitate, “Labrafil M 2375” (polyoxyethyleneglycerol trioleate from Gattefossé, Paris), “Labrafil M 1944 CS” (unsaturated polyglycolized glycerides prepared by alcoholysis of apricot kernel oil and composed of glycerides and polyethylene glycol ester; Gattefossé, France), “Labrasol” (saturated polyglycolized glycerides prepared by alcoholysis of TCM and composed of glycerides and polyethylene glycol ester; Gattefossé, France) and/or “Miglyol 812” (triglyceride of saturated fatty acids having a chain length of from C₈₋₁₂ from Hüls AG, Germany), but especially vegetable oils, such as cottonseed oil, almond oil, olive oil, castor oil, sesame oil, soybean oil and, more especially, groundnut oil.

The preparation of the injection compositions is carried out in customary manner under sterile conditions, as are also the introduction thereof, e.g., into ampoules or vials and the sealing of the containers.

Pharmaceutical compositions for oral administration can be obtained, e.g., by combining the active ingredient with one or more solid carriers, granulating a resulting mixture, where appropriate, and processing the mixture or granules, if desired, where appropriate by addition of additional excipients, to tablets or dragée cores.

Suitable carriers are especially fillers, such as sugars, e.g., lactose, saccharose, mannitol or sorbitol; cellulose preparations and/or calcium phosphates, e.g., tricalcium phosphate or calcium hydrogen phosphate; also binders, such as starches, e.g., corn, wheat, rice or potato starch, methylcellulose, hydroxypropylmethylcellulose, sodium carboxymethylcellulose and/or polyvinylpyrrolidone; and/or, if desired, disintegrators, such as the above-mentioned starches, also carboxymethyl starch; cross-linked polyvinylpyrrolidone, alginic acid or a salt thereof, such as sodium alginate. Additional excipients are especially flow conditioners and lubricants, e.g., silicic acid, talc, stearic acid or salts thereof, such as magnesium or calcium stearate, and/or polyethylene glycol; or derivatives thereof.

Dragée cores can be provided with suitable, optionally enteric, coatings, there being used inter alia concentrated sugar solutions which may contain gum arabic, talc, polyvinylpyrrolidone, polyethylene glycol and/or titanium dioxide or coating solutions in suitable organic solvents or solvent mixtures or, for the preparation of enteric coatings, solutions of suitable cellulose preparations, such as acetylcellulose phthalate or hydroxypropylmethylcellulose phthalate. Colorings or pigments may be added to the tablets or dragée coatings, e.g., for identification purposes or to indicate different doses of active ingredient.

Pharmaceutical compositions for oral administration are also hard gelatin capsules and soft sealed capsules consisting of gelatin and a plasticizer, such as glycerol or sorbitol. The hard gelatin capsules may contain the active ingredient in the form of granules, e.g., in admixture with fillers, such as corn starch; binders and/or glidants, such as talc or magnesium stearate; and optionally stabilizers. In soft capsules the active ingredient is preferably dissolved or suspended in suitable liquid excipients, such as fatty oils, paraffin oil or liquid polyethylene glycols or fatty acid esters of ethylene glycol or propylene glycol, it likewise being possible to add stabilizers and detergents, e.g., of the polyoxyethylenesorbitan fatty acid ester type.

Suitable rectally administrable pharmaceutical compositions are, e.g., suppositories that consist of a combination of the active ingredient with a suppository base. Suitable suppository bases are, e.g., natural or synthetic triglycerides, paraffin hydrocarbons, polyethylene glycols or higher alkanols.

For parenteral administration there are suitable, especially aqueous solutions of an active ingredient in water-soluble form, e.g., in the form of a water-soluble salt; or aqueous injection suspensions that comprise viscosity-increasing substances, e.g., sodium carboxymethylcellulose, sorbitol and/or dextran; and, if desired, stabilizers. The active ingredient, optionally together with excipients, can also be in the form of a lyophilisate and can be made into a solution prior to parenteral administration by the addition of suitable solvents.

Solutions used, e.g., for parenteral administration can also be used as infusion solutions.

Preferred preservatives are, e.g., antioxidants, such as ascorbic acid; or microbicides, such as sorbic acid or benzoic acid.

The invention relates especially to a process or a method for treating one of the pathological conditions that is characterized by an aberrant MAP kinase signaling pathway, especially a disease responsive to inhibition of RAF kinase, especially a corresponding tumour disease. The compounds of formula (I), or an N-oxide thereof, can be administered prophylactically or therapeutically as such or in the form of pharmaceutical compositions, preferably in an amount that is effective against the mentioned diseases, to a warm-blooded animal, e.g., a human being, requiring such treatment, the compounds being used especially in the form of pharmaceutical compositions. In the case of a body weight of approximately 70 kg, a daily dose of from approximately 0.1 g to approximately 5 g, preferably from approximately 0.5 g to approximately 2 g, of a compound of the present invention is administered.

The preferred dosage, composition and preparation of pharmaceutical formulations (medicaments) to be used in each particular case are described above.

Starting Materials

The starting materials used and the reaction conditions chosen are preferably such that the compounds mentioned as being preferred are obtained.

The starting materials of formulae (II) and (III) are known, can be prepared by processes known per se, or are available commercially; in particular, they can be prepared by processes analogous to those mentioned in the Examples.

In the preparation of starting materials, any functional groups present that are not to take part in the reaction may be in protected form, if necessary. Preferred protecting groups, their introduction and their removal are described under process a) or in the Examples. Instead of the starting materials and intermediates in question, it is also possible to react salts thereof where salt-forming groups are present and the reaction in question is also possible using a salt. Therefore, any reference hereinbefore and hereinafter to starting materials is also intended to include their salts, where expedient and possible.

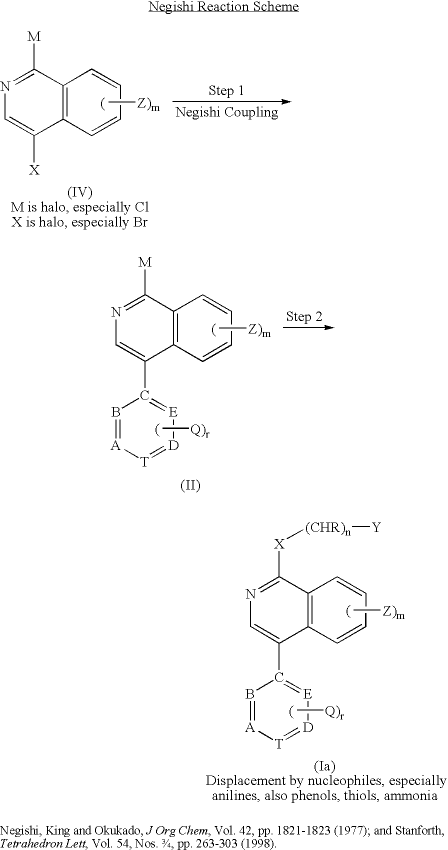

As to the individual steps in the above scheme, Step 1 involves reacting a compound of formula (IV) in a palladium mediated cross-coupling reaction of two suitable coupling partners, preferably under Negishi conditions. The palladium-mediated coupling of a compound of formula (IV) is conducted in the presence of:
1) an organo-metallic reagent, preferably an organolithium reagent such as n-butyllithium;
2) a zinc halide such as zinc bromide;
3) a palladium reagent such as tetrakis(triphenylphosphine)-palladium(0);
4) a suitable coupling partner, such as the bromide, chloride, iodide or triflate of J-Q defined in Table 2; and
5) an organic solvent, preferably an ether, more preferably a cyclic ether, such as THF, at a temperature between −78° C. and 25° C., preferably at −78° C. for a period between 10 minutes and 48 hours.

Step 2 involves the reaction of a compound of formula (II) with a compound of formula (III) 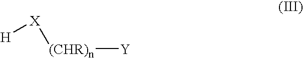
wherein
n, R, X and Y are as defined for a compound of formula (I), functional groups in the compounds of formulae (II) and (III) that are not to take part in the reaction being in protected form, if necessary, and removing any protecting groups that are present, wherein the starting compounds mentioned in process a) may also be in the form of salts where a salt-forming group is present and reaction in the salt form is possible;
and, if desired, converting a resulting compound of formula (Ia), or an N-oxide thereof, into a different compound of formula (Ia), or an N-oxide thereof, converting a free compound of formula (Ia), or an N-oxide thereof, into a salt, converting a resulting salt of a compound of formula (Ia), or of an N-oxide thereof, into the free compound or into a different salt, and/or separating a mixture of isomeric compounds of formula (Ia), or its N-oxide, into the individual isomers.

The reaction between the compound of formula (II) and the compound of formula (III) takes place in suitable inert polar solvents, especially alcohols, e.g., lower alkanols, such as methanol, propanol or, especially, ethanol or n-butanol, or it takes place in a melt without the addition of a solvent, especially when one of the reactants is in liquid form. The reaction takes place at elevated temperatures, preferably from approximately 60° C. to reflux temperature, e.g., under reflux conditions or at a temperature of from approximately 60-110° C. The compound of formula (III) can also be used in the form of a salt, e.g., in the form of an acid addition salt with a strong acid, such as a hydrogen halide, e.g., in the form of the hydrochloride salt; or the corresponding acid, e.g., HCl, can be added in a suitable solvent, e.g., an ether, such as dioxane.

Alternatively, the reaction between the compound of formula (II) and the compound of formula (III) takes place in suitable, inert polar solvents, especially ethers, e.g., THF, or in a melt without the addition of a solvent, especially if one of the reaction partners is present in liquid form. The reaction-takes place at elevated temperatures, preferably between about 80° C. and 140° C. in a pressure tube. The compound of formula (III) can be used as a salt, e.g., as an basic addition salt with a strong base, such as potassium hydroxide or sodium hydride.

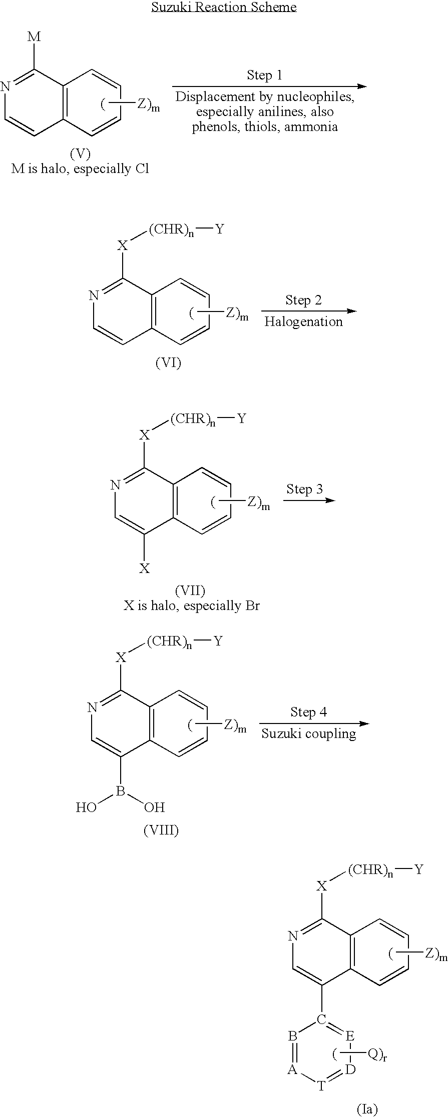

Step 1 involves the reaction of a compound of formula (V) with a compound of formula (III) 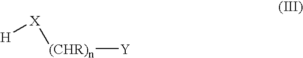
wherein n, R, X and Y are as defined for a compound of formula (I), functional groups in the compounds of formulae (V) and (III) that are not to take part in the reaction being in protected form, if necessary, and removing any protecting groups that are present, wherein the starting compounds mentioned in process a) may also be in the form of salts where a salt-forming group is present and reaction in the salt form is possible.

The reaction between the compound of formula (V) and the compound of formula (III) takes place in suitable inert polar solvents, especially alcohols, e.g., lower alkanols, such as methanol, propanol or, especially, ethanol or n-butanol, or it takes place in a melt without the addition of a solvent, especially when one of the reactants is in liquid form. The reaction takes place at elevated temperatures, preferably from approximately 60° C. to reflux temperature, e.g., under reflux conditions or at a temperature of from approximately 60-110° C. The compound of formula (III) can also be used in the form of a salt, e.g., in the form of an acid addition salt with a strong acid, such as a hydrogen halide, e.g., in the form of the hydrochloride salt; or the corresponding acid, e.g., HCl, can be added in a suitable solvent, e.g., an ether, such as dioxane.

Alternatively, the reaction between the compound of formula (V) and the compound of formula (III) takes place in suitable, inert polar solvents, especially ethers, e.g., THF, or in a melt without the addition of a solvent, especially if one of the reaction partners is present in liquid form. The reaction takes place at elevated temperatures, preferably between about 80° C. and 140° C. in a pressure tube. The compound of formula (III) can be used as a salt, e.g., as an basic addition salt with a strong base, such as potassium hydroxide or sodium hydride.

Step 2 involves the halogenation, especially bromination of the isoquinolyl nucleus of a compound of formula (VI) in the presence of an electrophilic halogenating agent, preferably phenyltrimethylammonium tribromide in an inert polar solvent, preferably THF at a temperature between 0° C. and the reflux temperature of the solvent, preferably at room temperature for a period of time between 1 hour and 24 hours, preferably for 12 hours to provide a compound of formula (VII).

Step 3 Involves the preparation of a boronic acid intermediate. The reaction is conducted in the presence of:
1) an organo-metallic reagent, preferably an organolithium reagent such as n-butyllithium;
2) a source of electrophilic boron, such as Bis(pinocolato)diboron or such as a trialkylborate, such as triisopropyl borate; and
3) a polar organic solvent, preferably an ether, more preferably a cyclic ether, such as THF, at a temperature between −78° C. and 25° C., preferably at −78° C. for a period between 10 minutes and 48 hours, preferably for 4.5 hours to provide a compound of formula (VIII).

Step 4 involves the palladium mediated cross-coupling reaction of two suitable coupling partners, preferably under Suzuki conditions. The palladium-mediated coupling is conducted in the presence of:
1) a suitable Suzuki cross-coupling partner, such as the bromide, chloride, iodide or triflate of J-Q defined in Table 2;
2) a palladium reagent such as tetrakis(triphenylphosphine)-palladium(0) or dichlorobis(triphenylphosphine)-palladium(II);
3) a base, such as potassium carbonate; and
4) a polar organic solvent, such as an ether or dimethyl formamide, preferably at 60° C. for a period between 10 minutes and 48 hours to provide a compound of formula (Ia), which may be a final product or an intermediate compound.

A compound of formula (Ia) can act as an intermediate compound if A, B, E, D or T have a leaving group. In that case, an amine, oxygen or sulfur nucleophile acts to displace the leaving group, resulting in an alternative final compound of formula (Ia). This synthesis involves the reaction between the compound of formula (Ia), wherein Q comprises a reactive group; and a compound of formula (Q-H), where Q is selected from OR₂, —SR₂, —NR₂, —NRS(O)₂N(R)₂, —NRS(O)₂R takes place in suitable, inert polar solvents, especially alcohols, e.g., lower alkanols, such as methanol, propanol or, especially ethanol or n-butanol, or in a melt without the addition of a solvent, especially if one of the reaction partners is present in liquid form. The reaction takes place at elevated temperatures, preferably between about 60° C. and the reflux temperature, e.g., under reflux conditions, or at a temperature between approximately 90° C. and approximately 110° C. The compound of formula (Q) can be used as a salt, e.g., as an acid addition salt with a strong acid, such as hydrogen halide, e.g., as a hydrochloride salt.

Alternatively, the reaction between the compound of formula (Ia) and the compound of formula (Q-H), as defined above, takes place in suitable, inert polar solvents, especially ethers, e.g., THF, or in a melt without the addition of a solvent, especially if one of the reaction partners is present in liquid form. The reaction takes place at elevated temperatures, preferably between about 80° C. and 140° C. in a pressure tube. The compound of formula (III) can be used as a salt, e.g., as an basic addition salt with a strong base, such as potassium hydroxide or sodium hydride.

The other starting materials are known, can be prepared by processes known per se, or are available commercially or, in particular, can be prepared by processes analogous to those mentioned in the Examples.

The Examples which follow serve to illustrate the invention without limiting the scope thereof.

### EXAMPLE 1

### (4-tert-Butyl-phenyl)-[6-fluoro-4-(2-morpholin-4-yl-pyrimidin-4-yl)-isoquinolin-1-yl]-amine

(4-tert-Butyl-phenyl)-[6-fluoro-4-(4,4,5,5-tetramethyl-[1,3,2]dioxaborolan-2-yl)-isoquinolin-1-yl]-amine is dissolved in diethylether (3 mL) and 2,4-dichloropyrimidine (117 mg, 0.785 mmol) and K₂CO₃ (291 mg, 2.141 mmol) is added and the solution is degassed for 10 minutes. Approximately 10 mg Pd(PPh₃)₄ is added and the mixture is heated at 60° C. overnight with stirring. To the mixture is added water, followed by extraction with diethyl ether. The solution is passed through a silica gel pad and is concentrated by evaporation. To the concentrate is added 1 mL of morpholine, followed by heating at 80° C. overnight. The mixture is rotary evaporated and purified by preparing TLC and then preparing HPLC (35-65% CH₃CN/water in 0.1% TFA). The residue is dissolved in ethyl acetate and washed with saturated NaHCO₃, brine and dried over MgSO₄. The solute is removed to give a brown solid (6 mg). M+H⁺=458.25.

¹H NMR (300 MHz) (DMSO); δ 1.31 (s, 9H), 3.70 (m, 4H), 3.77 (m, 4H), 7.02 (d, 1H, J=5.14 Hz), 7.38 (d, 2H, J=8.44 Hz), 7.59 (m, 1H), 7.72 (d, 2H, J=8.80 Hz), 8.30 (m, 2H), 8.47 (d, 1H, J=5.14 Hz), 8.72 (dd, 1H, J=5.87, 9.17 Hz), 9.51 (s, 1H).

The starting material is prepared as follows:

### 1a) 1-Chloro-6-fluoro-isoquinoline

6-Fluoro-2H-isoquinolin-1-one (1.3 g, 7.97 mmol) (for preparation, see PCT/GB02/00514 and WO 02/062816) is suspended in CH₃CN (20 mL) and then POCl₃ (3.7 g, 23.9 mmol). 4 N HCl (2 mL) in dioxane (2 mL) is added dropwise. The resulting mixture is heated at 50° C. overnight with stirring. The reaction mixture is poured into a saturated NaHCO₃ solution and is extracted with ethyl acetate. The organic layer is concentrated to afford an orange solid (1.1 g, 78%). M+H⁺=181.8.

¹H NMR (300 MHz) (CDCl₃); δ 7.42 (m, 2H), 8.26 (m, 3H).

### 1b) (4-tert-Butyl-phenyl)(6-fluoro-isoquinolin-1-yl)-amine

1-Chloro-6-fluoro-isoquinoline (1 g, 6.13 mmol) is dissolved in n-BuOH (20 mL) and 4-t-butyl-aniline (1.1 g, 6.74 mmol). 4 N HCl (1 mL) in dioxane (1 mL) is added dropwise. The resulting mixture is heated at 80° C. overnight. The mixture is rotary evaporated, and the residues dissolved in ethyl acetate, washed with saturated NaHCO₃, brine and dried over MgSO₄. The solute is removed and after concentration in vacuo, the organic layer is further purified by silica gel column (hexane 90% to 10% ethyl acetate/hexane) to afford a yellow solid (900 mg, 56%). M+H⁺=295.3.

¹H NMR (300 MHz) (DMSO); δ 1.29 (s, 9H), 7.13 (d, 1H, J=6 Hz), 7.34 (d, 2H, J=8.67 Hz), 7.50 (m, 1H), 7.60 (dd, 1H, J=2.64, 9.8 Hz), 7.72 (d, 2H, 8.67 Hz), 7.96 (d, 1H, 5.65 Hz), 8.61 (dd, 1H, J=5.46, 9.23 Hz), 9.16 (s, 1H).

### 1c) (4-Bromo-6-fluoro-isoquinolin-1-yl)-(4-tert-butyl-phenyl)-amine

(4-tert-Butyl-phenyl)-(6-fluoro-isoquinolin-1-yl)-amine (2.17 g, 7.37 mmol) is dissolved in THF (30 mL). PhMe₃NBr₃ (2.93 g, 7.81 mmol) is added at 0° C. portionwise. The THF is evaporated and the resulting solid is dissolved in methylene chloride and water (200 mL each). The organic layer is washed with water (50 mL, twice) and brine (50 mL, once). The organic phase is separated, dried with Na₂SO₄ and concentrated in vacuo to afford a light brown solid (2.75 g, 99%). M+H⁺=375.2.

¹H NMR (300 MHz) (DMSO); δ 1.29 (s, 9H), 7.36 (d, 2H, J=8.67 Hz), 7.65 (dd, 4H, J=7.35, 8.85 Hz), 8.17 (s, 1H), 8.70 (dd, 1H, J=5.27, 9.42 Hz), 9.38 (s, 1H).

### 1d) (4-tert-Butyl-phenyl)-[6-fluoro-4-(4,4,5,5-tetramethyl-[1,3,2]dioxaborolan-2-yl)-isoquinolin-1-yl]-amine

(4-Bromo-6-fluoro-isoquinolin-1-yl)-(4-tert-butyl-phenyl)-amine (500 mg, 1.34 mmol) is dissolved in DMF (10 mL). Bis(pinocolato)diboron (748 mg, 2.93 mmol) and KOAc (391 mg, 4.019 mmol) are added. The solution is degassed via N₂ for 10 minutes. [(CH₅H₄P(C₆H₅)₂)₂Fe]PdCl₂ is added and heated at 80° C. overnight with stirring. Water (10 mL) is added to the mixture, followed by extraction with ether. The ether layer is passed through a silica gel pad, followed by rotary evaporation to a brown solid. M+H⁺=421.3. The solid is used, without further purification, to prepare Example 1.

### EXAMPLE 2

### [4-(2-Morpholin-4-yl-pyrimidin-4-yl)-isoquinolin-1-yl]-(3-trifluoromethyloxy-phenyl)-amine

To a solution of 1-chloro-4-(2-morpholin-4-yl-pyrimidin-4-yl)-isoquinoline (0.06 g, 1.84×10⁻⁴ m) in n-butanol (30 mL) is added m-trifluoromethoxyaniline (0.10 g, 5.65×10⁻⁴ m) and one drop of concentrated HCl. The mixture is heated to 100° C. for 7 hours and then allowed to cool to room temperature. The mixture is concentrated in vacuo and then dissolved in methylene chloride (75 mL). The organic phase is washed with a saturated solution of sodium bicarbonate, brine, dried over magnesium sulfate and concentrated to a light orange oil. The oil is purified by flash chromatography (SiO₂: hexanes/ethyl acetate). A light yellow oil is collected and crystallized from hexane, m.p. 105-106° C. CHN analysis calc. % C, 61.67; % H, 4.31; % N, 14.98. Found % C, 61.70; % H, 4.64; % N, 14.93.

The starting material is prepared as follows:

### 2a) 2-Thiomethyl-uracil

To a suspension of 2-thiouracil (78.00 g, 0.609 mol) in water (160 mL) and isopropanol (80 mL) cooled to 0-5° C. is added dropwise a 30% sodium hydroxide solution (48.7 g, 1.22 mol: in water 160 mL). A solution of methyl iodide (41.7 mL, 0.669 mol) in isopropanol (150 mL) is added dropwise over 2 hours. The mixture is allowed to warm to room temperature and is stirred for 1 hour. The mixture is concentrated to half volume in vacuo, cooled to 5° C. and then acidified to pH 6.5 with concentrated HCl. The solid precipitate is collected by filtration, washed with cold water and dried in vacuo to give 70 g white solid (81%). M+H¹=142.

¹H NMR (DMSO); δ 12.8 (bs, 1H), 7.90 (d, 1H), 6.07 (d, 1H), 2.37 (s, 3H).

### 2b) 2-Morpholin-4-yl-pyrimidin-4-ol

To the 2-thiomethyluracil (4.0 g, 0.0281 mol) is added morpholine (3.05 g, 0.035 mol). The mixture is heated to 145° C. for 2 hours then cooled to room temperature. The solid is crystallized from ethanol. White needles are collected (2.0 g). Second crop of crystals form approximately 0.50 g (49%). M+H¹=181.

¹H NMR (CDCl₃); δ 12.1 (bs, 1H), 7.85 (d, 1H), 5.79 (d, 1H), 3.75 (m, 8H).

### 2c) 4-(4-bromo-pyrimidin-2-yl)-morpholine

A mixture of 2-morpholin-4-yl-pyrimidin-4-ol (6.08 g, 33.6 mmol) and phosphorus oxybromide (12.5 g, 43.7 mmol) in 330 mL MeCN is heated to reflux for 1 hour. The reaction is cooled to room temperature, concentrated to half volume, and poured over ice. The resulting mixture is neutralized with a saturated solution of NaHCO₃, and then extracted with methylene chloride. The organic phase is washed with saturated NaCl (aqueous), dried over anhydrous MgSO₄, filtered, concentrated and dried to an off-white solid (7.11 g, 87%). M+H¹=245.97.

¹H NMR (CDCl₃); δ 8.05 (d, 1H), 6.70 (d, 1H), 3.75 (m, 8H).

### 2d) 4-Bromo-1-chloro-isoquinoline

To a solution of 4-bromoisoquinoline (52.08 g, 0.250 mol) in methylene chloride (600 mL) is added m-chloroperbenzoic acid (64.47 g, 0.250 mol). The mixture is stirred for 2.5 hours. To the mixture is added 1.5 g of m-chloroperbenzoic acid and the mixture is stirred for 30 minutes. The solution is washed with 1 N NaOH, brine, and then dried over sodium sulfate. The solvent is removed to give a white solid. The solid is crystallized from hot acetone to yield 32.22 g (57.6%) of a white solid. ¹H, ¹³C NMR consistent with structure. The N-oxide (15.75 g, 0.0703 mol) is dissolved in chloroform (50 mL) and cooled in an ice bath. Phosphorus oxychloride (20 mL) is added dropwise and then the mixture is warmed to room temperature and then heated to reflux for 1.5 hours. The mixture is allowed to cool to room temperature and is then poured over ice. The aqueous mixture is neutralized to pH 7-8 with NaHCO₃ and then extracted with chloroform. The organic phase is washed with brine, dried over sodium sulfate and the solvent is removed. The residue is purified by flash chromatography (SiO₂/5% ethyl acetate/hexanes). Collected 12.22 g (72%). M+H¹=389.

¹H NMR; δ 8.50 (s, 1H), 8.40 (d, 1H), 8.20 (d, 1H), 7.92 (t, 1H), 7.79 (t, 1H).

### 2e) 4-Boronic acid-1-chloro-isoquinoline

To a −74° C. solution of 4-bromo-1-chloro-isoquinoline (1.25 g, 5.2 mmol) in anhydrous THF (30 mL) is added dropwise n-BuLi (2.5 M in hexane, 2.3 mL, 5.7 mmol) over 45 minutes. Triisopropyl borate (1.4 mL, 6.1 mmol) is added dropwise, and the mixture is stirred at −74° C. for 2 hours. Upon warming to room temperature, the reaction is quenched with 3 mL water via syringe. After concentrating to an aqueous mixture, the reaction is acidified with 1 N HCl (aqueous) to a pH of approximately 1, which produced a white solid. The solid product is collected by filtration and dried (760 mg, 71%). M+H¹=207.9.

¹H NMR (DMSO-d₆); δ 8.72 (bs, 2H), 8.53 (d, 1H), 8.47 (s, 1H), 8.31 (d, 1H), 7.90 (t, 1H), 7.80 (t, 1H).

### 2f) 1-Chloro-4-(2-morpholin-4-yl-pyrimidin-4-yl)-isoquinoline

4-(4-Bromo-pyrimidin-2-yl)-morpholine (3.7 g, 15.0 mmol) (see Example 2c) and 4-boronic acid-1-chloro-isoquinoline (6.2 g, 29.9 mmol) are dissolved in 60 mL ethylene glycol dimethyl ether (DME) with 3 mL EtOH in a large sealed tube. A solution of Na₂CO₃ (6.1 g, 57.8 mmol) in 20 mL water is added and N₂ is bubbled through the red solution for 5 minutes. The PdCl₂(PPh₃)₂ catalyst (2.1 g, 3.0 mmol) is added, and the mixture is heated to 95° C. for 4.5 hours. Upon cooling, water is added, and the product is extracted with CH₂Cl₂. The organic layer is washed with saturated NaCl (aqeuous), dried over Na₂SO₄, filtered and concentrated. The white solid product (1.8 g, 37%) is obtained using Biotage flash column chromatography silica gel, eluting with 10-20% ethyl acetate in hexane, m.p. 180.5-180.6° C. M+H¹=327.1.

¹H NMR (CDCl₃) δ 8.48 (t, 2H), 8.43. (s, 1H), 8.36 (d, 1H), 7.77 (m; 2H), 6.83 (d, 1H), 3.89 (t, 4H), 3.80 (t, 4H). CHN analysis calc.: % C, 62.48; % H, 4.63; % N, 17.15; % Cl, 10.85. Found: % C, 62.32; % H, 4.58; % N, 16.99; % Cl, 10.81. The solid is used to prepare Example 2.

### EXAMPLE 3

### (4-tert-Butyl-phenyl)-{4-[2-(4-trifluoromethyl-piperidin-1-yl)-pyrimidin-4-yl]-isoquinolin-1-yl}-amine

To a solution of (4-tert-butylphenyl)-[4-(2-chloropyrimidine-4-yl)-isoquinolin-1-yl]amine (0.07 g, 1.80×10⁻⁴ m) in n-butanol (30 mL) is added 4-trifluoromethylpiperidine (0.07 g, 4.57×10⁻⁴ m) and triethylamine (0.50 mL). The mixture is heated to 100° C. for 16 hours and then allowed to cool to room temperature. The mixture is concentrated in vacuo and then dissolved in methylene chloride (75 mL). The organic phase is washed with a saturated solution of sodium bicarbonate, brine, dried over magnesium sulfate and concentrated to a oil. The oil is purified by flash chromatography (SiO₂: 75% hexanes/25% ethyl acetate). A light yellow oil is collected and crystallized from ether, m.p. 179-180° C. CHN analysis calc. % C, 68.89; % H, 5.98; % N, 13.85. Found % C, 68.91; % H, 5.73; % N, 13.73.

### 3a) (4-tert-Butyl-phenyl)-isoquinolin-1-yl-amine

A 1 L, round bottom flask with a magnetic stirrer is charged with 100 mL of n-butanol and 9.5 mL (110.2 mmol) of concentrated HCl. To this is added 2-chloroisoquinoline (15.01 g; 91.74 mmol) and 16.6 mL of 4-tert-butyl aniline (14.94 g, 100.1 mmol). The mixture is heated to 70° C. for 3 hours. The n-butanol is evaporated in vacuo and the resulting syrupy mixture is mixed with pentane. The resulting white solid is filtered off and dried. The solid is dissolved in ethyl acetate and dichloromethane and made slightly basic with sodium bicarbonate. The organic layer is dried and concentrated to afford (4-tert-butyl-phenyl)isoquinolin-1-yl-amine as a whitish solid weighing 20 g (78.9%). MS 277.2 m+1 (100%).

¹H NMR (DMSO); δ 8.08 (d, 1H), 7.90 (d, 3H), 7.72 (d, 1H), 7.57 (m, 4H), 7.37 (d, 2H), 1.32 (s, 9H).

### 3b) (4-Bromo-isoquinolin-1-yl)-(4-tert-butyl-phenyl)-amine

In a 1 L, round bottom flask, (4-tert-butyl-phenyl)-isoquinolin-1-yl-amine (18.7 g, 67.7 mmol) is mixed with 100 mL of THF and cooled in an ice bath. To this is added, dropwise over 2 hours, phenyltrimethylammonium tribromide (25.12 g, 66.47 mmol) dissolved in 200 mL of THF. The reaction is allowed to rise to room temperature overnight. The reaction mixture is poured into 2 L of hexane with stirring. The solid is filtered, dried and dissolved in dichloromethane. The solution is washed with 2×250 mL of saturated sodium bicarbonate solution, followed by 1×250 mL of water. The organic solution is dried and concentrated. The solid is mixed with hexane, filtered and dried, affording 19.8 g (82.3%) of (4-bromo-isoquinolin-1-yl)-(4-tert-butyl-phenyl)-amine as a off yellow solid. MS 355 M+(100%).

¹H NMR (DMSO); δ 8.23 (s, 1H), 8.08 (d, 1H), 7.73 (t, 1H), 7.56 (m, 3H), 7.38 (d, 2H), 7.07 (bs, 1H), 1.33 (s, 9H).

### 3c) (4-boronoic acid-isoquinolin-1-yl)-(4-tert-butylphenyl)-amine

To a −74° C. solution of (4-bromo-isoquinolin-1-yl)-(4-tert-butyl-phenyl)amine (10.3 g, 29.0 mmol) in anhydrous THF (130 mL) is added dropwise n-BuLi (2.5 M in hexane, 30.0 mL, 75.0 mmol) over 1 hour. Triisopropyl borate (8.0 mL, 34.7 mmol) is added dropwise, and the mixture is stirred at −74° C. for 4.5 hours. Upon warming to room temperature, the reaction is quenched with 20 mL water via syringe. After concentrating to an aqueous mixture, the reaction is acidified with 1 N HCl (aqueous) to a pH of approximately 1, to produce a white solid. The solid product is collected by filtration and dried (6.74 g, 73%).

¹H NMR (DMSO-d₆); δ 11.59 (s, 1H), 8.95 (d, 1H), 8.72 (d, 1H), 8.61 (broad s), 8.01 (t, 1H), 7.82 (t, 1H), 7.76 (s, 1H), 7.62 (d, 2H), 7.50 (d, 2H), 1.36 (s, 9H); MS 321.3 m/z (M+H).

### 3d) (4-tert-Butyl-phenyl)-[4-(2-chloropyrimidin-4-yl)-isoquinolin-1-yl]-amine

2,4-Dichloropyrimidine (1.54 g, 10.3 mmol) and (4-boronic acid-isoquinolin-1-yl)-(4-tert-butyl-phenyl)-amine (3.00 g, 9.37 mmol) are combined in 45 mL ethylene glycol DME in a large sealed tube. The PdCl₂(PPh₃)₂ catalyst (0.66 g, 0.94 mmol) and a 3.0 M aqueous solution of Na₂CO₃ (12.5 mL, 37.5 mmol) are added and N₂ is bubbled through the solution for 5 minutes. The reaction mixture is then heated to 85-90° C. for 2.5 hours. Upon cooling, the solvent is removed and water (15 mL) is added to the mixture. The product is extracted with CH₂Cl₂ (3×200 mL) washed with saturated NaCl (aqueous), (3×200 mL) dried over MgSO₄, filtered and concentrated. The crude product is purified by flash column chromatography (15-20% ethyl acetate in hexane) and re-crystallized from EtOAc/hexane to give a pure green product (1.81 g, 4.66 mmol) in 50% yield: m.p. 257.3-258.4° C. CHN analysis calc. % C, 71.04; % H, 5.44; % N, 14.41; % Cl, 9.12. Found % C, 70.80; % H, 5.60; % N, 14.35; % Cl, 8.99.

¹H NMR (DMSO-d₆); δ 9.58 (s, 1H), 8.80 (d, 1H), 8.64 (d, 1H), 8.50 (d, 1H), 8.35 (s, 1H), 7.90 (d, 1H), 7.82 (t, 1H), 7.76 (d, 2H), 7.71 (t, 1H), 7.39 (d, 2H), 1.31 (s, 9H); MS 389.2, 387.3 m/z (M+H, M−H).

### EXAMPLE 4

### [4,7′]Biisoquinolinyl-1-yl-(4-tert-butyl-phenyl)-amine

A suspension of 1-chloro-[4,7]biisoquinolinyl (5.0 g, 17.2 mmol), 4-tert-butylaniline (4.0 mL, 1.5 eq.) and 4.0 M HCl/dioxane (6.45 mL/1.5 eq.) in EtOH (100 mL) is stirred for 20 hours in a sealed tube at 80° C. The reaction mixture is then cooled and concentrated to give a yellow oil. The oil is taken up in EtOAc and neutralized with 3 N NaOH. The organic phase is separated, dried (MgSO₄) and concentrated to give the crude material. The crude product is purified by silica gel. Eluted with 9:1 hexane/EtOAc then 4:1 hexane/EtOAc. Pure product is isolated as a yellow solid, 4.5 g (65%); m.p. 217-219° C.

¹H NMR (DMSO-d₆) δ 9.40 (s, 1H), 9.31 (s, 1H), 8.67 (d, 1H), 8.57 (d, 1H), 8.24 (s, 1H), 8.09 (d, 1H), 8.04 (s, 1H), 7.92 (m, 2H), 7.80 (m, 2H), 7.73 (m, 2H), 7.37 (d, 2H), 1.31 (s, 9H); MS 404.21 m/z (M+H).

### 4a) 1-Chloro-[4,7′]biisoquinolinyl

A solution of 4-bromo-1-chloroisoquinoline (see Example 2d) (5.0 g, 20.7 mmol) in 150 mL THF is cooled to −78° C. A solution of n-BuLi (1.6 M in hexanes) (15 mL, 24 mmol) is added dropwise and the reaction temperature is maintained at −78° C.˜−68° C. The reaction mixture is kept stirring at −78° C. for 30 minutes. ZnBr₂ (dried under vacuum at 80° C.) (6.5 g, 24.9 mmol) is dissolved in 50 mL THF and is transferred to above mixture slowly at −78° C. The solution is stirred 40 minutes at −78° C., then warmed to room temperature by removing the cooling bath. Pd(PPh₃)₄ (2.4 g, 2.1 mmol is added followed by trifluoromethanesulfonic acid isoquinolin-7-yl ester-(5.7 g, 20.7 mmol) in 50 mL THF. The reaction mixture is heated to 60° C. for 30 minutes and is then concentrated. The resulting oil is dissolved in dichloromethane and washed with saturated NaHCO₃. Organic phase is separated, dried (MgSO₄) and concentrated to give a yellow solid. The solid is collected by filtration, washed with ether then hexane and dried under vacuum. 5.68 g (94%) yellow solid is obtained. m.p. 169.0-169.6° C.

¹H NMR (400 MHz, DMSO-d₆) d 9.44 (s, 1H); 8.61 (d, 1H, J=5.6 Hz); 8.45-8.43 (m, 1H); 8.40 (s, 1H); 8.34 (s, 1H); 8.18 (d, 1H, J=8.1 Hz); 7.97-7.90 (m, 5H) ppm. API-MS, m/z 291.14 ([M+H]⁺, calcd. 291.06).

### EXAMPLE 5

### 2-{4-[1-(4-tert-Butyl-phenylamino)-isoquinolin-4-yl]-pyrimidin-2-ylamino}-ethanol

(4-tert-Butyl-phenyl)-[4-(2-chloropyrimidin-4-yl)-isoquinolin-1-yl]-amine 9 (see Compound 3d) (20 mg, 0.0515 mmol) and 2-hydroxylethylamine (50 mg) are dissolved in n-butanol and heated to 80° C. in a sealed tube for 16 hours. Ten (10) mL DCM is added and the solution is washed with NH₄Cl (10 mL), water and brine. Organic chromatography (SiO₂, 10-60% EtOAc-hexanes gradient elution) provides product (21 mg, 99%).

¹H NMR (300 MHz, CD₃OD); δ 8.31 (d, J=9.0 Hz, 1H), 8.21 (d, J=6.0 Hz, 1H), 7.98 (s, 1H), 7.62 (t, J=6.0 Hz, 1H), 7.53 (t, J=6.0 Hz, 1H), 7.45 (d, J=9.0 Hz, 2H), 7.31 (d, J=9.0 Hz, 2H), 6.77 (d, J=6.0 Hz, 1H), 3.64 (t, J=6.0 Hz, 1H), 3.47 (t, J=6.0 Hz, 1H), 1.24 (s, 9H). HRMS ESI m/z 414.2277 (M+H⁺, C₂₅H₂₇ON₅ requires 414.2294). HPLC, C¹⁸ reverse phase column, gradient 10-90% MeCN—H₂O UV −250 nM, retention time 9.41 minutes.

### EXAMPLE 6

### (4-tert-Butyl-phenyl)-(4-quinazolin-6-yl-isoquinolin-1-yl)-amine

NaH (60%) (0.62 g, 15.52 mmol) is added to a solution of 4-tert-butyl-aniline (1.16 g, 7.76 mmol) in 75 mL of THF in a sealed tube and stirred at room temperature for 30 minutes. 6-(1-Chloro-isoquinolin-4-yl)-quinazoline (1.5 g, 5.18 mmol) is added. The reaction mixture is heated to 80° C. for 4 hours. Then it is quenched with water. The reaction mixture is extracted with DCM. The combined organic phase is dried over sodium sulfate and concentrated. The residue is re-crystallized with DCM and EtOAc to give 0.8 g yellow solid. The mother liquor is further purified chromatography (25-50% EA/H) to give 0.5 g yellow solid. Both solid is proved by NMR to be desired product. Yield is 62%. The product is characterized by NMR, MS, m.p.

¹H NMR (DMSO, 500 MHz); δ 9.68 (s, 1H), 9.35 (s, 1H), 9.32 (s, 1H), 8.66 (d, J=8.4771 Hz, 1H), 8.28 (s, 1H), 8.18-8.13 (m, 2H), 9.05 (s, 1H), 7.82-7.18 (m, 3H), 7.38 (s, 2H), 1.31 (s, 9H); m.p. 213-214.5° C. API-MS m/z 405.15 ([M+H]⁺, calcd. 405.20).

### 6a) 6-Iodoquinazoline

To a solution of quinazoline (2.1 g, 16.13 mmol) in concentrated H₂SO₄ (16 mL) is added NIS (5.4 g, 24 mmol) at 0° C. The reaction mixture is stirred for 10 minutes then warmed up to room temperature and stirred for 15 hours. Further NIS (2.0 g. 8.9 mmol) is added and the mixture is stirred for another 5 hours. The reaction mixture is poured onto crushed ice (80 g) and stirred for 1 hour. The solution is filtered, the pH is adjusted to 7 with concentrated aqueous NH₃ and stirred for another 1 hour at 0° C. after which it is filtered and washed with ice-cold water. The solid is air dried and yields 3.4 g (87%) of the desired product.

¹H NMR (400 MHz, DMSO); δ 9.57 (s, 1H), 9.43 (s, 1H), 8.65 (s, 1H), 8.29 (d, J=8.0 Hz, 1H), 7.81 (d, J=8.0 Hz, 1H), MS ESI m/z 256 M+H⁺, C₆H₃IN₄.

### 6b) 6-(1-Chloro-isoquinolin-4-yl)quinazoline

A solution of 4-bromo-1-chloro-isoquinoline (see Compound 2d) (4.74 g, 19.52 mmol) in 300 mL THF is cooled to −72° C. A solution of n-BuLi (2.5 M in hexanes) (9.37 mL, 23.42 mmol) is added dropwise and the reaction temperature maintained at −70° C.˜−68° C. for 30 minutes. ZnBr₂ (4.84 mg, 21.47 mmol) is dissolved in 50 mL THF and is transferred to above mixture slowly at −70° C. The solution is stirred 20 minutes at −70° C., then warmed to room temperature. Pd(PPh₃)₄ (2.25 g, 1.95 mmol) and 6-iodoquinazoline (5 g, 19.52 mmol) in 4 mL THF are added to the reaction mixture dropwise in the order. Then the reaction mixture is heated to 60° C. for 30 minutes, then kept at room temperature overnight. The reaction mixture is quenched with NH₄Cl and extracted with ethyl acetate. White solid (4.0 g) is collected by filtration. The organic solution is washed with saturated NH₄Cl, then brine and dried over sodium sulfate. The solution is concentrated until white solid comes out from solution. The solid is collected by filtration, washed with ether and dried under vacuum. 1.53 g solid is obtained. Yield is 97.6%.

¹H NMR (DMSO, 400 MHz); δ 9.73 (s, 1H), 9.40 (s, 1H), 8.48-8.44 (m, 1H), 8.42 (s, 1H), 8.40 (s, 1H), 8.21 (s, 3H), 7.96-7.9 (m, 3H). API-MS m/z 292.02 ([M+H]⁺, calcd. 292.06).

### EXAMPLE 7

### [4,7′]Biisoquinolinyl-1-yl-(2-tert-butyl-pyrimidin-5-yl)-amine

NaH (60% in oil) (0.60 g, 15.2 mmol) is added to a solution of 5-amino-2-tert-butylpyrimidine (1.14 g, 7.6 mmol) in 75 mL of THF in a sealed tube and stirred at room temperature for 30 minutes. 1-Chloro-[4,7′]biisoquinolinyl (see Compound 4a) (2.0 g, 6.9 mmol) is then added in a single portion. The reaction is heated at 110° C. for 18 hours whereupon it was cooled, quenched with water and the volatiles removed in vacuo. The residue is dissolved into DCM and washed with water followed by brine. The organic phase is dried over sodium sulfate and the volatiles removed in vacuo. The residue is purified by silica gel chromatography (25-50% EtOAc in hexanes) to give 1.53 g (55%) pale yellow solid; m.p. 231.1-232.0° C.

¹H NMR (300 MHz, CDCl₃); δ 9.34 (s, 1H); 9.19 (s, 2H); 8.60 (d, 1H, J=5.7 Hz); 8.09-8.13 (m, 3H); 7.98 (d, 1H, J=8.3 Hz); 7.83-7.91 (m, 2H); 7.77 (d, 1H, J=5.7 Hz); 7.68-7.71 (m, 2H); 7.15 (s, 1H); 1.48 (s, 9H) ppm. API-MS, m/z 406.15 ([M+H]⁺, calcd. 406.19).

### EXAMPLE 8

### (4-tert-Butyl-2-fluoro-phenyl)-[4-(2-morpholin-4-yl-pyrimidin-4-yl)-isoquinolin-1-yl]-amine

4-tert-Butyl-2-fluoro-phenylamine is coupled to 1-chloro-4-(2-morpholin-4-yl-pyrimidin-4-yl)-isoquinoline as described in Example 2. API-MS, m/z 458.50 ([M+H]⁺, calcd. 458.22).

¹H NMR (300 MHz, CDCl₃) δ 8.51 (d, 1H, J=7.54 Hz), 8.42 (m, 2H), 8.36 (s, 1H), 8.02 (d, 1H, J=7.91 Hz), 7.70 (m, 1H), 7.63 (m, 1H), 7.46 (d, 1H, J=3.01 Hz), 7.21 (m, 2H), 6.84 (d, 1H, J=5.27 Hz), 3.89 (m, 4H), 3.80 (t, 4H, J=4.71 Hz) 1.33 (s, 9H).

### 8a) N-(4-tert-Butyl-2-fluoro-phenyl)-acetamide

A solution of N-(4-tert-butyl-phenyl)-acetamide (191 mg, 1 mmol) and 1-chloromethyl-4-fluoro-1,4-diazoniabicyclo[2.2.2]octane bis-(tetrafluoroborate) (355 mg, 1 mmol) in acetonitrile (10 mL) is refluxed under a N₂ atmosphere for 16 h. Reaction is cooled and volatiles removed in vacuo. The residue is diluted with CH₂Cl₂ (20 mL) and washed with H₂O (10 mL), sat. NaHSO₄ (10 mL), brine (10 mL) and dried over Na₂SO₄. Drying agent is filtered and the volatiles removed in vacuo. The residue is purified by silica gel chromatography (5% EtOAc in Hexanes) to yield 70 mg of N-(4-tert-butyl-2-fluoro-phenyl)-acetamide as a white crystalline material; mp 163.5-164.7° C.

¹H NMR (300 MHz, CDCl₃) δ 8.02 (t, 1H, J=8.02 Hz), 7.46 (br s, 1H), 7.05-6.97 (m, 2H), 2.12 (s, 3H), 1.20 (s, 9H).

### 8b) 4-tert-Butyl-2-fluoro-phenylamine

N-(4-tert-Butyl-2-fluoro-phenyl)-acetamide (70 mg, 0.33 mmol) is dissolved in EtOH (2 mL) with 1N HCl (10 ml, 0.01 mmol) and heated to reflux for 72 hr. The reaction is cooled to rt and the volatiles removed in vacuo. The remaining aqueous solution is washed 1×5 mL Et₂O, made basic with sat. NaHCO₃, and extracted 3×5 mL CH₂Cl₂. Organic extracts are combined and dried over Na₂SO₄. Volatiles are removed to yield 30 mg (54%) product 4-tert-Butyl-2-fluoro-phenylamine as a straw colored oil.

¹H NMR (300 MHz, CDCl₃) δ 7.00 (m, 1H), 6.94 (m, 1H), 6.71 (m, 1H), 3.59 (br s, 2H), 1.26 (s, 9H).

### EXAMPLE 9

### (6-tert-Butyl-pyridin-3-yl)-[4-(2-morpholin-4-yl-pyrimidin-4-yl)-isoquinolin-1-yl]-amine

6-tert-Butyl-pyridin-3-ylamine is coupled to 1-chloro-4-(2-morpholin-4-yl-pyrimidin-4-yl)-isoquinoline as described in Example 2: API-MS, m/z 441.44 ([M+H]⁺, calcd. 441.23).

¹H NMR (300 MHz, CDCl₃) δ 8.72 (d, 1H, J=2.64 Hz), 8.51 (d, 1H, J=7.91 Hz), 8.43 (d, 1H, J=4.90 Hz), 8.32 (s, 1H), 8.24 (dd, 1H, J=8.48, 2.83 Hz), 8.03 (d, 1H, J=8.29 Hz), 7.72 (t, 1H, J=7.16 Hz), 7.63 (t, 1H, J=6.97 Hz), 7.39 (d, 1H, J=8.67 Hz), 7.28 (s, 1H), 6.83 (d, 1H, J=4.90 Hz), 3.84 (m, 8H), 1.40 (s, 9H).

### 9a) N′-(6-tert-Butyl-pyridin-3-yl)-hydrazinium hydrochloride

A solution of N′-(6-tert-Butyl-pyridin-3-yl)-hydrazinebiscarboxylic acid tert-butyl ester (Tet. Lett. 2000, 41, 3025-3028) (2.45 g, 6.7 mmol) in isopropanol (100 mL) and 4.0M HCl/dioxane (16.7 mL, 67 mmol) is heated to reflux for 18 hours, then cooled and triturated with ether (200 mL). The precipitated product is filtered, washed with 25 mL anhydrous ether and dried to yield 1.2 g (88%) of the title compound as a pale yellow solid; mp 210.1-212.6° C. API-MS, m/z 166.18 ([M+H]⁺, calcd. 166.13).

¹H NMR (400 MHz, DMSO-d₆) δ 8.45 (d, 1H, J=2.53 Hz), 8.08 (dd, 1H, J=9.10, 2.53 Hz), 7.93 (d, 1H, J=9.1 Hz), 1.44 (s, 9H).

### 9b) 6-tert-Butyl-pyridin-3-ylamine

Zinc dust (3.13 g, 48 mmol) is added in a single portion to a solution of N′-(6-tert-butyl-pyridin-3-yl)-hydrazinium hydrochloride (1.2 g, 6.0 mmol) in methanol (30 mL) and 4M HCl/dioxane (12 mL, 48 mmol) and the solution stirred at rt for two days until the starting material hydrazine is consumed. Volatiles are removed via rotovap and the residue treated with 40 mL 28% aqueous ammonia. The product is then extracted into ether (3×30 mL), shaken with brine, dried over Mg₂SO₄ and filtered. Volatiles are removed to yield 0.802 g (89%) product as an orange solid; mp 61.5-62.7° C. API-MS, m/z 151.16 ([M+H]⁺, calcd. 151.11).

¹H NMR (300 MHz, CDCl₃) δ 8.07 (d, 1H, J=2.64 Hz), 7.12 (d, 1H, J=8.29 Hz), 6.94 (dd, 1H, J=8.48, 2.83 Hz), 3.55 (s, 2H), 1.32 (s, 9H).

### EXAMPLE 10

### [4,7′]Biisoquinolinyl-1-yl-(6-tert-butyl-pyridin-3-yl)-amine

6-tert-Butyl-pyridin-3-ylamine is coupled to 1-Chloro-[4,7′]biisoquinolinyl as described in Example 7. API-MS, m/z 405.17 ([M+H]⁺, calcd. 405.20).

¹H NMR (300 MHz, CDCl₃) δ 9.32 (s, 1H), 8.80 (s, 1H), 8.59 (d, 1H, J=5.65 Hz), 8.33 (d, 1H, J=5.63 Hz), 8.13 (s, 2H), 8.08 (s, 1H), 7.96 (d, 1H, J=8.69), 7.85 (m, 2H), 7.75 (d, 1H, J=5.65 Hz), 7.67 (m, 2H), 7.41 (d, 1H, J=8.67), 1.41 (s, 9H).

### EXAMPLE 11

### [4,7′]Biisoquinolinyl-1-yl-(5-isopropenyl-pyridin-2-yl)-amine

5-Isopropenyl-pyridin-2-ylamine is reacted with 1-chloro-[4,7′]biisoquinolinyl as described in example 7.

### 11a) 2-(6-Fluoro-pyridin-3-yl)-propan-2-ol

A solution of 5-bromo-2-fluoro-pyridine (5.0 g, 28.4 mmol) in ether (300 mL) is cooled to −78° C. and a solution of 2.5M n-BuLi in hexanes (11.9 mL, 29.8 mmol) is added dropwise with stirring. The reaction is stirred for a further 15 minutes whereupon acetone (10.4 mL, 142 mmol) is added dropwise. Reaction is warmed to rt and quenched with sat. NH₄Cl (5 mL). The reaction is washed with sat. NH₄Cl (50 mL), H₂O (50 mL), brine (50 mL) and dried over Mg₂SO₄. Drying agent is filtered and the volatiles removed in vacuo to yield 4.1 g (93%) product 2-(6-fluoro-pyridin-3-yl)-propan-2-ol as a clear oil.

¹H NMR (300 MHz, CDCl₃) δ 8.32 (d, 1H, J=2.26), 7.93 (m, 1H), 6.89 (dd, 1H, J=8.85, 2.83), 1.26 (s, 6H).

### 11b) 2-Fluoro-5-isopropenyl-pyridine

A solution of 2-(6-fluoro-pyridin-3-yl)-propan-2-ol (8.3 g, 53.5 mmol) and p-TSA monohydrate (0.46 g, 2.7 mmol) in toluene (500 mL) is refluxed with a Dean-Stark trap and a condenser until the theoretical amount of water is collected. The reaction is then cooled and extracted with sat. NaHCO₃ (3×50 mL). Volatiles are removed to yield 7.30 g (99%) product as a straw colored oil.

¹H NMR (300 MHz, CDCl₃) δ 8.21 (d, 1H, J=2.53), 7.77 (m, 1H), 6.81 (dd, 1H, J=8.34, 2.78), 5.28 (s, 1H), 5.09 (s, 1H), 2.08 (s, 3H).

### 11c) 5-Isopropenyl-pyridin-2-ylamine

A solution of 2-fluoro-5-isopropenyl-pyridine (6.3 g, 45.9 mmol) in dioxane (30 mL) and conc. ammonium hydroxide (178 mL, 1.37 mol) is heated in a glass bomb at 150° C. for 48 hr. The reaction is then cooled and extracted with Et₂O (3×100 mL). Extracts are combined, washed with brine (100 mL), dried over Mg₂SO₄, and filtered. Volatiles are removed and the residue is purified by silica gel chromatography (50% EtOAc in Hexanes). Product containing fractions are combined and volatiles are removed in vacuo to yield 3.6 g (58%) product as a colorless oil. API-MS, m/z 135.14 ([M+H]⁺, calcd. 135.08).

¹H NMR (300 MHz, CDCl₃) δ 8.19 (d, 1H, J=1.88), 7.58 (dd, 1H, J=8.67, 2.26), 6.47 (d, 1H, J=8.67), 5.25 (s, 1H), 4.46 (br s, 2H), 2.10 (s, 3H).

### EXAMPLE 12

### [4,7′]Biisoquinolinyl-1-yl-(5-isopropyl-pyridin-2-yl)-amine

5-Isopropyl-pyridin-2-ylamine is reacted with reacted with 1-chloro-[4,7′]biisoquinolinyl as described in example 7.

### 12a) 5-Isopropyl-pyridin-2-ylamine

To solution of 5-isopropenyl-pyridin-2-ylamine (1.2 g, 8.9 mmol) in ethanol (30 mL) is added 100 mg 10% Pd/C and the resulting suspension is stirred vigorously under a hydrogen atmosphere (1 atm) for 18 hr. The reaction is then filtered through celite and the volatiles removed to yield 1.0 g (82%) product as a colorless oil. API-MS, m/z 137.14 ([M+H]⁺, calcd. 137.10).

¹H NMR (300 MHz, CDCl₃) δ 7.94 (d, 1H, J=2.26); 7.32 (dd, 1H, J=8.48, 2.45); 6.47 (d, 1H, J=7.91); 4.27 (br s, 2H); 4.46 (br s, 2H); 2.81 (m, 1H); 1.21 (d, 6H, J=6.78).

### EXAMPLE 13

### 4-(2-Morpholin-4-yl-pyrimidin-4-yl)-isoquinolin-1-ylamine

A bomb is charged with 1-chloro-4-(2-morpholin-4-yl-pyrimidin-4-yl)-isoquinoline (658 mg, 2.0 mmol), conc. NH₄OH (10 mL) and dioxane (10 mL). The bomb is sealed and heated to 120° C. for 24 hours. When cool, the reaction mixture is reduced in volume and mixed with water, filtered and the solid dried under high vacuum. Yield 541 mg (88%); mp 254.8-255.8° C.

¹³C NMR (100 MHz, CDCl₃)δ 165.31, 161.62, 158.97, 158.42, 144.74, 134.53, 130.79, 125.94, 124.66, 124.59, 118.80, 116.79, 109.86, 66.37, 44.36.

### EXAMPLE 14

### 4-Methoxy-N-[4-(2-morpholin-4-yl-pyrimidin-4-yl)-isoquinolin-1-yl]-benzamide

A solution of 4-(2-Morpholin-4-yl-pyrimidin-4-yl)-isoquinolin-1-ylamine and 4-methyl morpholine (36 μL, 0.327 mmol) in 80% THF/DMA is cooled in an ice bath and p-anisoyl chloride (46 μL, 0.326 mmol) is added dropwise. The reaction mixture is allowed to warm to rt, mixed with water, extracted with CH₂Cl₂, dried over Na₂SO₄ and filtered. The residue is purified by flash chromatography using 100% CH₂Cl₂ to 20% EtOAc/CH₂Cl₂. Yield 14.6 mg (10.1%) MS 442.15 M+1, 440.16 M−1.

### EXAMPLE 15

### (4-tert-Butyl-phenyl)-[4-(4-morpholin-4-yl-quinazolin-6-yl)-isoquinolin-1-yl]-amine

A microwave reaction vial is charged with (4-boronic acid-isoquinolin-1-yl)-(4-tert-butylphenyl)-amine (120.9 mg, 0.38 mmol, 1.2 eq), K₂CO₃ (128.7 mg, 0.93 mmol, 3 eq), 6-bromo-4-morpholin-4-yl-quinazoline (92.5 mg, 0.31 mmol, 1 eq) and 4:1 DME:H₂O (5 mL). N₂ gas is bubbled through this mixture. PdCl₂(PPh₃)₂ (47.8 mg, 0.068 mmol, 0.22 eq) is added and the vial sealed. This is heated to 120° C. for 30 min under microwave heating. The residue is mixed with CH₂Cl₂ and washed with brine. The organics are dried over Na₂SO₄, filtered and concentrated, The residue is purified by flash chromatography using EtOAc/CH₂Cl₂. Yield 40.1 mg (26.4%) 490.4 M+1, 488 M−1,

¹³C NMR (75 MHz, CDCl₃) δ 163.58, 153.04, 151.60, 149.99, 145.16, 140.5, 136.23, 134.63, 134.17, 129.34, 127.5, 125.69, 124.92, 124.40, 124.07, 123.5, 121.0, 119.71, 17.18, 115.82, 65.72, 49.70, 49.26, 33.33, 30.41.

### EXAMPLE 16

### 4-tert-Butyl-phenyl)-[4-(2-methylamino-pyrimidin-4-yl)-isoquinolin-1-yl]-amine

A solution of (4-tert-Butyl-phenyl)-[4-(2-chloropyrimidin-4-yl)-isoquinolin-1-yl]-amine (20 mg, 0.0515 mmol), MeNH₂HCl (4.2 mg, 1.2 eq) and Et₃N (10 mg) in n-butanol is heated at 80° C. in a sealed tube for 16 h. The reaction mixture is diluted with CH₂Cl₂ (10 mL) and the solution is washed with NaHCO₃ (10 mL), H₂O (10 mL) and brine (10 mL). The organic layer is dried (Mg₂SO₄) and concentrated to an oil. Chromatography (SiO₂, 10-60% EtOAc-hexanes gradient elution) provided product (21 mg, 99%). HRMS ESI m/z 384.2158 (M+H⁺, requires 384.2188).

¹H NMR (300 MHz, CD₃OD) δ 8.44 (d, J=6.0 Hz, 1H), 8.32 (d, J=6.0 Hz, 1H), 8.25 (s, 1H), 7.91 (d, J=9.0 Hz, 1H), 7.62 (t, J=6.0 Hz, 1H), 7.53 (d, J=9.0 Hz, 2H), 7.51 (m, 1H), 7.34 (d, J=9.0 Hz, 2H), 6.75 (d, J=3.0 Hz, 1H), 5.16 (bro, 1H), 2.99 (d, J=3.0 Hz, 2H), 1.27 (s, 9H).

### EXAMPLE 17

### (4-tert-Butyl-phenyl)-[4-(2-methylsulfanyl-pyrimidin-4-yl)-isoquinolin-1-yl]-amine

A solution of 3c (4-boronic acid-isoquinolin-1-yl)-(4-tert-butylphenyl)-amine (1.1 g, 1.5 eq), 4-chloro-2-methylsulfanyl-pyrimidine (161 mg, 1.0 eq) and Pd(PPh₃)₄ (0.1 eq) in DME (10 mL) was treated with Na₂CO₃ (2M, 5 mL) is reflux for 1.5 h. The solution is cooled to rt and the DME was removed in vacuo. The residue is treated with CH₂Cl₂ and H₂O. The organic extracts are combined, dried (MgSO₄) and concentrated to an oil. Chromatography (SiO₂, 40% EtOAc-hexane) provided (4-tert-butyl-phenyl)-[4-(2-methylsulfanyl-pyrimidin-4-yl)-isoquinolin-1-yl]-amine (600 mg, 65%) as light yellow solid. MS ESI m/z 368.20 (M+H).

¹H NMR (300 MHz, CDCl₃) δ 8.34 (d, J=6.0 Hz, 1H), 8.38 (d, J=9.0 Hz, 1H), 8.29 (d, J=9.0 Hz, 1H), 8.08 (s, 1H), 7.69 (t, J=6.0 Hz, 1H), 7.61 (d, J=9.0 Hz, 1H), 7.54 (d, J=6.0 Hz, 2H), 7.49 (d, J=3.0 Hz, 1H), 7.38 (d, J=6.0 Hz, 1H), 2.73 (bro, 3H), 1.32 (s, 9H).

### EXAMPLE 18

### [4-(4-Benzyloxy-quinazolin-6-yl)-isoquinolin-1-yl]-(2-tert-butyl-pyrimidin-5-yl)-amine

NaH (18 mg, 0.66 mmol, 4 eq) is added to a solution of 5-amino-2-tert-butylpyrimidine (37 mg, 0.25 mmol, 1.5 eq) in THF (1 mL) and the resulting suspension is stirred for 30 min. 4-Benzyloxy-6-(1-chloro-isoquinolin-4-yl)-quinazoline (65 mg, 0.164 mmol, 1 eq) is added to this solution and heated to 80° C. for 4 h. CH₂Cl₂ (5 mL) is added and the solution is washed with H₂O and brine. Chromatography (SiO₂, 10-60% EtOAc-hexanes gradient elution) provides product [4-(4-benzyloxy-quinazolin-6-yl)-isoquinolin-1-yl]-(2-tert-butyl-pyrimidin-5-yl)-amine (39 mg, 46%). MS/ESI, M+1=513.19

¹H NMR (400 MHz, CDCl₃) δ 9.18 (s, 2H), 8.81 (s, 1H), 8.37 (d, J=8.0 Hz, 1H), 8.04 (d, J=8.0 Hz, 2H), 7.98 (s, 1H), 7.90 (d, J=8.0 Hz, 1H), 7.66 (m, 2H), 7.57 (m, 1H), 7.51 (d, J=4.0 Hz, 2H), 7.38 (d, J=8.0 Hz, 2H), 7.35 (m, 1H), 5.60 (s, 2H), 1.43 (s, 9H).

### 18a) 4-Benzyloxy-6-iodo-quinazoline

NaH (152 mg, 5.7 mmol, 1.5 eq) is added to a solution of benzyl alcohol (820 mg, 7.6 mmol, 2 eq) in DMF (10 mL) an the resulting suspension is stirred for 30 min. Then 4-chloro-6-iodo-quinazoline-(1.1 g, 3.8 mmol, 1 eq) is added to this solution and the reaction mixture is heated to 100° C. for 4 h. CH₂Cl₂ (50 mL) is added and the solution is washed with H₂O and brine. Chromatography (SiO₂, 10-60% EtOAc-hexanes gradient elution) provides 4-benzyloxy-6-iodo-quinazoline (1.21 g, 88%). MS/ESI, M+1=362.88

### 18b) 4-Benzyloxy-6-(1-chloro-isoquinolin-4-yl)-quinazoline

A solution of 4-bromo-1-chloro-isoquinoline (802 mg, 3.31 mmol) in THF (20 mL) is cooled to −72° C. A solution of n-BuLi (2.5 M in hexanes) (1.6 mL, 3.97 mmol) is added dropwise and the reaction temperature maintained at −70° C. for 30 min. ZnBr₂ (900 mg, 4.2 mmol) is dissolved in THF (6 mL) and is transferred to above mixture slowly at −70° C. The solution is stirred 40 min at −70° C., then warmed to room temperature. Pd(PPh₃)₄ (400 mg, 0.36 mmol) in THF (6 mL) and 4-benzyloxy-6-iodo-quinazoline (1.2 g, 3.31 mmol) in THF (4 mL) are added to the reaction mixture dropwise. The solution is heated to 60° C. for 30 min, then kept at rt overnight. The reaction mixture is diluted with ethyl acetate, washed with sat. NH₄Cl, then brine, and dried over sodium sulfate. The solution is concentrated until white solid precipitates from solution. The solid is collected by filtration, washed with ether and dried under vacuum. 600 mg of 4-benzyloxy-6-(1-chloro-isoquinolin-4-yl)-quinazoline is obtained. Yield was 46%. MS/ESI+, M+1=397.96

### EXAMPLE 19

### (4-Methylsulfanyl-phenyl)-[4-(6-morpholin-4-yl-pyrazin-2-yl)-isoquinolin-1-yl]-amine

NaH (11 mg, 0.4 mmol, 4 eq) is added a solution of 4-methylsulfanyl-phenylamine (17 mg, 0.12 mmol, 1.2 eq) in THF (1 mL) and the resulting suspension is stirred for 30 min. Then 1-chloro-4-(6-morpholin-4-yl-pyrazin-2-yl)-isoquinoline (33 mg, 0.1 mmol, 1 eq) is added and the reaction mixture is heated to 80° C. for 4 h. CH₂Cl₂ (5 mL) is added and the solution is washed with H₂O and brine. Chromatography (SiO₂, 10-60% EtOAc-hexanes gradient elution) provided the title compound (33 mg, 99%). MS/ESI, M+1=430.17,

¹H NMR (400 MHz, CDCl₃) 8.35 (d, J=12.0 Hz, 1H), 8.29 (s, 1H), 8.20 (s, 1H), 8.13 (s, 1H), 8.00 (d, J=8.0 Hz, 1H), 7.98 (s, 1H), 7.90 (d, J=8.0 Hz, 1H), 7.69 (t, J=8.0 Hz 2H), 7.65 (d, J=8.0 Hz, 2H), 7.61 (t, J=8.0 Hz 1H), 7.34 (d, J=8.0 Hz, 2H), 3.86 (t, J=4.0 Hz 4H), 3.65 (t, J=8.0 Hz, 4H), 2.50 (s, 3H).

### 19a) 4-(6-bromo-pyrazin-2-yl)-morpholine

PBr₃ (11 g, 36.9 mmol, 5.5 eq) is added to 2,6-dichloro-pyrazine (1.0 g, 6.7 mmol, 1 eq) at rt and heated to 150° C. for 24 h. This solution is dried in vacuum and the residue is dissolved in CH₂Cl₂ (50 mL). The organics are washed with H₂O, brine and dried. Morpholine is added to this solution dropwise at 0° C. and warmed to rt in 5 h. The solution is washed with H₂O and brine. Chromatography (SiO₂, 10-60% EtOAc-hexanes gradient elution) provides product 4-(6-bromo-pyrazin-2-yl)-morpholine (0.5 g, 31%). MS/ESI, M+1=246.01,

¹H NMR (400 MHz, CDCl₃) δ 7.99 (s, 1H), 7.95 (s, 1H), 3.82 (t, J=4.0 Hz, 4H), 3.58 (t, J=4.0 Hz, 4H).

### 19b) 1-Chloro-4-(6-morpholin-4-yl-pyrazin-2-yl)-isoquinoline

A solution of 4-bromo-1-chloro-isoquinoline (400 mg, 1.65 mmol) in THF (20 mL) is cooled to −72° C. A solution of n-BuLi (2.5 M in hexanes) (0.80 mL, 2 mmol) is added dropwise and the reaction temperature maintained at −70° C.˜−68° C. for 30 min. ZnBr₂ (408 mg, 1.91 mmol) is dissolved in THF (6 mL) and is transferred to above mixture slowly at −70° C. The solution is stirred 40 min at −70° C., then warmed to room temperature by removing the cooling bath. Pd(PPh₃)₄ (190 mg, 0.164 mmol) in 6 ml THF and 4-(6-bromo-pyrazin-2-yl)-morpholine (400 mg, 1.65 mmol) in THF (4 mL) are added to the reaction mixture dropwise, then the solution is heated to 60° C. for 30 min and then kept at rt overnight. The reaction mixture is diluted with ethyl acetate, washed with sat. NH₄Cl, then brine, and dried over sodium sulfate. The solution is concentrated until white solid came out from solution. The solid is collected by filtration, washed with ether and dried under vacuum. 300 mg of 1-Chloro-4-(6-morpholin-4-yl-pyrazin-2-yl)-isoquinoline is obtained. Yield was 56%. MS/ESI+, M+1=327

### EXAMPLE 20

### (4-tert-Butyl-phenyl)-[8-chloro-4-(2-morpholin-4-yl-pyrimidin-4-yl)-isoquinolin-1-yl]-amine

(4-tert-Butyl-phenyl)-[8-chloro-4-(2-chloro-pyrimidin-4-yl)-isoquinolin-1-yl]-amine (15 mg, 0.03 mmol) is mixed with morpholine (10 mL) and heated at 80° C. for one hour. Solution is conc in vacuo, and purified on a silica column. Yield: 10 mg, 60% yield. MS: 473.

¹H NMR (400 MHz, CDCl₃) δ 8.42, 8.40 (d, 2H), 8.36 (d, 2H), 8.26 (s, 1H), 7.65-7.41 (m, 5H), 3.88-3.78 (m, 8H), 1.34 (s, 9H).

### 20a) 1,8-Dichloro-isoquinoline

To a solution of 8-chloro-isoquinoline (J. Org. Chem. 1977, 42(19), 3208-9.) (11 g, 54 mmol) in CH₂Cl₂ (200 mL) is added MCPBA (25 g, 112 mmol) in several portions. After stirring for 3 hours, ether (400 mL) is added, followed by addition of hexanes (1 L). Solution stirred overnight and conc in vacuo, ether (200 mL) and hexanes (400 mL) is added, stirred overnight. The ppt is filtered, air dried and mixed with 20 g of PCl₅ and toluene (150 mL). The solution is heated to reflux for 3 h, neutralized with NaHCO₃. Extracted the solution with CH₂Cl₂. Organic layer then dried with sodium sulfate and conc in vacuo, yield 8 g (72%) of 1,8-Dichloro-isoquinoline. MS: 198

### 20b) (4-tert-Butyl-Phenyl)-(8-chloroisoquinolin-1-yl)-amine

A solution of 1,8-dichloro-isoquinoline 8 g, 39 mmol) in butanol (8 mL), HCl (4N solution in dioxane, 6 mL) and 4-tert-butyl-phenylamine (6 g, 40 mmol) is heated at 70° C. for 20 min, conc in vacuo, and a NaHCO₃ solution is added. Extracted with EtOAc, organic layer is then conc in vacuo, purified on a silica column. Yield 3.6 g, 30% of (4-tert-Butyl-phenyl)-(8-chloro-isoquinolin-1-yl)-amine. MS: 310

### 20c) (4-Bromo-8-chloro-isoquinolin-1-yl)-(4-tert-butyl-phenyl)-amine

To an ice-cooled solution of (4-tert-butyl-phenyl)-(8-chloro-isoquinolin-1-yl)-amine (3.6 g, 7 mmol) in THF (20 mL) is added Me₃PhNBr₃ (2.88 g, 7.6 mmol) via several portions. The ice bath is then removed and solution warm to rt, after 15 min, NaHCO₃ solution was added. Extracted with EtOAc, then conc in vacuo, yield 2.6 g 89% of (4-bromo-8-chloro-isoquinolin-1-yl)-(4-tert-butyl-phenyl)-amine. MS: 388

### 20d) (4-Boronic acid-8-chloro-isoquinolin-1-yl)-(4-tert-butyl-phenyl)-amine

A solution of (4-bromo-8-chloro-isoquinolin-1-yl)-(4-tert-butyl-phenyl)-amine (1.6 g, 2.88 mmol) in THF (20 mL) is cooled to −78° C., and n-BuLi (1.6M in hexanes, 3.9 mL) is added dropwise. The solution is kept at −78° C. for 1 h, then the cooling bath is removed and the reaction mixture is slowly warm to rt. After stirring at rt for 30 min, water (1 mL) is then added and solution conc in vacuo. HCl (50 mL, 1M) is added to the crude oil and stirred for 4 h. The solution then decanted, ether is added to form a precipitate. The precipitate is then filtered and air dried, yield 611 mg, 60% of (4-boronic acid-8-chloro-isoquinolin-1-yl)-(4-tert-butyl-phenyl)-amine. MS: 354

### 20e) (4-tert-Butyl-phenyl)-[8-chloro-4-(2-chloro-pyrimidin-4-yl)-isoquinolin-1-yl]-amine

A solution of (4-boronic acid-8-chloro-isoquinolin-1-yl)-(4-tert-butyl-phenyl)-amine (100 mg, 0.28 mmol), 2,4-dichloro-pyrimidine (41.4 mg, 0.28 mmol), PdCl₂(PPh₃)₂ in DME (2 mL) and Na₂CO₃ (2 mL, 1M solution) in a sealed tube was heated at 80° C. for one hour, extracted with CH₂Cl₂ and purified on a silica column, yield 31 mg, 26% yield. MS: 422.

¹H NMR (400 MHz, CDCl₃) δ 9.45 (s, 1H), 8.67 (d, 2H), 8.39 (d, 2H), 8.27 (s, 2H), 7.66-7.60 (m, 3H), 1.35 (s, 9H).

### EXAMPLE 21

### (4-tert-Butyl-phenyl)-[6-fluoro-4-(2-morpholin-4-yl-pyrimidin-4-yl)-isoquinolin-1-yl]-amine

A degassed solution of (4-tert-Butyl-phenyl)-[6-fluoro-4-(4,4,5,5-tetramethyl-[1,3,2]dioxaborolan-2-yl)-isoquinolin-1-yl]-amine, 2,4-dichloropyrimidine (117 mg, 0.785 mmol), K₂CO₃ (291 mg, 2.141 mmol) and Pd(PPh₃)₄ in DME (3 mL) is heated at 60° C. overnight. Water is added to the mixture and extracted with Et₂O. The organic layer is filtered through a silica gel pad, the solution is concentrated to an oil. The oil is dissolved in morpholine (1 mL) and heated at 80° C. overnight. The mixture is concentrated and purified by prep. TLC and then prep HPLC (35%-65% CH₃CN/water in 0.1% TFA). The fraction is free based by sat. NaHCO₃ and extracted with EtOAc to afford brown solid (6 mg). M+H⁺=458.25.

¹H NMR (500 MHz, DMSO) δ 1.31 (s, 9H), 3.70 (m, 4H), 3.77 (m, 4H), 7.02 (d, 1H, J=5.14 Hz), 7.38 (d, 2H, J=8.44 Hz), 7.59 (m, 1H), 7.72 (d, 2H, J=8.80 Hz), 8.30 (m, 2H), 8.47 (d, 1H, J=5.14 Hz), 8.72 (dd, 1H, J=5.87, 9.17 Hz), 9.51 (s, 1H).

### 21a) 1-Chloro-6-fluoro-isoquinoline

A solution of 6-fluoro-2H-isoquinolin-1-one (PCT/GB02/00514; WO 02/062816) (1.3 g, 7.97 mmol) and POCl₃ (3.7 g, 23.9 mmol) In CH₃CN (20 mL) and 4N HCl/dioxane (2 mL) is heated at 50° C. overnight. The reaction mixture is diluted with a NaHCO₃ solution and extracted with EtOAc. The organic layer is concentrated to afford an orange solid (1.1 g, 78%). M+H⁺=181.8.

¹H NMR (400 MHz, CDCl₃) δ 7.42 (m, 2H), 8.26 (m, 3H).

### 21b) (4-tert-Butyl-phenyl)-(6-fluoro-isoquinolin-1-yl)-amine

A solution of 1-chloro-6-fluoro-isoquinoline (1 g, 6.13 mmol) and 4-tert-butyl-aniline (1.1 g, 6.74 mmol) in nBuOH (20 mL) and 4N HCl/dioxane (1 mL) is heated at 80° C. overnight. The mixture is concentrated and the residue is made basic with sat. NaHCO₃ and extracted with EtOAc. The organic layer is dried, concentrated and purified by silica gel column (Hexane to 10% EtOAc/Hexane) to afford yellow solid (900 mg, 56%). M+H⁺=295.3.

¹H NMR (400 MHz, DMSO-d₆) δ 1.29 (s, 9H), 7.13 (d, 1H, J=6 Hz), 7.34 (d, 2H, J=8.67 Hz), 7.50 (m, 1H), 7.60 (dd, 1H, J=2.64, 9.8 Hz) 7.72 (d, 2H, J=8.67 Hz), 7.96 (d, 1H, J=5.65 Hz), 8.61 (dd, 1H J=5.46, 9.23 Hz), 9.16 (s, 1H).

### 21c) (4-Bromo-6-fluoro-isoquinolin-1-yl)-(4-tert-butyl-phenyl)-amine

A solution of (4-tert-Butyl-phenyl)-(6-fluoro-isoquinolin-1-yl)-amine (2.17 g, 7.37 mmol) and PhMe₃NBr₃ (2.93 g, 7.81 mmol) in THF (30 mL) is stirred at 0° C. for 30 min. The THF is evaporated and the solid is dissolved in CH₂Cl₂ and water (200 mL each). The organic layer is washed by water (2×50 mL) and brine (50 mL), dried with Na₂SO₄ and concentrated to afford a light brown solid (2.75 g, 99%). M+H⁺=375.2.

¹H NMR (300 MHz, DMSO) δ 1.29 (s, 9H), 7.36 (d, 2H, J=8.67 Hz), 7.65 (dd, 4H, J=7.35, 8.85 Hz), 8.17 (s, 1H), 8.70 (dd, 1H, J=5.27, 9.42 Hz), 9.38 (s, 1H).

### 21d) (4-tert-Butyl-phenyl)-[6-fluoro-4-(4,4,5,5-tetramethyl-[1,3,2]dioxaborolan-2-yl)-isoquinolin-1-yl]-amine

A degassed solution of (4-bromo-6-fluoro-isoquinolin-1-yl)-(4-tert-butyl-phenyl)-amine (500 mg, 1.34 mmol), bis(pinocolato)diboron (748 mg, 2.93 mmol), KOAc (391 mg, 4.019 mmol) and Pd (pddf) Cl₂, in DMF (10 mL) is heated at 80° C. overnight. Water is added to the mixture and extracted by ether. The ether layer is filtered through a silica gel pad and rotary evaporated down to a brown solid. M+H⁺=421.3. The solid was used in next step without further purification.

### EXAMPLE 22

### (4-tert-Butyl-phenyl)-[6-chloro-4-(2-chloro-pyrimidin-4-yl)-isoquinolin-1-yl]-amine

Prepared by a sequence analogous to Example 21.

MS: 422 ¹H NMR (400 MHz, CDCl₃) δ 8.66 (d, 1H), 8.59 (s, 1H), 8.38 (d, 1H), 8.25 (s, 1H), 8.72 (d, 1H), 7.6-7.5 (m, 5H), 1.34 (s, 9H).

### EXAMPLE 23

### (4-tert-Butyl-phenyl)-[6-chloro-4-(2-morpholin-4-yl-pyrimidin-4-yl)-isoquinolin-1-yl]-amine

Prepared by a sequence analogous to Example 21

MS: 473 ¹H NMR (400 MHz, CDCl₃) δ 8.65 (m, 1H), 8.37-8.32 (m, 2H), 8.15 (s, 1H), 7.55-7.51 (m, 3H), 7.39 (d, 2H), 6.86 (d, 2H).

### EXAMPLE 24

Cyanuric chloride (1.8 g, 10 mmol) and DME (20 mL) were cooled to 0° C., starting amine (3.3 mmol) was added slowly. The ice bath was then removed and solution warmed to rt and stirred overnight. The solution was then conc in vacuo, the solid was mixed with (4-boronic acid-isoquinolin-1-yl)-(4-tert-butyl-phenyl)-amine (3.3 mmol), PdCl₂(PPh₃)₂ (144 mg), DME (6 mL) and Na₂CO₃ (1M solution, 5.4 mL) and heated at 80° C. for two hours. The organic layer separated, conc in vacuo and purified on a reverse phase HPLC system. Isolated yield 5%.

### (4-tert-Butyl-phenyl)-[4-(4-chloro-6-morpholin-4-yl-[1,3,5]triazin-2-yl)-isoquinolin-1-yl]-amine

MS: 475 ¹H NMR (400 MHz, CDCl₃) δ 9.11 (d, 1H), 8.55, (s, 1H), 8.00 (d, 1H), 7.81-7.76 (m, 1H), 7.41-7.36 (m, 1H), 7.32 (d, 2H), 7.19 (d, 2H), 3.94-3.73 (m, 8H), 1.25 (s, 9H).

### (4-tert-Butyl-phenyl)-{4-[4-chloro-6-(2,6-dimethyl-morpholin-4-yl)-[1,3,5]triazin-2-yl]-isoquinolin-1-yl}-amine

MS: 503 ¹H NMR (300 MHz, CDCl₃) δ 9.02 (d, 1H), 8.37 (s, 1H), 8.21 (d, 1H), 7.81-7.76 (m, 1H), 7.49-7.44 (m, 1H), 7.29 (d, 2H), 7.13 (d, 2H), 4.60-4.56 (m, 2H), 3.59-3.54 (m, 2H), 2.27-2.62 (m, 2H), 1.23-1.20 (m, 15H).

### 4-{4-[1-(4-tert-Butyl-phenylamino)-isoquinolin-4-yl]-6-chloro-[1,3,5]triazin-2-yl}-piperazine-1-carboxylic acid ethyl ester

MS: 546 ¹H NMR (300 MHz, CDCl₃) δ 9.08 (d, 1H), 8.57 (s, 1H) 7.97 (d, 1H), 7.79-7.74 (m, 1H), 7.61-7.59 (m, 1H), 7.33 (d, 2H), 7.20 (d, 2H), 4.17-4.10 (q, 2H), 3.93-3.86 (m, 4H), 3.55 (b, 4H), 1.25-1.18 (m, 12H).

### (4-tert-Butyl-phenyl)-[4-(4-chloro-6-thiomorpholin-4-yl-[1,3,5]triazin-2-yl)-isoquinolin-1-yl]-amine

MS: 491 ¹H NMR (300 MHz, CDCl₃) δ 9.07 (d, 1H), 8.49 (s, 1H), 8.06 (d, 1H), 7.80-7.75 (m, 1H), 7.60 (m, 1H), 4.18 (b, 4H), 2.67 (b, 4H), 1.24 (s, 9H).

### EXAMPLE 25

### (4-tert-Butyl-phenyl)-[4-(6-morpholin-4-yl-pyrazin-2-yl)-isoquinolin-1-yl]-amine

Following the general procedure of Suzuki coupling reaction (Example 1). M+H⁺=440.2.

¹H NMR (300 MHz, DMSO-d₆) δ 9.37 (s, 1H), 8.61 (d, J=8.29 Hz, 1H), 8.34 (s, 1H), 8.28 (d, J=7.91 Hz, 1H), 8.16 (d, J=8.67 Hz, 2H), 7.75 (m, 3H), 7.67 (t, J=7.91 Hz, 1H), 7.37 (d, J=8.67 Hz, 2H), 3.75 (m, 4H), 3.60 (m, 4H), 1.31 (s, 9H).

### 25a) 4-(6-Chloro-pyrazin-2-yl)-morpholine

A solution of 2,6-dichloro-pyrazin (2 g, 13.4 mmol) and morpholine (4.7 g, 56.7 mmol) in CH₃CN (50 mL) was stirred overnight. The white solid was filtered off and the solution was concentrated under reduced pressure; The residue was further purified by a short silica gel column to afford product as a white solid (2 g, 75%). M+H⁺=200.13.

¹H NMR (300 MHz, DMSO-d₆) δ 8.29 (s, 1H), 7.9 (s, 1H), 3.70 (m, 4H), 3.54 (m, 4H).

### EXAMPLE 26

### (4-tert-Butyl-phenyl)-[4-(2-morpholin-4-yl-thiazol-4-yl)-naphthalen-1-yl]-amine

Followed the general Suzuki coupling reaction (Example 1). M+H⁺=445.21.

¹H NMR (300 MHz, DMSO-d₆) δ 9.24 (s, 1H), 8.56 (d, J=7.54 Hz, 1H), 8.35 (d, J=8.29 Hz, 1H), 8.12 (s, 1H), 7.75 (m,3H), 7.64 (t, J=7.54 Hz, 1H), 7.35 (d, J=8.67 Hz, 2H), 7.04 (s, 1H), 3.76 (m, 4H), 3.44 (m, 4H), 1.30 (s, 9H)

### 26a) 4-(4-chloro-thiazol-2-yl)-morpholine

A solution of thiazolidine-2,4-dione (0.5 g, 4.27 mmol) and POCl₃ (2 mL, 21 mmol) in CH₃CN (20 mL) and 4N HCl/dioxane (1 mL) was heated to 70° C. overnight. The mixture was poured to the ice water and neutralized with saturated NaHCO₃ then extracted by EtOAc. The organic layer was dried, concentrated and treated with morpholine (1.8 g, 21 mmol). The mixture was stirred at room temperature overnight. The mixture was diluted with EtOAc and washed by water (3×50 mL) and brine (50 mL). The EtOAc phase was concentrated by reduced pressure and purified by silica gel chromatography to afford the product (126 mg, 15%). M+H⁺=205.6.

¹H NMR (300 MHz, DMSO-d₆) δ 6.81 (s, 1H), 3.69 (m, 4H), 3.35 (m, 4H).

### EXAMPLE 27

### (4-tert-Butyl-phenyl)-[4-(2-morpholin-4-yl-1H-imidazol-4-yl)-isoquinolin-1-yl]-amine

A suspension of morpholinoformamidine hydrobromide (40 mg, 0.189 mmol), K₂CO₃ (32 mg, 0.227 mmol), and 2-bromo-1-[1-(4-tert-butyl-phenylamino)-isoquinolin-4-yl]-ethanone (30 mg, 0.076 mmol) in DMF (1 mL) is stirred at rt for 30 minutes, then diluted with water and extracted with Et₂O. The organic phase is dried, concentrated and further purified by preparative TLC, developed by 5% MeOH in EtOAc. Collected the orange color band (Rf=0.394) and extract by EtOAc to afford light yellow compound (20 mg, 60%). M+H⁺=335.1764.

¹H NMR (300 MHz. CD₃OD) δ 8.37 (d, J=7.91 Hz, 1H), 8.12 (d, J=7.91 Hz, 1H), 7.89 (s, 1H), 7.71 (t, J=7.72 Hz, 1H), 7.61 (t, J=7.54 Hz, 1H), 7.52 (d, J=9.04 Hz, 2H), 7.40 (d, J=8.67 Hz, 2H), 6.87 (s, 1H), 3.82 (m, 4H), 3.35 (m, 4H), 1.34 (s, 9H).

### 27a) 1-[1-(4-tert-Butyl-phenylamino)-isoquinolin-4-yl]-2-hydroxy-ethanone

To a solution of (4-bromo-isoquinolin-1-yl)-(4-tert-butyl-phenyl)-amine (500 mg, 1.407 mmol) in anhydrous THF (50 mL) at −78° C. was added a 2.5M solution of BuLi in hexane (1.407 mL, 2.62 mmol). After stirring at the same temperature for 1 h, the mixture was warmed slowly to 40° C. The reaction mixture was cooled to −78° C., then (tert-Butyl-dimethyl-silanyloxy)-acetic acid methyl ester (460 mg, 2.11 mmol) in THF (5 mL) is added slowly. The mixture is stirred at the same temperature for 2 h and then heated to 40° C. for 2 h. The reaction is cooled to rt and quenched by 5 mL of saturated NH₄Cl. The solution is concentrated under vacuum and the mixture is taken by water and EtOAc and extracted with EtOAc and washed with water (20 mL) and brine (20 mL), dried through Na₂SO₄. The solution is concentrated and further purified by a flash) column (100% hexane to 40% EtOAc in hexane) to afford a yellow solid (155 mg, 33%). M+H⁺=335.1764.

¹H NMR (300 MHz, DMSO-d₆) δ 9.76 (s, 1H), 8.99 (d, J=8.67 Hz, 1H), 8.69 (s, 1H), 8.60 (d, J=7.91 Hz, 1H), 7.84 (t, J=7.16 Hz, 1H), 7.72 (d, J=8.67 Hz, 2H), 7.67 (d, J=7.16 Hz, 1H), 7.40 (d, J=8.67 Hz, 2H), 4.96 (t, =5.84 Hz, 1H), 4.75 (d, J=5.28 Hz, 2H), 1.31 (s, 9H).

### 27b) 2-Bromo-1-[1-(4-tert-butyl-phenylamino)-isoquinolin-4-yl]-ethanone

1-[1-(4-tert-Butyl-phenylamino)-isoquinolin-4-yl]-2-hydroxy-ethanone (25 mg, 0.075 mmol) is suspended in CH₂Cl₂, PPh₃ (59 mg, 0.224 mmol) and CBr₄ (74 mg, 0.224 mmol) is added successively. The mixture is stirred overnight. The mixture is loaded to a preparative TLC plate and developed by CH₂Cl₂. The yellow band (Rf=0.5) is collected and extracted with EtOAc to afford a yellow solid (15 mg, 50%). M+H⁺=335.1764.

¹H NMR (300 MHz, DMSO-d₆) δ 9.99 (s, 1H), 9.07 (d, J=8.29 Hz, 1H), 8.98 (s, 1H), 8.73 (d, J=8.29 Hz, 1H), 7.88 (m,3H), 7.72 (t, J=7.16 Hz, 1H), 7.47 (d, J=9.04 Hz, 2H), 4.95 (s, 2H), 1.34 (s, 9H).

### EXAMPLE 28

### (4-tert-Butyl-phenyl)-{4-[2-(tetrahydro-pyran-4-yl)-pyrimidin-4-yl]-isoquinolin-1-yl}-amine

4-chloral Pyrane is added dropwise into THF (5 mL) suspension of Mg (66 mg, 95%, 2.6 mmol) at rt and the solution is heated to reflux for 2 h. After cooled to RT, this solution is transferred to THF solution of compound (4-tert-butyl-phenyl)-[4-(2-chloro-pyrimidin-4-yl)-isoquinolin-1-yl]-amine (50 mg, 013 mmol) at −78° C. Then the solution is warmed to rt in 4 h. CH₂Cl₂ (10 mL) is added and the solution is washed with H₂O and brine. Chromatography (SiO₂, 10-60% EtOAc-hexanes gradient elution) provided product (15 mg, 26%). MS ESI m/z 437 (M+H⁺).

¹H NMR (300 MHz, CDCl₃) δ 8.76 (d, J=6.0 Hz, 1H), 8.51 (d, J=6.0 Hz, 1H), 8.34 (s, 1H), 8.01 (d, J=9.0 Hz, 1H), 7.73 (t, J=9.0 Hz, 1H), 7.65 (m, 3H), 7.43 (m, 3H), 4.15 (m, H), 4.11 (m, 1H), 3.25 (m, 1H), 3.19, 2.38 (s, 3H), 1.26 (d, J=3.0 Hz, 6H).

### EXAMPLE 29

### (4-Isopropyl-phenyl)-[4-(2-morpholin-4-yl-pyrimidin-4-yl)-[2,6]naphthyridin-1-yl]-amine

A degassed solution of (4-boronic acid-[2,6]naphthyridin-1-yl)-(4-isopropyl-phenyl)-amine (400 mg, 1 eq), 4-(4-Bromo-pyrimidin-2-yl)-morpholine (300 mg, 1.2 eq), PdCl₂(PPh₃)₂ (0.1 eq) in DME (5 mL) and Na₂CO₃ (2M, 5 mL) is heated at reflux for 1.5 h. DME is removed in vacuo and residue is dissolved in CH₂Cl₂ (20 mL). After washing with H₂O and brine, this organic solution is dried (MgSO₄) and concentrated to an oil. Chromatography (SiO₂, 40% EtOAc-hexane) provided (4-isopropyl-phenyl)-[4-(2-morpholin-4-yl-pyrimidin-4-yl)-[2,6]naphthyridin-1-yl]-amine (200 mg, 47%) as light yellow solid. HRMS ESI m/z 427.2275 (M+H⁺, C₂₅H₂₇ON₆ requires 427.2246).

¹H NMR (300 MHz, CDCl₃) δ 10.06 (s, 1H), 8.75 (d, J=6.0 Hz, 1H), 8.48 (t, J=3.0 Hz 1H), 7.75 (d, J=6.0 Hz, 1H), 7.64 (d, J=9.0 Hz, 2H), 7.31 (d, J=9.0 Hz, 2H), 6.90 (d, J=3.0 Hz 1H), 3.92 (m, 4H), 3.94 (m, 4H), 2.96 (m, 1H), 1.30 (d, J=6.0 Hz, 6H).

### 29a) (4-Isopropyl-phenyl)-[2,6]naphthyridin-1-yl-amine

A HCl/dioxane solution (4N, 2.18 mL) is added to a solution of 1-chloro-[2,6]naphthyridine (J. Heterocyclic Chem., 18, 1349 (1981) and 4-isopropanylaniline in n-butanol (5 mL) and the resulting solution is heated to 80° C. for 4 h and then evaporated to dryness. The residue is dissolved CH₂Cl₂ (20 mL) and washed with saturated NaHCO₃ (20 mL), H₂O (1×10 mL) and brine (1×10 mL). The organics are dried (Na₂SO₄) and concentrated. Chromatography (SiO₂, 20-80% EtOAc-hexanes gradient elution) provided (4-Isopropyl-phenyl)-[2,6]naphthyridin-1-yl-amine (1.0 g, 48%). MS ESI m/z 264.15 (M+H⁺).

¹H NMR (300 MHz, CDCl₃) δ 9.21 (s, 1H), 8.09 (d, J=6.0 Hz, 1H), 8.25 (d, J=6.0 Hz, 1H), 7.69 (d, J=6.0 Hz, 1H), 7.63 (d, J=9.0 Hz, 2H), 7.28 (d, J=9.0 Hz, 2H), 7.21 (d, J=6.0 Hz, 1H), 7.12 (s, 1H), 2.95 (m, 1H), 1.29 (d, J=6.0 Hz, 6H). ¹³C NMR (75 MHz, CDCl₃) δ 152.4, 144.8, 143.4, 127.4, 121.3, 114.4, 111.1, 77.6, 34.0, 24.5.

### 29b) (4-Bromo-[2,6]naphthyridin-1-yl)-(4-isopropyl-phenyl)-amine

Trimethylphenylammonium tribromide (1.03 g, 2.74 mmol) is added to a solution of (4-Isopropyl-phenyl)-[2,6]naphthyridin-1-yl-amine (680 mg, 2.58 mmol) in THF (10 mL) at 0° C. The solution is warmed up to rt and stirred for 1 h. THF is evaporated to dryness and the residue is dissolved in CH₂Cl₂ (20 mL). The solution is washed with H₂O (1×10 mL) and brine (1×10 mL). The organics are dried (Na₂SO₄) and concentrated to 2 mL. Chromatography (SiO₂, 20-80% EtOAc-hexanes gradient elution) provides (4-Bromo-[2,6]naphthyridin-1-yl)-(4-isopropyl-phenyl)-amine (650 mg, 74%). MS ESI m/z 342 (M+H⁺).

¹H NMR (300 MHz, CDCl₃) δ 9.54 (s, 1H), 8.80 (d, J=6.0 Hz, 1H), 8.34 (s, 1H), 7.67 (d, J=6.0 Hz, 1H), 7.59 (d, J=6.0 Hz, 2H), 7.29 (d, J=9.0 Hz, 2H), 7.14 (s, 1H), 2.95 (m, 1H), 1.29 (d, J=6.0 Hz, 6H).

### 29c) (4-Boronic acid-[2,6]naphthyridin-1-yl)-(4-isopropyl-phenyl)-amine

A solution of BuLi in hexanes (1.1 mL, 2.57 mmol, 2.5 eq) is added to a solution of (4-Bromo-[2,6]naphthyridin-1-yl)-(4-isopropyl-phenyl)-amine (350 mg, 1.eq, 1.02 mmol) in THF (10 mL) at −78° C. The reaction solution is treated with B(O-iPr)₃ (0.31 mL, 1.3 eq) and warmed up to 23° C. in 5 h. The solution is quanched with 0.5 mL of H₂O and dried in in vacuo. The residue is treated with 4N HCl (2 mL) and a light yellow solid precipitates. The solid is filtered and washed with 1N HCl, dried to obtain the crude product (4-boronic acid-[2,6]naphthyridin-1-yl)-(4-isopropyl-phenyl)-amine (400 mg).

### EXAMPLE 30

### 4-[1-(4-tert-Butyl-phenylamino)-isoquinolin-4-yl]-pyrimidine-2-carbonitrile

A solution of (4-tertbutyl-phenyl)-[4-(2-chloro-pyrimidin-4-yl)-isoquinolin-1-yl]-amine (25 mg, 0.064 mmol), KCN (8.4 mg. 2 eq) and PdCl₂(PPh₃)₄ (5 mg) and Et₃N (10 mg) in DMF (1 mL) is heated at 80° C. for 4 h. 10 ml DCM is added and the solution is washed with NH₄Cl (10 ml), H₂O and brine. Chromatography (SiO₂, 10-60% EtOAc-hexanes gradient elution) provides the title compound (24 mg, 99%). MS ESI m/z 380.20 (M+H).

¹H NMR (300 MHz, CDCl₃) δ 8.89 (d, J=6.0 Hz, 1H), 8.48 (t, J=9.0 Hz, 2H), 8.24 (s, 1H), 8.00 (d, J=6.0 Hz, 1H), 7.81 (t, J=6.0 Hz, 1H), 7.70 (d, J=6.0 Hz, 1H), 7.60 (d, J=9.0 Hz, 2H), 7.45 (d, J=9.0 Hz; 2H), 1.36 (s, 9H).

### BIOLOGICAL EXAMPLES

Active B-Raf, C-Raf, and V599E B-Raf proteins of human sequence are purified from insect cells using the baculoviral expression system. Raf inhibition is tested in 96-well microplates coated with IκB-α and blocked with Superblock. The phosphorylation of IκB-α at Serine 36 is detected using a phospho-IκB-α specific antibody (Cell Signaling #9246), an anti-mouse IgG alkaline phosphatase conjugated secondary antibody (Pierce #31320), and an alkaline phosphatase substrate, ATTOPHOS (Promega, #S101).

The following compounds in Tables 2 and 3 inhibit wild-type C-Raf at an IC₅₀ of from 0.05 mmol/L to more than 4.0 mmol/L and/or mutant B-Raf (V599E) at an IC₅₀ of from 0.08 mmol/L to more than 4.0 mmol/L.

TABLE 2

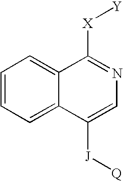

| | J-Q | X-Y | m.p. ° C. | M + 1 |
|---|---|---|---|---|
| 1 | 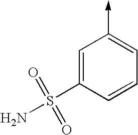 | 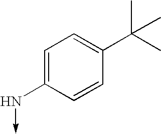 | | 431 |
| 2 | 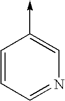 | 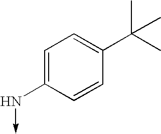 | 164-166 | 354.3 |
| 3 | 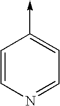 | 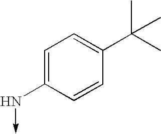 | 182-185 | 354.2 |
| 4 | 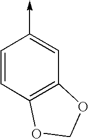 | 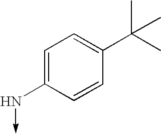 | 154-156 | 397.2 |
| 5 | 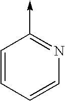 | 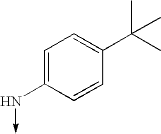 | 183.1-186.6 | 354.2 |
| 6 | 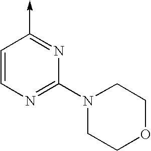 | 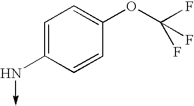 | | 468.2 |
| 7 | 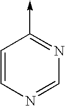 | 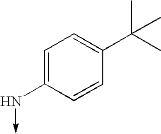 | | 355.2 |
| 8 | 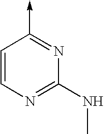 | 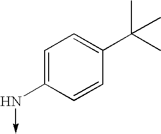 | | 384.2 |
| 9 | 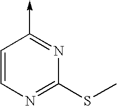 | 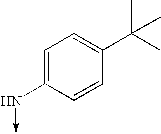 | | 401.2 |
| 10 | 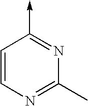 | 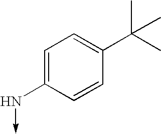 | | 396.2 |
| 11 | 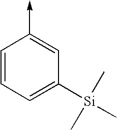 | 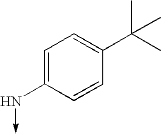 | 95 | 415 |
| 12 | 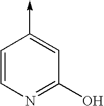 | 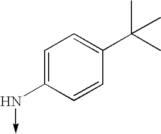 | 202.9-203.4 | 369.3 |
| 13 | 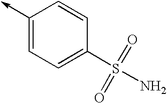 | 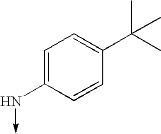 | | 432.2 |
| 14 | 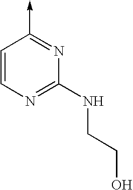 | 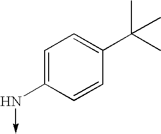 | | 414.2 |
| 15 | 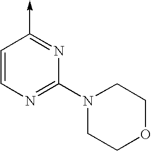 | 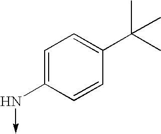 | | 440.3 |
| 16 | 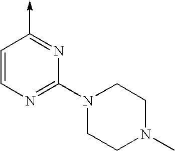 | 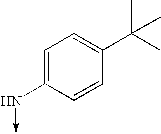 | | 453.3 |
| 17 | 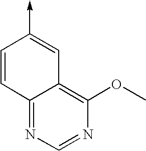 | 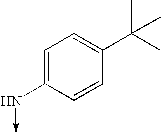 | | 434 |
| 18 | 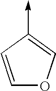 | 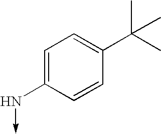 | 183-185 | 343 |
| 19 | 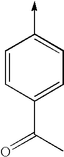 | 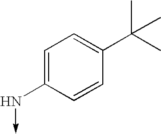 | | 395.3 |
| 20 | 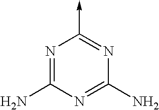 | 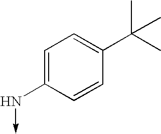 | | 385 |
| 21 | 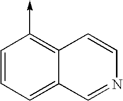 | 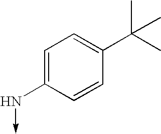 | | 403 |
| 22 | 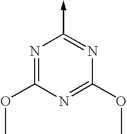 | 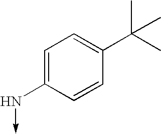 | | 415 |
| 23 | 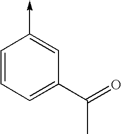 | 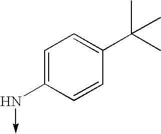 | | 395.3 |
| 24 | 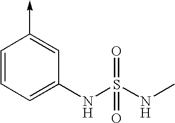 | 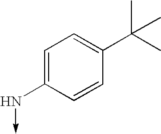 | 114-117 | 461.3 |
| 25 | 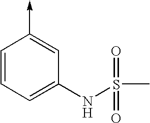 | 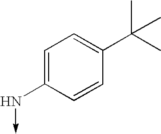 | 217-220 | 446.3 |
| 26 | 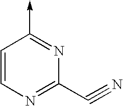 | 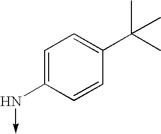 | | 380.2 |
| 27 | 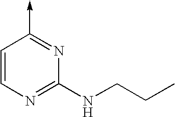 | 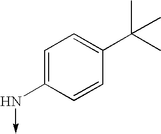 | 150-152 | 412.5 |
| 28 | 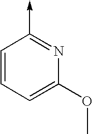 | 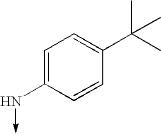 | 102.9 | 384.3 |
| 29 | 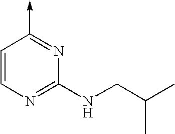 | 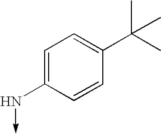 | 94.6-96.2 | 426.3 |
| 30 | 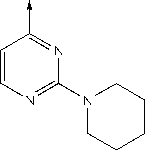 | 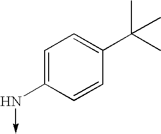 | 91.7-91.8 | 438.4 |
| 31 | 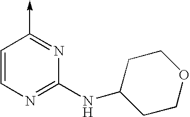 | 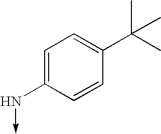 | 136.7-137.9 | 454.3 |
| 32 | 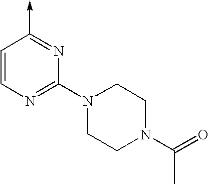 | 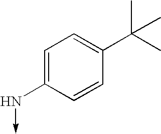 | | 481 |
| 33 | 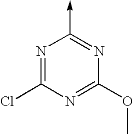 | 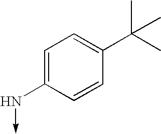 | | 420 |
| 34 | 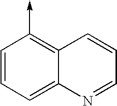 | 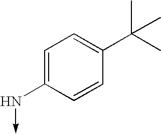 | | 403 |
| 35 | 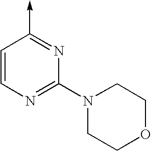 | 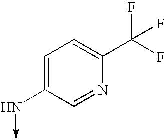 | | 453 |
| 36 | 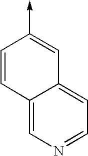 | 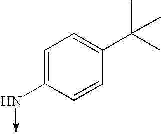 | | 404.3 |
| 37 | 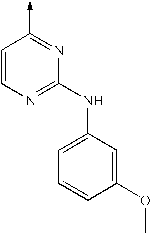 | 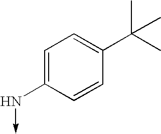 | | 476.4 |
| 38 | 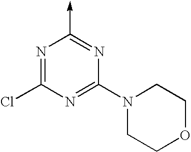 | 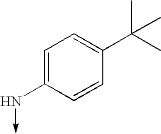 | | 475 |
| 39 | 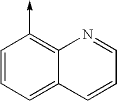 | 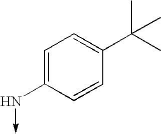 | | 404 |
| 40 | 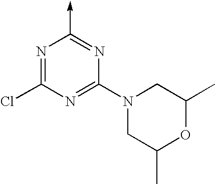 | 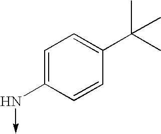 | | 503 |
| 41 | 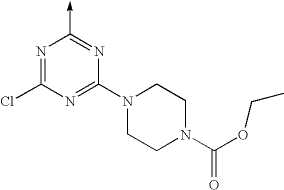 | 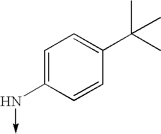 | | 546 |
| 42 | 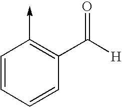 | 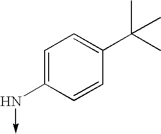 | | 380 |
| 43 | 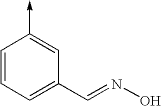 | 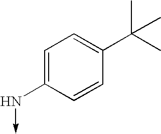 | | 476.4 |
| 44 | 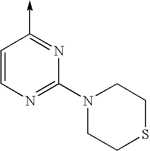 | 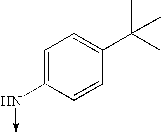 | 123.4-125.8 | 456.3 |
| 45 | 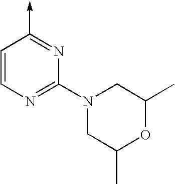 | 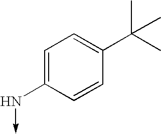 | 144.1-144.2 | 468.3 |
| 46 | 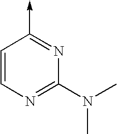 | 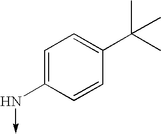 | | 398.3 |
| 47 | 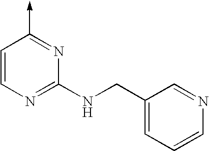 | 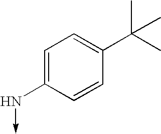 | | 461.4 |
| 48 | 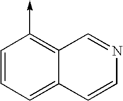 | 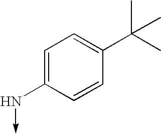 | | 404 |
| 49 | 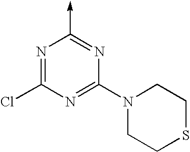 | 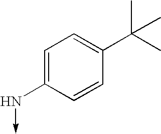 | | 491 |
| 50 | 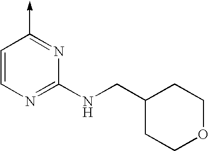 | 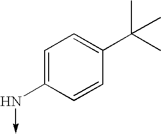 | | 468.3 |
| 51 | 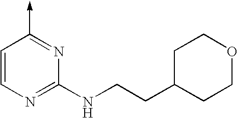 | 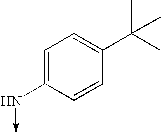 | | 482 |
| 52 | 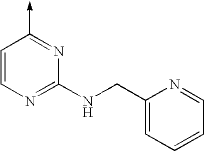 | 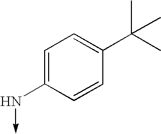 | | 461.3 |
| 53 | 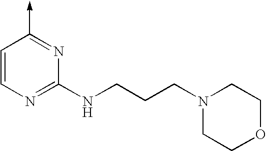 | 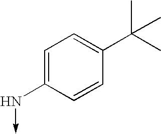 | | 497.4 |
| 54 | 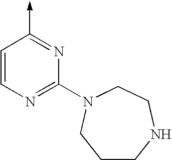 | 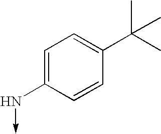 | | 453.4 |
| 55 | 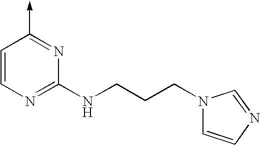 | 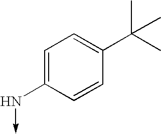 | | 478.3 |
| 56 | 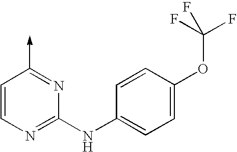 | 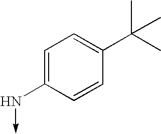 | | 530.2 |
| 57 | 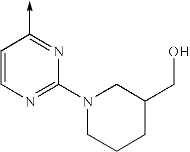 | 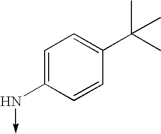 | 106-108 | 482 |
| 58 | 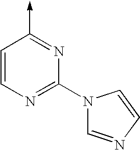 | 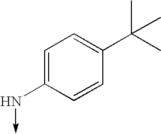 | 234.5-234.6 | 421.3 |
| 59 | 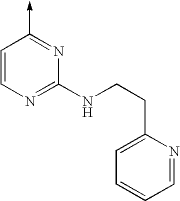 | 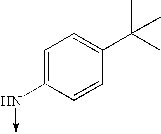 | | 475.5 |
| 60 | 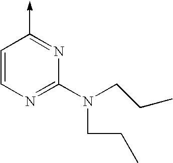 | 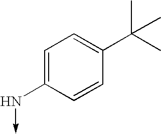 | | |
| 61 | 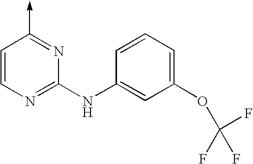 | 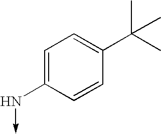 | | 530.3 |
| 62 | 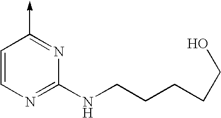 | 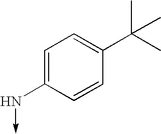 | 68.9-70.2 | 456.3 |
| 63 | 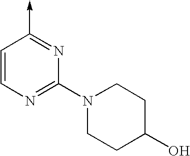 | 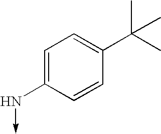 | | 454.4 |
| 64 | 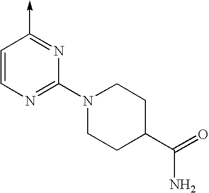 | 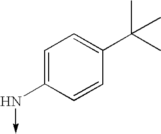 | | 481.4 |
| 65 | 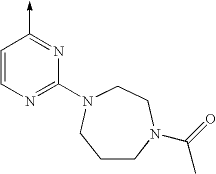 | 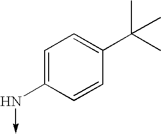 | | 495.4 |
| 66 | 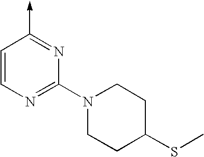 | 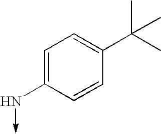 | 138-139 | 484 |
| 67 | 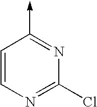 | 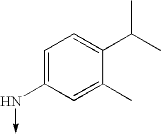 | 120.8-120.9 | 389.2 |
| 68 | 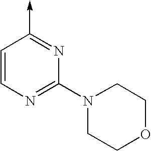 | 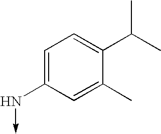 | 208.3-208.4 | 440.3 |
| 69 | 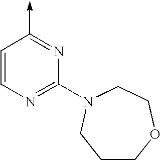 | 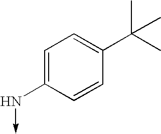 | 95-99 | 454.3 |
| 70 | 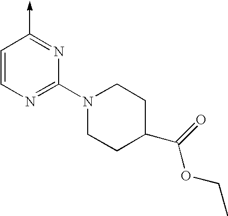 | 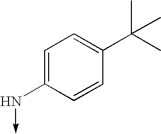 | | 510.3 |
| 71 | 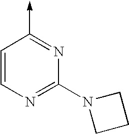 | 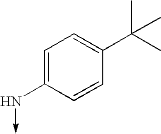 | 171-173 | 410 |
| 72 | 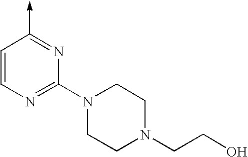 | 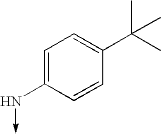 | | 483.5 |
| 73 | 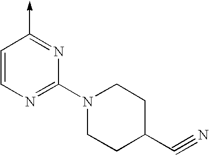 | 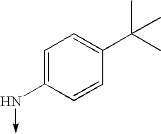 | | 463.4 |
| 74 | 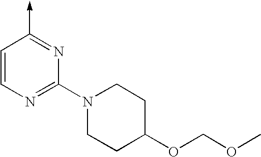 | 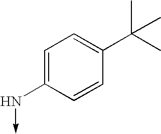 | 143-145 | 498 |
| 75 | 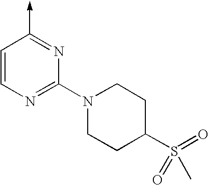 | 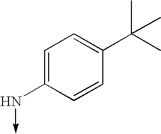 | 135-138 | 516 |
| 76 | 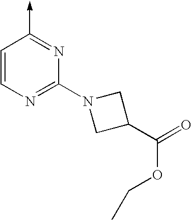 | 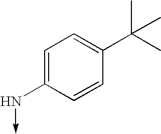 | 181 | 482 |
| 77 | 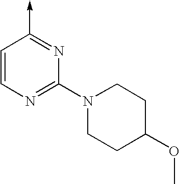 | 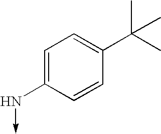 | 138 | 468 |
| 78 | 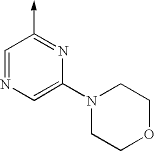 | 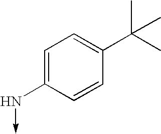 | 143-145 | 440 |
| 79 | 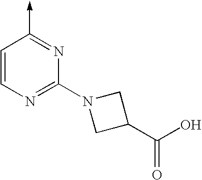 | 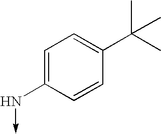 | | 454 |
| 80 | 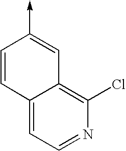 | 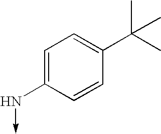 | | 438 |
| 81 | 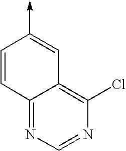 | 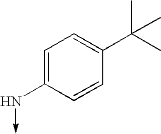 | | 438 |
| 82 | 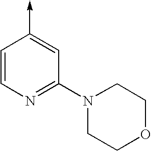 | 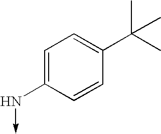 | 207.1-207.4 | 439.2 |
| 83 | 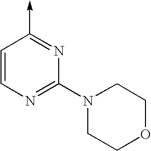 | 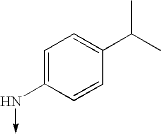 | 180-182.7 | 426.3 |
| 84 | 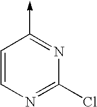 | 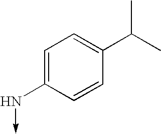 | 100.3-104.4 | 375.2 |
| 85 | 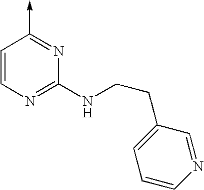 | 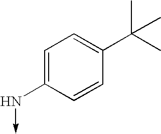 | | 475.5 |
| 86 | 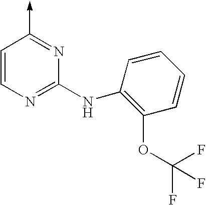 | 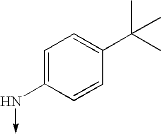 | | 530.4 |
| 87 | 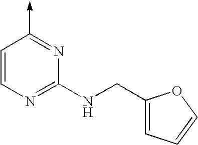 | 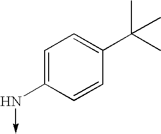 | | 450.3 |
| 88 | 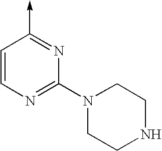 | 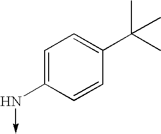 | | 439.3 |
| 89 | 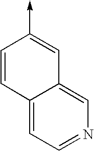 | 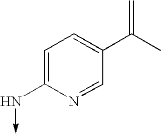 | | 389 |
| 90 | 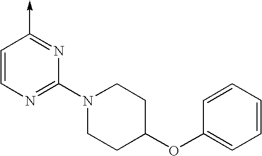 | 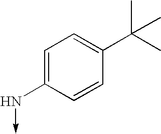 | 166 | 530 |
| 91 | 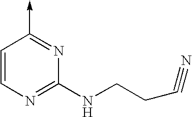 | 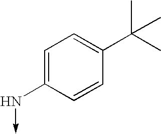 | | 423 |
| 92 | 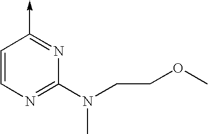 | 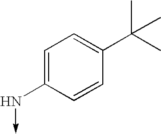 | | 442.4 |
| 93 | 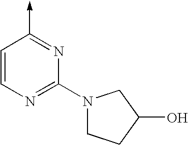 | 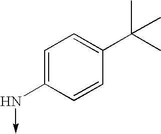 | 135-141 | 440 |
| 94 | 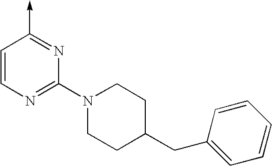 | 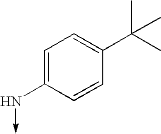 | | 528.1 |
| 95 | 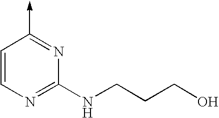 | 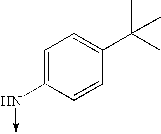 | | 428.4 |
| 96 | 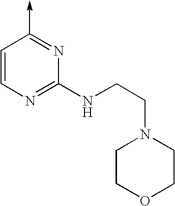 | 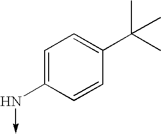 | | 483.5 |
| 97 | 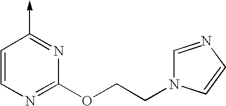 | 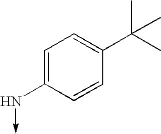 | | 465 |
| 98 | 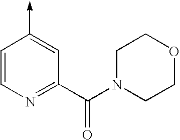 | 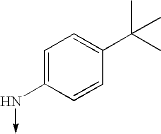 | | |
| 99 | 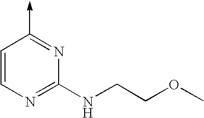 | 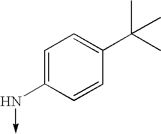 | | 428.4 |
| 100 | 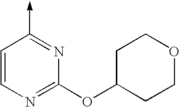 | 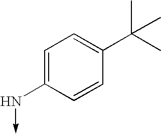 | 120.6-123.3 | 455.2 |
| 101 | 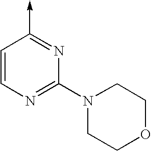 | 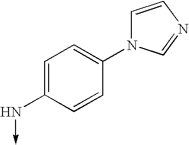 | | 450.2 |
| 102 | 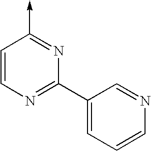 | 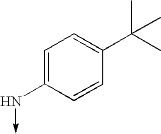 | | 431.3 |
| 103 | 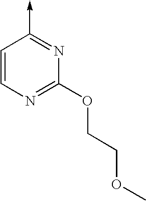 | 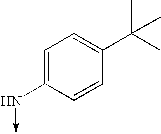 | | 429 |
| 104 | 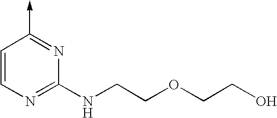 | 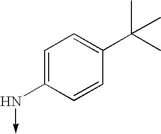 | 138.5-136.6 | 458.2 |
| 105 | 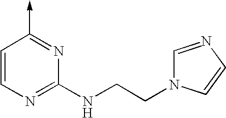 | 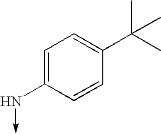 | | 464.3 |
| 106 | 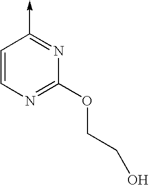 | 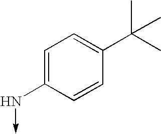 | | 415 |
| 107 | 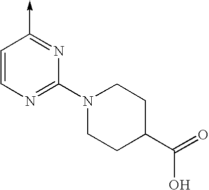 | 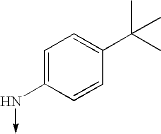 | 206.5-206.6 | 482.4 |
| 108 | 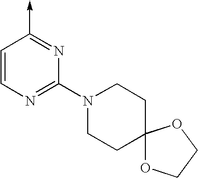 | 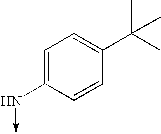 | | 496.4 |
| 109 | 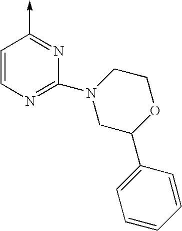 | 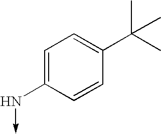 | 177 | 516 |
| 110 | 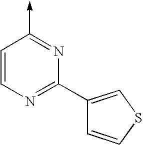 | 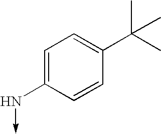 | | 436.2 |
| 111 | 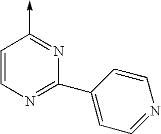 | 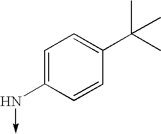 | | 431.4 |
| 112 | 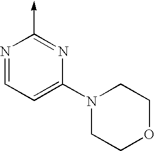 | 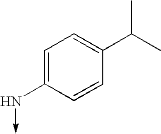 | 156-157 | 426.4 |
| 113 | 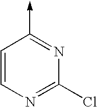 | 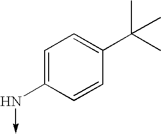 | 257-258 | 389.2 |
| 114 | 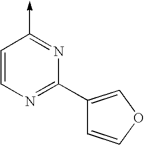 | 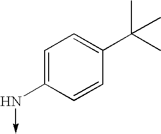 | | 420.2 |
| 115 | 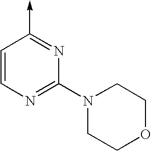 | 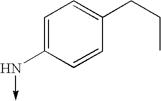 | 165-167 | 426 |
| 116 | 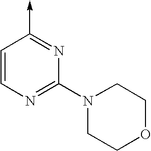 | 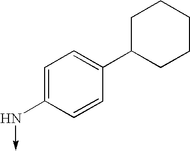 | 111-113 | 466 |
| 117 | 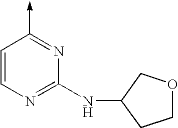 | 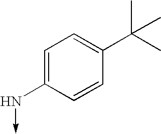 | | 440 |
| 118 | 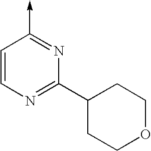 | 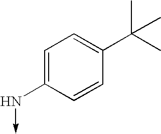 | | 439.8 |
| 119 | 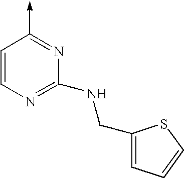 | 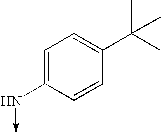 | 155-157 | 466 |
| 120 | 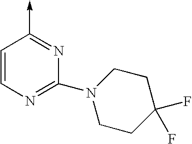 | 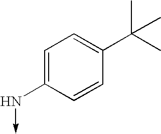 | 93-95 | 474 |
| 121 | 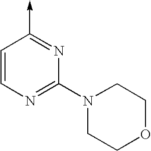 | 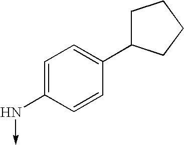 | 150-152 | 452 |
| 122 | 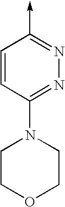 | 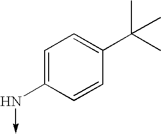 | 226-229 | 440 |
| 123 | 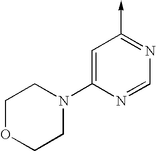 | 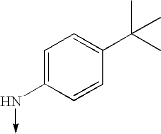 | 147-150 | 440 |
| 124 | 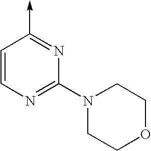 | 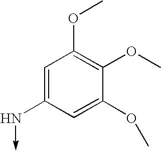 | 142-143 | 473 |
| 125 | 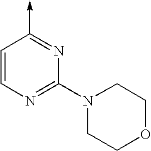 | 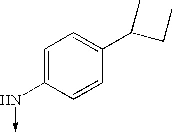 | 149-151 | 440 |
| 126 | 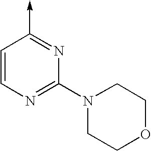 | 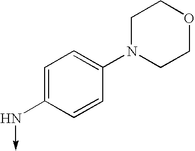 | 235-237 | 469 |
| 127 | 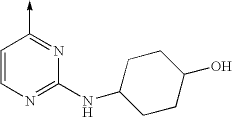 | 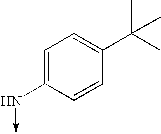 | 171.4-171.5 | 468.2 |
| 128 | 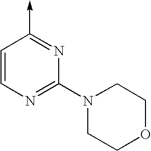 | 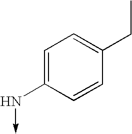 | | 412.2 |
| 129 | 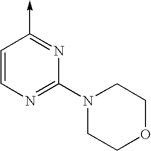 | 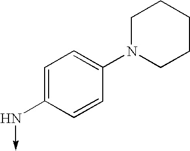 | 124-126 | 467 |
| 130 | 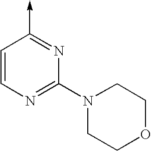 | 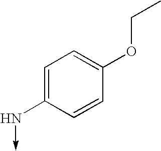 | | 428.1 |
| 131 | 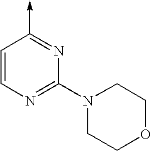 | 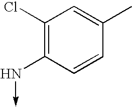 | | 432.6 |
| 132 | 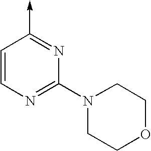 | 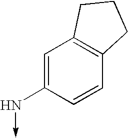 | | 424.2 |
| 133 | 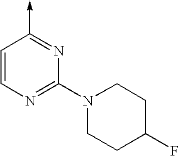 | 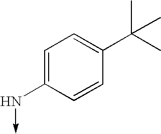 | 100-101 | 456 |
| 134 | 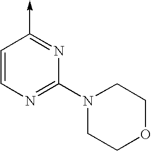 | 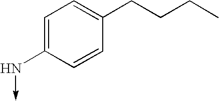 | | 440.2 |
| 135 | 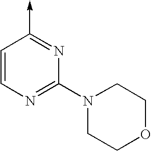 | 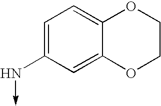 | 106-107 | 442 |
| 136 | 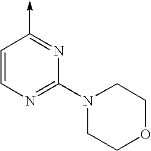 | 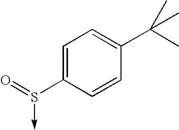 | | 473.2 |
| 137 | 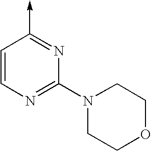 | 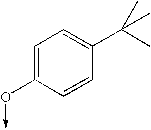 | | 441.2 |
| 138 | 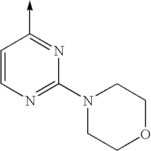 | 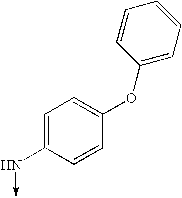 | 119-121 | 476 |
| 139 | 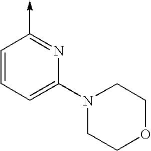 | 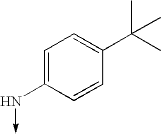 | | 439 |
| 140 | 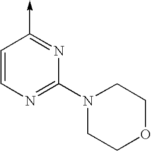 | 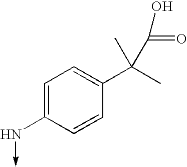 | | 470.4 |
| 141 | 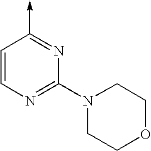 | 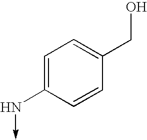 | | 428.2 |
| 142 | 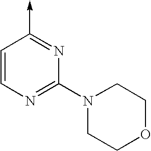 | 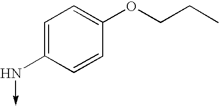 | 168-169 | 442 |
| 143 | 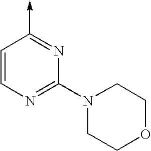 | 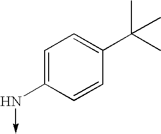 | 157-158 | 446 |
| 144 | 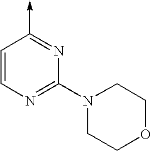 | 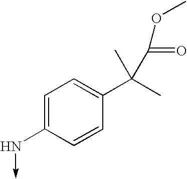 | | 484.3 |
| 145 | 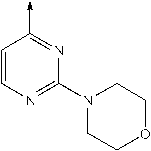 | 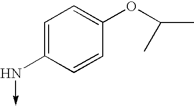 | 211.7-212.6 | 442.3 |
| 146 | 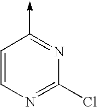 | 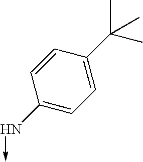 | | 389.15 |
| 147 | 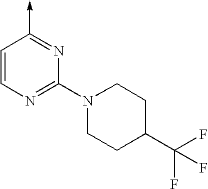 | 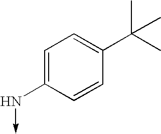 | 179-180 | 506 |
| 148 | 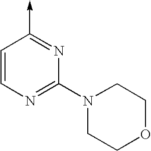 | 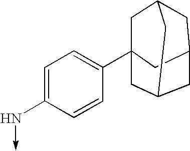 | | 518.3 |
| 149 | 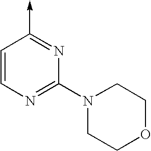 | 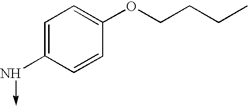 | 158-159 | 456 |
| 150 | 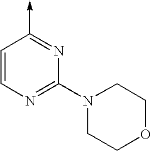 | 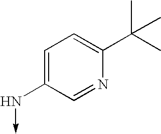 | | 441.5 |
| 151 | 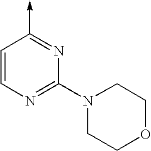 | 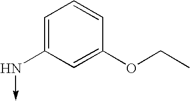 | | 428 |
| 152 | 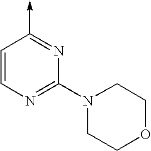 | 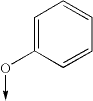 | | 385.4 |
| 153 | 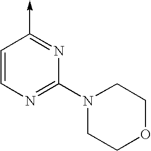 | 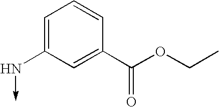 | 85-87 | 456 |
| 154 | 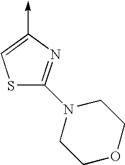 | 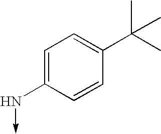 | | |
| 155 | 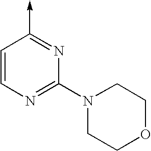 | 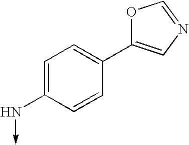 | | 451.2 |
| 156 | 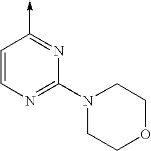 | 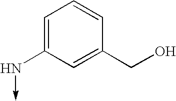 | 158-159 | 414 |
| 157 | 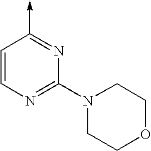 | 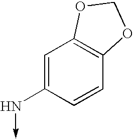 | 132-138 | 428.1 |
| 158 | 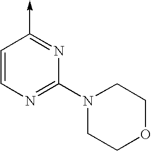 | 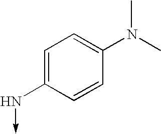 | 134.8-141.9 | 427.4 |
| 159 | 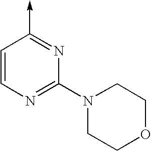 | 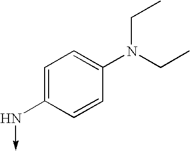 | 145-146 | 455.48 |
| 160 | 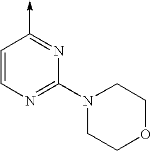 | 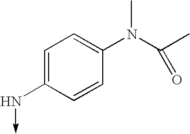 | 195-196 | 455 |
| 161 | 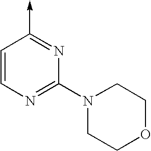 | 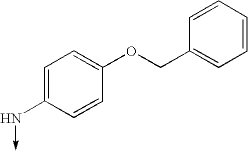 | 69-73 | 490 |
| 162 | 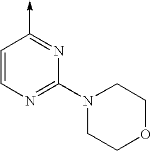 | 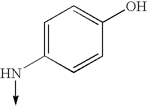 | | 400 |
| 163 | 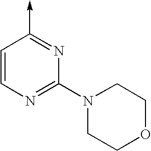 | 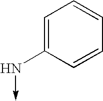 | | 384.3 |
| 164 | 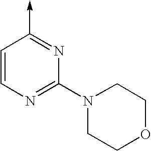 | 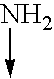 | 234 | 308.3 |
| 165 | 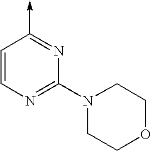 | 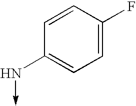 | 115-117 | 402 |
| 166 | 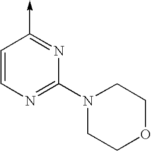 | 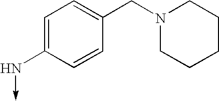 | | 481 |
| 167 | 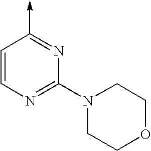 | 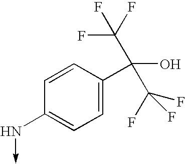 | | 550 |
| 168 | 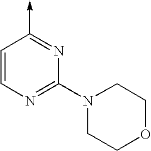 | 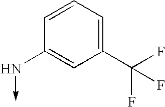 | | 452 |
| 169 | 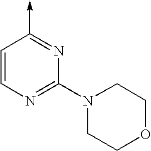 | 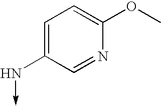 | 183-184 | 415 |
| 170 | 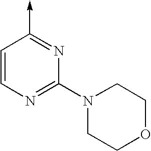 | 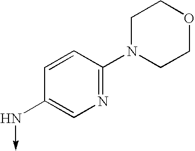 | 213-214 | 470 |
| 171 | 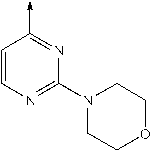 | 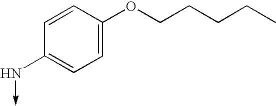 | | 470 |
| 172 | 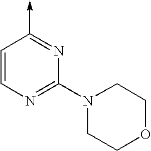 | 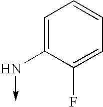 | | 401 |
| 173 | 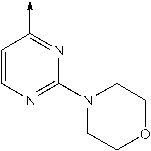 | 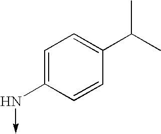 | | 426 |
| 174 | 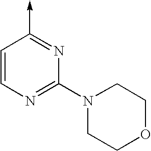 | 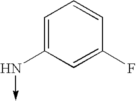 | | 402 |
| 175 | 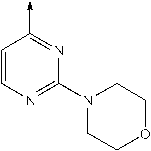 | 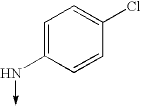 | 125-127 | 418 |
| 176 | 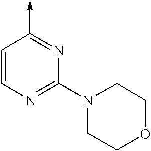 | 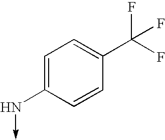 | | 451 |
| 177 | 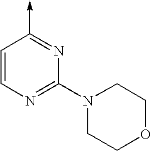 | 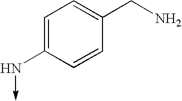 | | 412 |
| 178 | 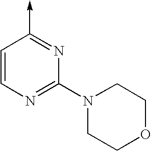 | 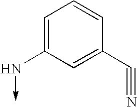 | | 408 |
| 179 | 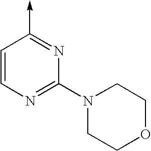 | 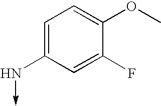 | | 431 |
| 180 | 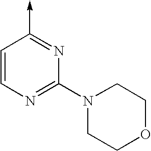 | 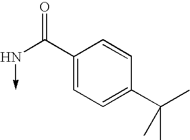 | | 468 |
| 181 | 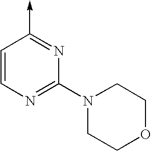 | 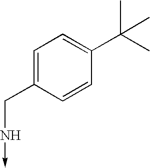 | | 454 |
| 182 | 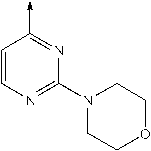 | 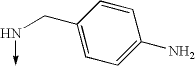 | | 412 |
| 183 | 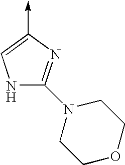 | 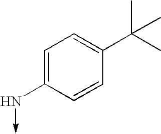 | | 445 |
| 184 | 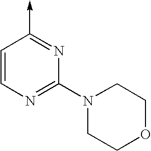 | 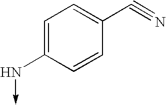 | | 408 |
| 185 | 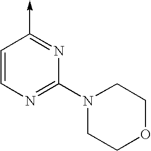 | 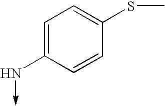 | | 429 |
| 186 | 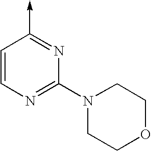 | 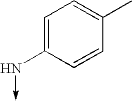 | | 397 |
| 187 | 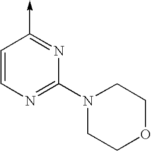 | 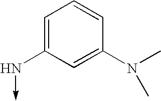 | | 426 |
| 188 | 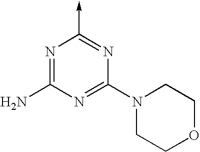 | 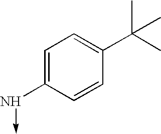 | | |
| 189 | 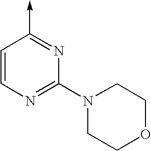 | 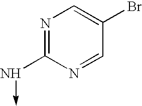 | | 464 |
| 190 | 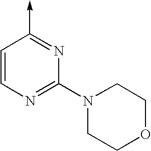 | 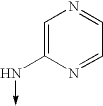 | | 386 |
| 191 | 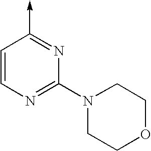 | 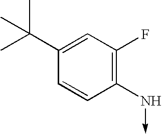 | | 458 |
| 192 | 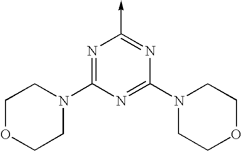 | 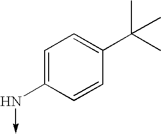 | | 526 |
| 193 | 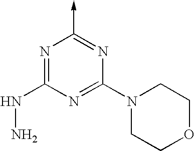 | 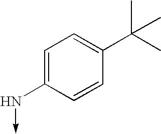 | | |
| 194 | 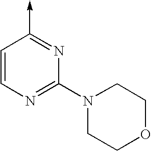 | 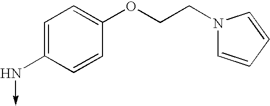 | 218-219 | 494 |
| 195 | 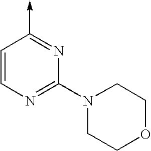 | 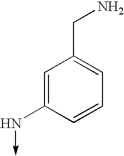 | | 412 |
| 196 | 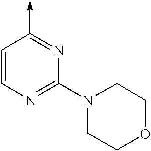 | 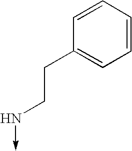 | | 412 |
| 197 | 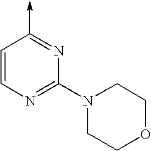 | 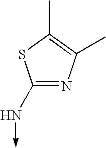 | | 419 |
| 198 | 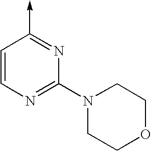 | 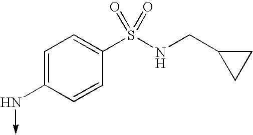 | | 517 |
| 199 | 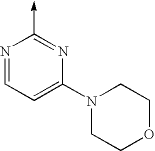 | 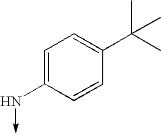 | | 440 |
| 200 | 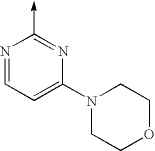 | 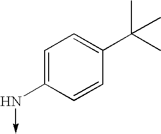 | | 439 |
| 201 | 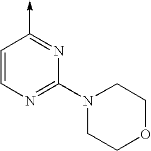 | 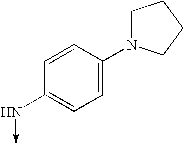 | | 452 |
| 202 | 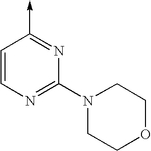 | 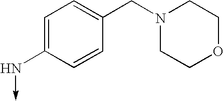 | | 482 |
| 203 | 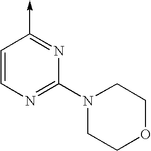 | 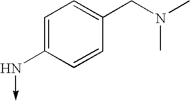 | | 440 |
| 204 | 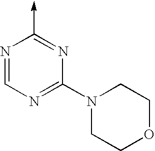 | 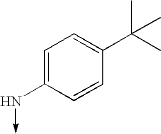 | | 441 |
| 205 | 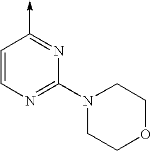 | 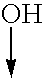 | 127-128 | 308 |
| 206 | 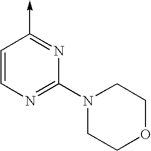 | 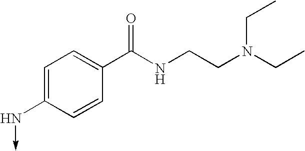 | 65-71 | 525 |
| 207 | 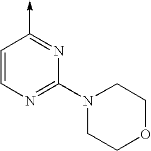 | 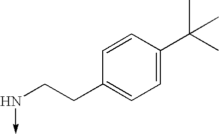 | | 468 |
| 208 | 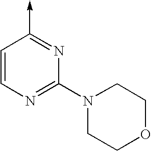 | 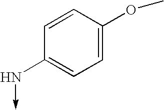 | 160-161 | 414 |
| 209 | 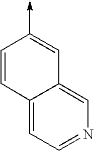 | 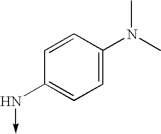 | | 391 |
| 210 | 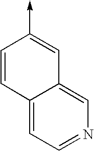 | 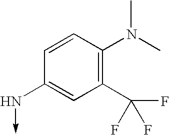 | | 459 |
| 211 | 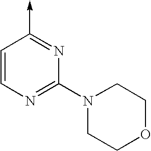 | 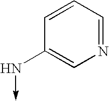 | | 385 |
| 212 | 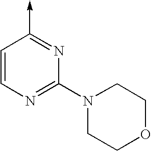 | 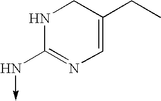 | | |
| 213 | 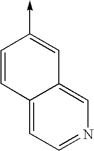 | 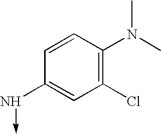 | | 425 |
| 214 | 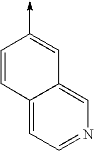 | 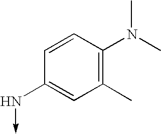 | | 405 |
| 215 | 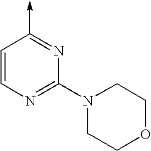 | 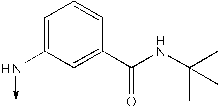 | | 483 |
| 216 | 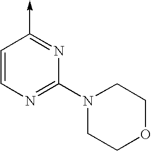 | 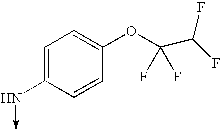 | | 500 |
| 217 | 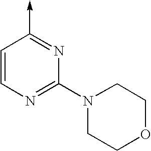 | 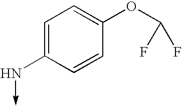 | | 450 |
| 218 | 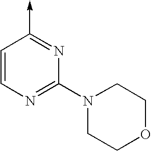 | 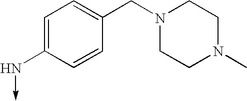 | | 496 |
| 219 | 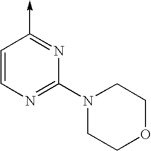 | 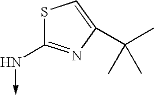 | | 447 |
| 220 | 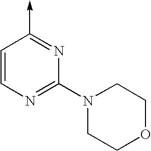 | 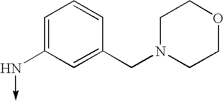 | | 483 |
| 221 | 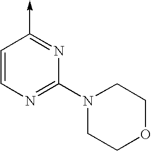 | 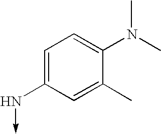 | | 441 |
| 222 | 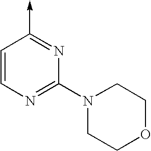 | 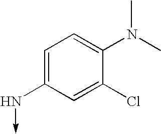 | | 461 |
| 223 | 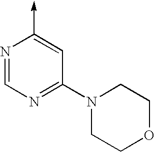 | 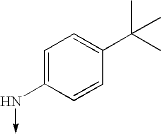 | | 426 |
| 224 | 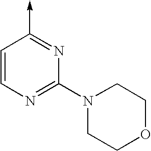 | 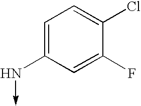 | 147-148 | 435 |
| 225 | 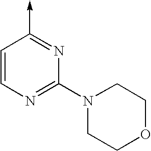 | 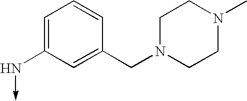 | | 496 |
| 226 | 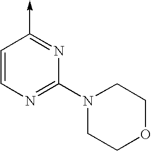 | 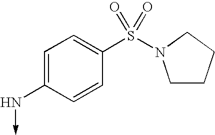 | FOAM | 516 |
| 227 | 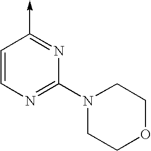 | 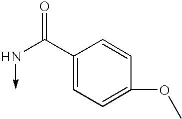 | | 442 |
| 228 | 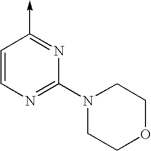 | 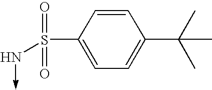 | | 504 |
| 229 | 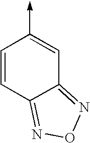 | 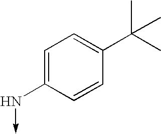 | | 394 |
| 230 | 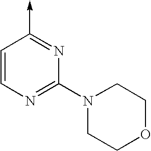 | 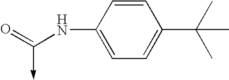 | | 467 |
| 231 | 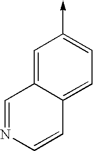 | 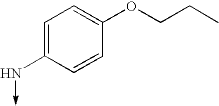 | | 405 |
| 232 | 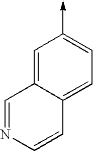 | 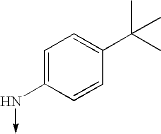 | 217-219 | 404 |
| 233 | 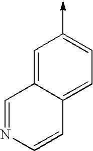 | 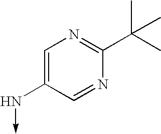 | 231-232 | 406 |
| 234 | 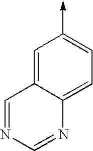 | 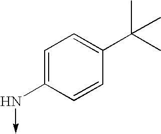 | 213-214 | 405 |
| 235 | 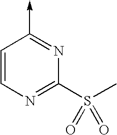 | 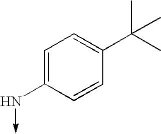 | | 433 |
| 236 | 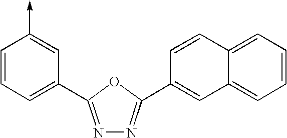 | 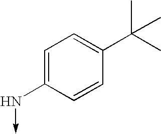 | | 547 |
| 237 | 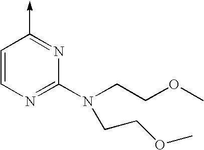 | 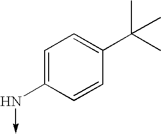 | 84-89 | 486 |
| 238 | 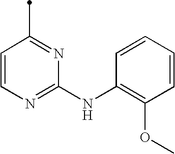 | 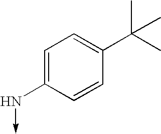 | 99.5-99.6 | 476 |
| 239 | 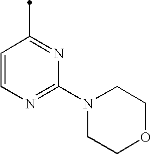 | 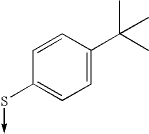 | | 457 |
| 240 | 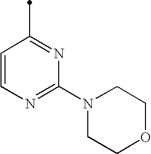 | 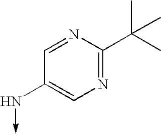 | | 442 |
| 241 | 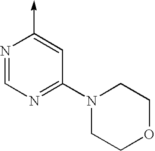 | 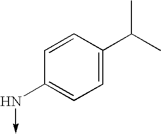 | 197.5-197.6 | 426 |
| 242 | 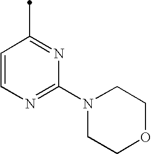 | 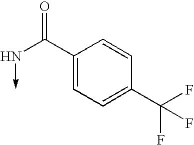 | | 480 |
| 243 | 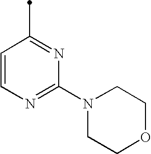 | 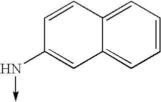 | | 434 |
| 244 | 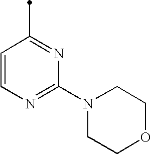 | 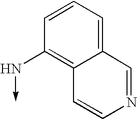 | | 435 |
| 245 | 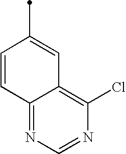 | 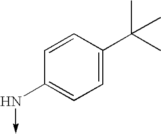 | | 439 |
| 246 | 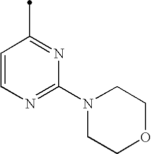 | 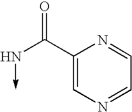 | | 414 |
| 247 | 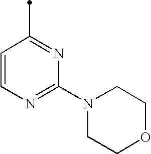 | 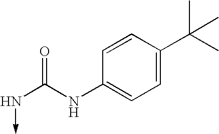 | | 483 |
| 248 | 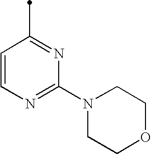 | 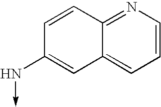 | | 435 |
| 249 | 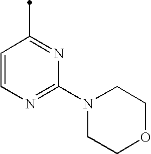 | 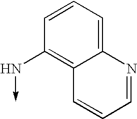 | | 435 |
| 250 | 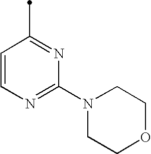 | 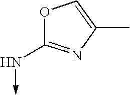 | | 389 |
| 251 | 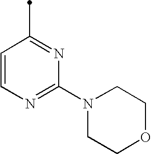 | 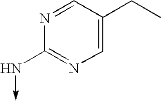 | | 414 |
| 252 | 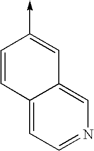 | 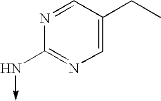 | | 378 |
| 253 | 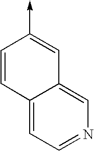 | 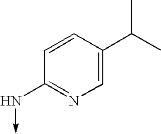 | | 391 |
| 254 | 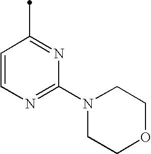 | 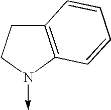 | | 410 |
| 255 | 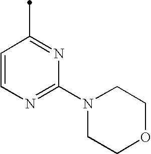 | 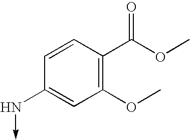 | 150-151 | 472 |
| 256 | 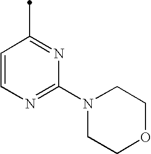 | 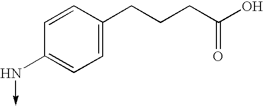 | 278-279 | 470 |
| 257 | 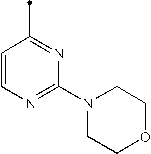 | 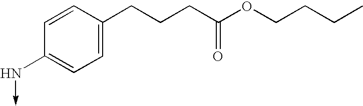 | 140-141 | 512 |
| 258 | 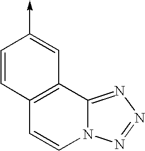 | 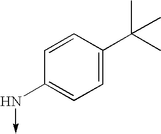 | | 445 |
| 259 | 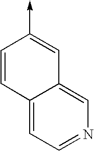 | 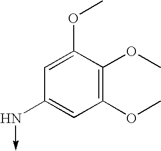 | | 438 |
| 260 | 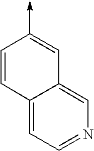 | 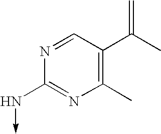 | | 404 |
| 261 | 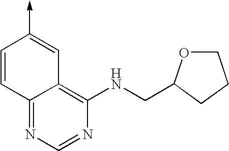 | 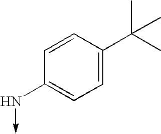 | | 504 |
| 262 | 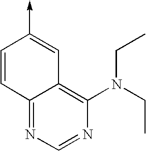 | 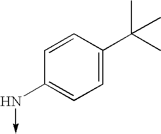 | | 476 |
| 263 | 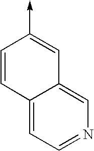 | 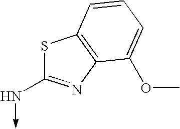 | | 435 |
| 264 | 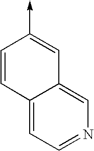 | 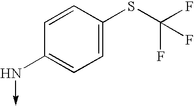 | | 448 |
| 265 | 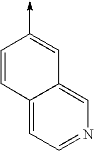 | 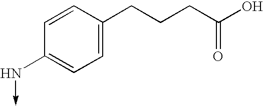 | | 434 |
| 266 | 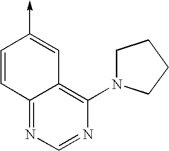 | 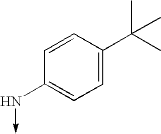 | | 474 |
| 267 | 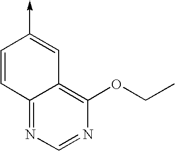 | 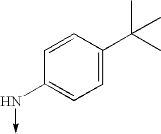 | | 449 |
| 268 | 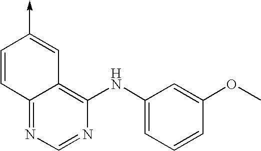 | 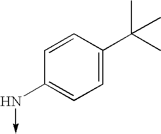 | | 526 |
| 269 | 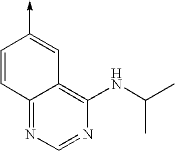 | 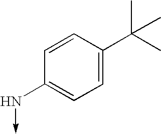 | | 462 |
| 270 | 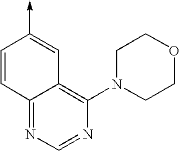 | 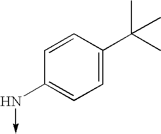 | | 490 |
| 271 | 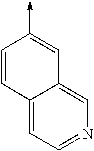 | 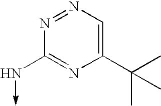 | | 406 |
| 272 | 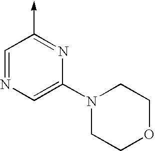 | 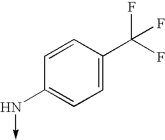 | | 452 |
| 273 | 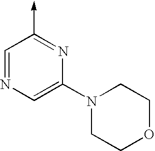 | 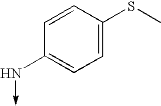 | | 430 |
| 274 | 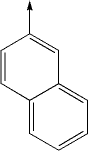 | 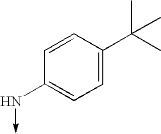 | | 403 |
| 275 | 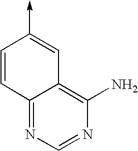 | 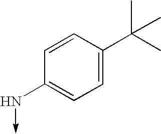 | | 420 |
| 276 | 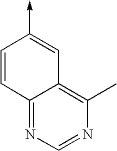 | 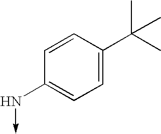 | | 419 |
| 277 | 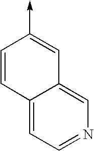 | 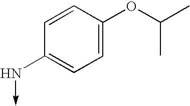 | | 406 |
| 278 | 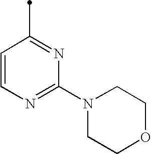 | 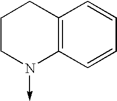 | 101-104 | 424 |
| 279 | 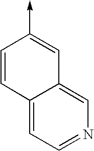 | 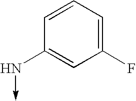 | | 366 |
| 280 | 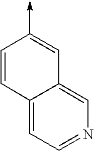 | 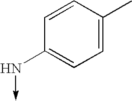 | | 362 |
| 281 | 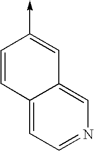 | 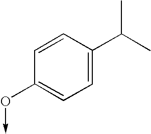 | | 391 |
| 282 | 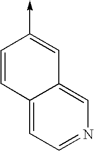 | 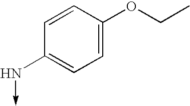 | | 392 |
| 283 | 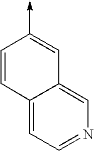 | 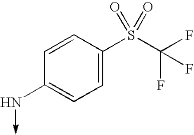 | | 480 |
| 284 | 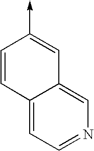 | 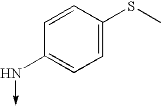 | 168-173 | 394 |
| 285 | 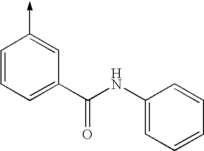 | 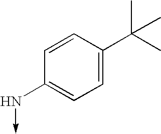 | | 472 |
| 286 | 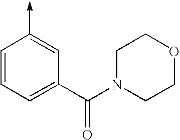 | 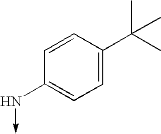 | | 466 |
| 287 | 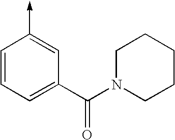 | 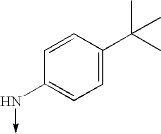 | | 465 |
| 288 | 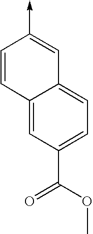 | 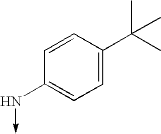 | | 461 |
| 289 | 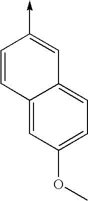 | 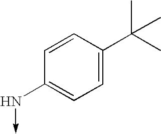 | | 433 |
| 290 | 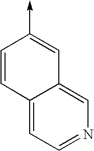 | 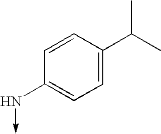 | | 390 |
| 291 | 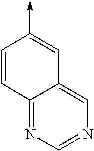 | 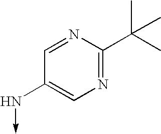 | | 407 |
| 292 | 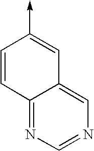 | 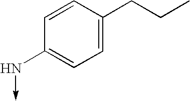 | | 391 |
| 293 | 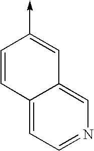 | 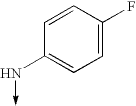 | | 366 |
| 294 | 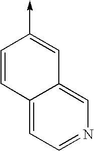 | 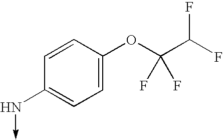 | | 464 |
| 295 | 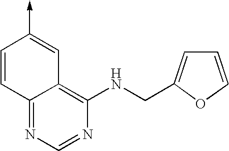 | 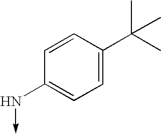 | | 500 |
| 296 | 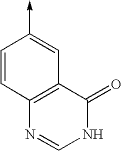 | 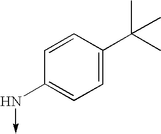 | | 421 |
| 297 | 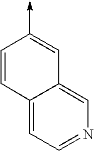 | 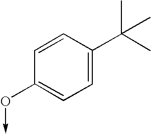 | | 405 |
| 298 | 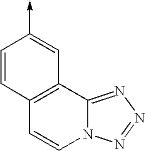 | 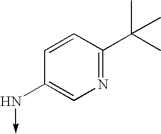 | | 445 |
| 299 | 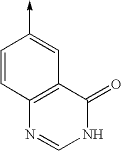 | 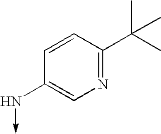 | | 422 |
| 300 | 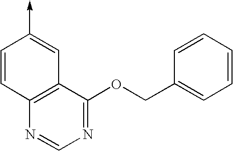 | 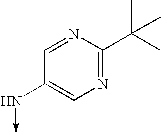 | | 513 |
| 301 | 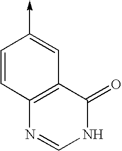 | 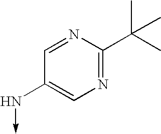 | | 423 |
| 302 | 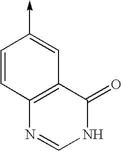 | 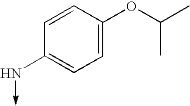 | | 423 |
| 303 | 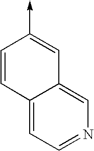 | 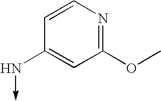 | | 379 |
| 304 | 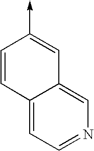 | 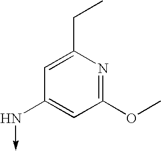 | | 407 |
| 305 | 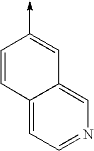 | 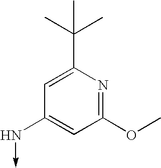 | | 435 |
| 306 | 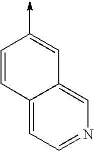 | 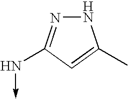 | | 352 |
| 307 | 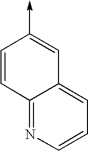 | 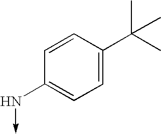 | | 405 |
| 308 | 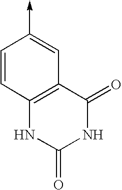 | 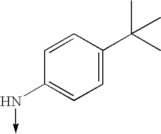 | | 438 |
| 309 | 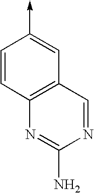 | 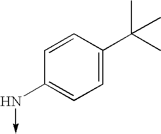 | | 421 |
| 310 | 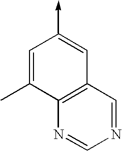 | 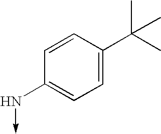 | | 420 |
| 311 | 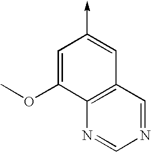 | 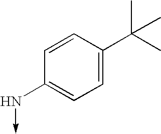 | | 436 |
| 312 | 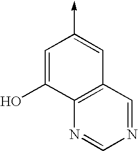 | 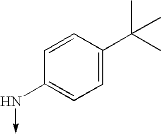 | | 421 |
| 313 | 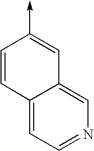 | 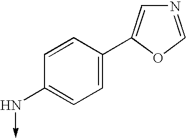 | | 415 |
| 314 | 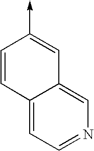 | 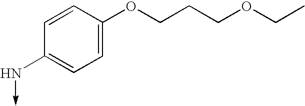 | | 450 |
| 315 | 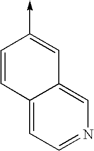 | 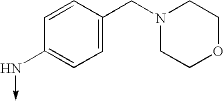 | | 447 |
| 316 | 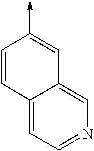 | 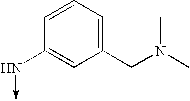 | | 405 |
| 317 | 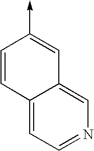 | 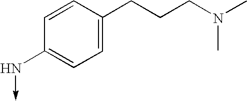 | | 433 |
| 318 | 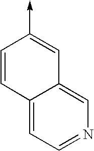 | 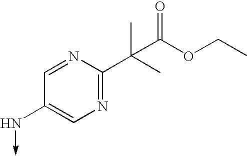 | | 464 |
| 319 | 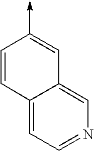 | 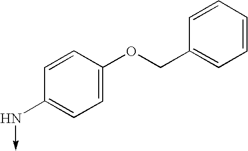 | | 454 |
| 320 | 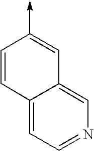 | 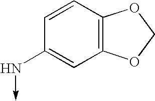 | | 392 |
| 321 | 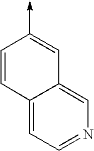 | 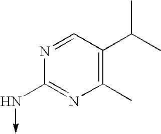 | | 406 |
| 322 | 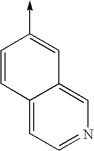 | 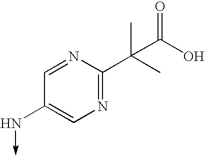 | | 436 |
| 323 | 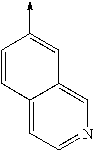 | 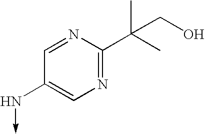 | | 422 |
| 324 | 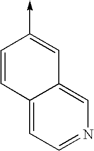 | 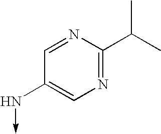 | | 392 |
| 325 | 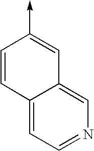 | 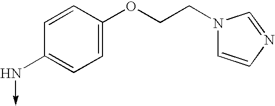 | | |
| 326 | 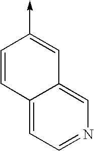 | 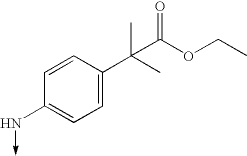 | | 462 |
| 327 | 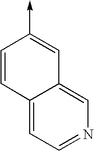 | 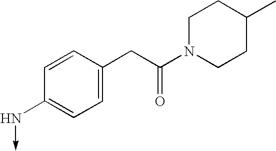 | | 487 |
| 328 | 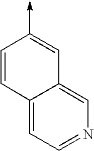 | 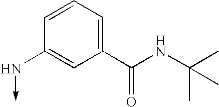 | | 447 |
| 329 | 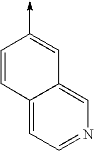 | 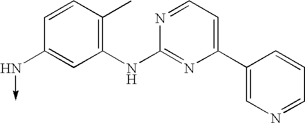 | | 532 |
| 330 | 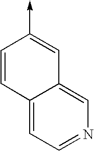 | 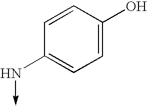 | | 364 |
| 331 | 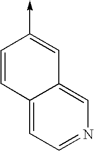 | 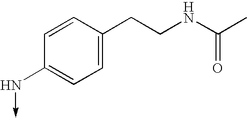 | | 446 |
| 332 | 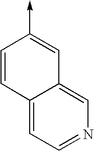 | 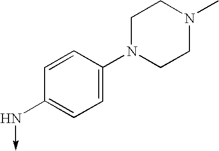 | | 446 |

TABLE 3

| | Structure | M + 1 |
|---|---|---|
| 1 | 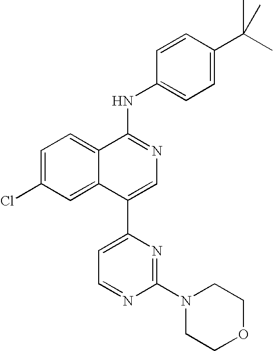 | 474 |
| 2 | 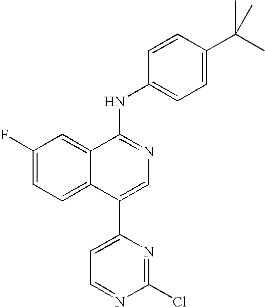 | 407.2 |
| 3 | 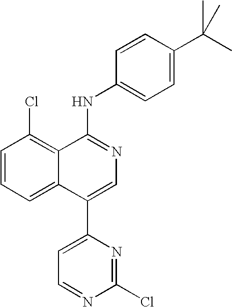 | 423 |
| 4 | 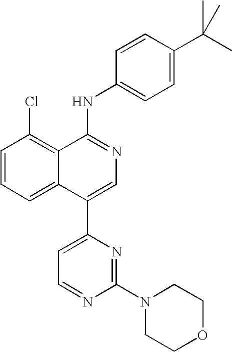 | 474 |
| 5 | 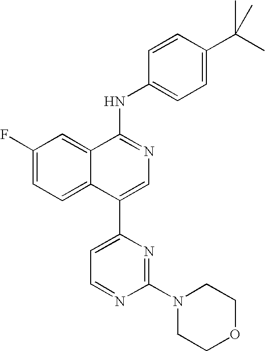 | 455.4 |

### EXAMPLE A

Detection of T1796A Mutation in the Human B-Raf Gene

| | |
|---|---|
| Detection Primer: | GATTTTGGTCTAGCTACAGA |
| Second Primer: | GACTTTCTAGTAACTCAGCAG |

Genomic DNA is isolated from human cells from a melanoma cell line using a GENELUTE mammalian genomic DNA kit (Sigma Cat. # G1N 350). PCR reactions are carried out on a PCR machine (MJ Research, Model PTC100) in a total volume of 50 mL using the PCR Core kit by Roche (Cat. # 1578 553). The PCR reaction mixture contains 5 mL of 10× reaction buffer, 1 mL of 10 mM dNTPs, 100-1000 ng of template DNA, 0.5 mL Taq polymerase (2.5-5 units), 1 mL of a 31 μM stock of each primer.

The PCR conditions are as follows:

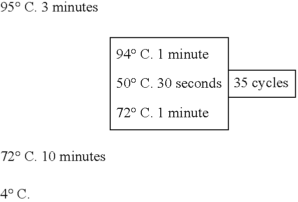

After amplification, 8 mL of the PCR reaction mixture is mixed with 2 mL of nucleic acid sample loading buffer (BioRad Cat. #161-0767). The 10 mL sample is loaded onto a 1.5% agarose (GIBCO-BRL Cat. # 15510-027) gel that contains 0.3 μg/mL of ethidium bromide (Pierce Cat. #17898). Molecular weight standards (100 bp DNA ladder from Invitrogen Cat. # 10380-012) are loaded in an adjacent lane. The DNA is separated by electrophoresis in TAE buffer (0.04 M tris-acetate, 0.01 M EDTA, 0.02 M glacial acetic acid pH 8.4) (Roche Cat. #1666690). Electrophoresis conditions are 120 volts for 30-40 minutes. After separation, the gel is exposed to UV light and a picture taken on an AlphaImager2000 documentation system.

Generally, two bands are detected in the gel. The faster migrating band runs ahead of the 100 bp marker and represents the primers. The DNA that results from the T1796A mutant specific PCR amplification has a predicted size of 152 bp and migrates between the 100 bp standard the 200 bp standard as predicted. The PCR amplification product is confirmed by sequencing. The presence of the PCR amplification product demonstrates that the T1796A mutation is present in the template DNA. The absence of the PCR amplification product is evidence that the mutation is absent in the tissue sample.

Other B-RAF mutations are detected by this method utilizing the detection primer and second primer indicated for the mutation in the following tables:

| | | |
|---|---|---|
| SEQ | | B-RAF |
| ID NO: | Oligonucleotide Segment (5′→3′) | Mutation |

Detection Primer

| | | |
|---|---|---|
| 1 | ACAGTGGGACAAAGAATTGA | G1388A |
| 2 | ACAGTGGGACAAAGAATTGT | G1388T |
| 3 | GGACAAAGAATTGGATCTGC | G1394C |
| 4 | GGACAAAGAATTGGATCTGA | G1394A |
| 5 | GGACAAAGAATTGGATCTGT | G1394T |
| 6 | ATTGGATCTGGATCATTTGC | G1403C |
| 7 | ATTGGATCTGGATCATTTGA | G1403A |
| 8 | GAGTAATAATATATTTCTTCATA | G1753A |
| 9 | CAGTAAAAATAGGTGATTG | T1782G |
| 10 | CAGTAAAAATAGGTGATTTTC | G1783C |
| 11 | GTAAAAATAGGTGATTTTGGTG | C1786G |
| 12 | GTAAAAATAGGTGATTTTGGTCG | T1787G |
| 13 | GATTTTGGTCTAGCTACAGA | T1796A |
| 14 | GATTTTGGTCTAGCTAGAGAT | TG1796-97AT |

Second Primer

| | | |
|---|---|---|
| 15 | TGTCACCACATTACATACTTACC | G1388A |
| 16 | TGTCACCACATTACATACTTACC | G1388T |
| 17 | TGTCACCACATTACATACTTACC | G13940 |
| 18 | TGTCACCACATTACATACTTACC | G1394A |
| 19 | TGTCACCACATTACATACTTACC | G1394T |
| 20 | TGTCACCACATTACATACTTACC | G1403C |
| 21 | TGTCACCACATTACATACTTACC | G1403A |
| 22 | GACTTTCTAGTAACTCAGCAG | G1753A |
| 23 | GACTTTCTAGTAACTCAGCAG | T1782G |
| 24 | GACTTTCTAGTAACTCAGCAG | G17830 |
| 25 | GACTTTCTAGTAACTCAGCAG | C1786G |
| 26 | GACTTTCTAGTAACTCAGCAG | T1787G |
| 27 | GACTTTCTAGTAACTCAGCAG | T1796A |
| 28 | GACTTTCTAGTAACTCAGCAG | TG1796-97AT |

1. A compound of formula (I) 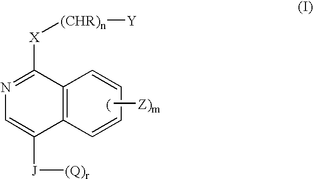
wherein
n is from 0-2;
r is from 0 to 2
m is from 0-4;
J is unsubstituted or substituted once or twice by Q, wherein
J is aryl, heteroaryl, cycloalkyl or heterocycloalkyl, wherein
aryl is an aromatic radical having from 6-14 carbon atoms, such as phenyl, naphthyl, fluorenyl and phenanthrenyl;
heteroaryl is an aromatic radical having from 4-14, especially from 5-7 ring atoms, of which 1, 2 or 3 atoms are chosen independently from N, S and O, such as furyl, pyranyl, pyridyl, 1,2-, 1,3- and 1,4-pyrimidinyl, pyrazinyl, triazinyl, triazolyl, oxazolyl, quinazolyl, imidazolyl, pyrrolyl, isoxazolyl isothiazolyl, indolyl, isoindolinyl, quinolyl, isoquinolyl, purinyl, cinnolinyl, naphthyridinyl, phthalazinyl, isobenzofuranyl, chromenyl, purinyl, thianthrenyl, xanthenyl, acridinyl, carbazolyl and phenazinyl;
cycloalkyl is a saturated cyclic radical having from 3-8, preferably from 5-6 ring atoms, such as cyclopropyl, cyclopentyl and cyclohexyl;
heterocycloalkyl is a saturated cyclic radical having from 3-8, preferably from 5-6 ring atoms, of which 1, 2 or 3 atoms are chosen independently from N, S and O, such as piperidyl, piperazinyl, imidazolidinyl, pyrrolidinyl and pyrazolidinyl;
Q is a substituent on 1 or 2 carbon atoms selected from the group consisting of halogen, unsubstituted or substituted lower alkyl, —OR₂, —SR₂, —N(R)R, —NRS(O)₂N(R)R, —NRS(O)₂R, —S(O)R₂, —S(O)₂R₂, —OCOR₂, —C(O)R₂, —CO₂R₂, —NR—COR₂, —CON(R₂)R₂, —S(O)₂N(R₂)R₂, cyano, tri-methylsilanyl, unsubstituted or substituted aryl, unsubstituted or substituted heteroaryl, such as substituted or unsubstituted imidazolyl, and substituted or unsubstituted pyridinyl, unsubstituted or substituted cycloalkyl, unsubstituted or substituted heterocycloalkyl, such as substituted or unsubstituted piperidinyl, substituted or unsubstituted piperazolyl, substituted or unsubstituted tetrahydropyranyl, and substituted or unsubstituted azetidinyl, —C₁₋₄alkyl-aryl, —C₁₋₄alkyl-heteroaryl, —C₁₋₄alkyl-heterocyclyl, amino, mono- or di-substituted amino, heteroaryl-aryl;
R is H, lower alkyl or lower alkoxy-alkyl;
R₂ is unsubstituted or substituted alkyl, unsubstituted or substituted cycloalkyl, unsubstituted or substituted phenyl, —C₁₋₄alkyl-aryl, —C₁₋₄alkyl-heteroaryl or —C₁₋₄alkyl-heterocycloalkyl;
X is a bond, Y, —N(R)—, oxa, thio, sulfone, sulfoxide, sulfonamide, amide, or ureylene, preferably —NH—, —NHC(O)—, —NHC(O)NH—;
Y is H, lower alkyl, substituted or unsubstituted aryl, substituted or unsubstituted heteroaryl, substituted or unsubstituted cycloalkyl or substituted or unsubstituted heterocycloalkyl; and
Z is amino, mono- or di-substituted amino, halogen, alkyl, substituted alkyl, hydroxy, etherified or esterified hydroxy, nitro, cyano, carboxy, esterified carboxy, alkanoyl, carbamoyl, N-mono- or N,N-di-substituted carbamoyl, amidino, guanidino, mercapto, sulfo, phenylthio, phenyl-lower alkylthio, alkylphenylthio, phenylsulfinyl, phenyl-lower alkylsulfinyl, alkylphenylsulfinyl, phenylsulfonyl, phenyl-lower alkanesulfonyl or alkylphenylsulfonyl, and where, if more than one radical Z is present (m≧2), the substituents Z are identical or different;
or an N-oxide of the mentioned compound, wherein one or more N atoms carry an oxygen atom;
or a pharmaceutically acceptable salt thereof.

2. A compound of formula (Ia) 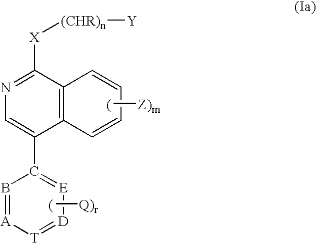
wherein
r is from 0-2;
n is from 0-2;
m is from 0-4;
A, B, D, E and T are each CH or CQ or
A, B, D and E are each CH or CQ and T is N or
B, D, E. and T are each CH or CQ and A is N or
A, B, T and E are each CH or CQ and D is N or
A, B, D, and T are each CH or CQ and E is N or
A, B and D are each CH or CQ and E and T are N or
B, E, and T are each CH or CQ and A and D are each N or
A, D and T are each CH or CQ and B and E are each N or
A and D are each CH or CQ and B, E and T are each N;
Q is a substituent on 1 or 2 carbon atoms selected from the group consisting of halogen, unsubstituted or substituted lower alkyl, —OR₂, —SR₂, —N(R)R, —NRS(O)₂N(R)R, —NRS(O)₂R, —S(O)R₂, —S(O)₂R₂, —OCOR₂, —C(O)R₂, —CO₂R₂, —NR—COR₂, —CON(R₂)R₂, —S(O)₂N(R₂)R₂, cyano, tri-methylsilanyl, unsubstituted or substituted aryl, unsubstituted or substituted heteroaryl, such as substituted or unsubstituted imidazolyl, and substituted or unsubstituted pyridinyl, unsubstituted or substituted cycloalkyl, unsubstituted or substituted heterocycloalkyl, such as substituted or unsubstituted piperidinyl, substituted or unsubstituted piperazolyl, substituted or unsubstituted tetrahydropyranyl, and substituted or unsubstituted azetidinyl, —C₁₋₄alkyl-aryl, —C₁₋₄alkyl-heteroaryl, —C₁₋₄alkyl-heterocyclyl, amino, mono- or di-substituted amino, heteroaryl-aryl;
R is H, lower alkyl or lower alkoxy-alkyl;
R₂ is unsubstituted or substituted alkyl, unsubstituted or substituted cycloalkyl, unsubstituted or substituted phenyl, —C₁₋₄alkyl-aryl, —C₁₋₄alkyl-heteroaryl or —C₁₋₄alkyl-heterocycloalkyl;
X is a bond, Y, —N(R)—, oxa, thio, sulfone, sulfoxide, sulfonamide, amide, or ureylene, preferably —NH—, —NHC(O)—, —NHC(O)NH—;
Y is H, lower alkyl, substituted or unsubstituted aryl, substituted or unsubstituted heteroaryl, substituted or unsubstituted cycloalkyl or substituted or unsubstituted heterocycloalkyl; and
Z is amino, mono- or di-substituted amino, halogen, alkyl, substituted alkyl, hydroxy, etherified or esterified hydroxy, nitro, cyano, carboxy, esterified carboxy, alkanoyl, carbamoyl, N-mono- or N,N-di-substituted carbamoyl, amidino, guanidino, mercapto, sulfo, phenylthio, phenyl-lower alkylthio, alkylphenylthio, phenylsulfinyl, phenyl-lower alkylsulfinyl, alkylphenylsulfinyl, phenylsulfonyl, phenyl-lower alkanesulfonyl or alkylphenylsulfonyl, and where, if more than one radical Z is present (m≧2), the substituents Z are identical or different;
or an N-oxide or a pharmaceutically acceptable salt thereof.

3. The compound of claim 2,
wherein
r is from 0-2;
n is 0 or 1;
m is 0 or 1;
A, B, D and E are each CH or CQ and T is N or
A, B, T and E are each CH or CQ and D is N or
A, B and D are each CH or CQ and E and T are each N;
Q is a substituent on one or two carbon atoms selected from the group consisting of halogen, unsubstituted or substituted lower alkyl, —OR₂, —SR₂, —NR₂, —NRS(O)₂N(R)₂, —NRS(O)₂R, —S(O)R₂, —S(O)₂R₂, —OCOR₂, —C(O)R₂, —CO₂R₂, —NR—COR₂, —CON(R₂)₂, —S(O)₂N(R₂)₂, cyano, tri-methylsilanyl, unsubstituted or substituted aryl, unsubstituted or substituted heteroaryl, unsubstituted or substituted cycloalkyl, unsubstituted or substituted heterocycloalkyl, —C₁₋₄alkyl-aryl, —C₁₋₄alkyl-heteroaryl, —C₁₋₄alkyl-heterocyclyl, amino, mono- or di-substituted amino;
R is H or lower alkyl,
R₂ is unsubstituted or substituted alkyl, unsubstituted or substituted cycloalkyl, phenyl, —C₁₋₄alkyl-aryl, —C₁₋₄alkyl-heteroaryl or —C₁₋₄alkyl-heterocycloalkyl;
X is —NR—, oxa or thia;
Y is phenyl that is unsubstituted or substituted by one or two identical or different substituents selected from the group consisting of amino; lower alkanoylamino, halogen, lower alkyl, halo-lower alkyl, hydroxy; lower alkoxy, phenyl-lower alkoxy, and cyano, or alternatively or additionally to the preceding group of substituents, lower alkenyl, C₈₋₁₂alkoxy, lower alkoxycarbonyl, carbamoyl, lower alkylcarbamoyl, lower alkanoyl, halo-lower alkyloxy, lower alkoxycarbonyl, lower alkylmercapto, halo-lower alkylmercapto, hydroxy-lower alkyl, lower alkanesulfonyl, halo-lower alkanesulfonyl, phenylsulfonyl, dihydroxybora (—B(OH)₂) and lower alkylenedioxy or
Y is pyridyl; and
Z is halogen, amino, N-lower alkylamino, hydroxy-lower alkylamino, phenyl-lower alkylamino, N,N-di-lower alkylamino, N-phenyl-lower alkyl-N-lower alkylamino, N,N-di-lower alkylphenylamino, lower alkanoylamino, or a substituent selected from the group consisting of benzoylamino and phenyl-lower alkoxycarbonylamino, wherein the phenyl radical in each case is unsubstituted or is substituted by nitro or by amino, or also by halogen, amino, N-lower alkylamino, N,N-di-lower alkylamino, hydroxy, cyano, carboxy, lower alkoxycarbonyl, lower alkanoyl or by carbamoyl;
or an N-oxide or a pharmaceutically acceptable salt thereof.

4. The compound of claim 3,
wherein
r is from 0-2;
n is 0 or 1;
m is 0 or 1;
A, B, D and E are each CH or CQ and T is N, or A, B and D are each CH or CQ and E and T are each N;
Q is bonded to A, to D or to A and D; and is selected from fluorine, chlorine or bromine, methyl, ethyl, propyl; hydroxy, methoxy, ethoxy, 2-hydroxyethoxy, 2-methoxyethoxy, (2-(1H-imidazol-1-yl)ethoxy, hydroxyiminomethyl, acetyl, formyl, methylmercapto, or amino, N-methylamino, N-ethylamino, N-(n)-propyl- or N-isopropylamino, 2-cyanoethylamino, 3-(methoxyphenyl)amino, 3-(4-morpholinyl)propylamino, 3-(pyridinyl)methylamino, 2-(2-pyridinyl)ethylamino, 4-(1H-imidazol-1-yl)butylamino, 4-(trifluoromethoxyphenyl)amino), (methylaminosulfonyl)amino, (methylsulfonyl)amino, (tetrahydro-2H-pyran-4-yl)amino, (tetrahydro-2H-pyran-4-yl)methylamino, (tetrahydro-3-furanyl)amino, (2-(1H-imidazol-1-yl)ethyl)amino, 2-hydroxyethylamino, (2-methoxyethyl)methylamino, 2-(2-hydroxyethoxy)ethylamino, spirans, 1-azetidinyl, 3-ethoxycarbonyl-1-azetidinyl, 3-carboxy-1-azetidinyl, tetrahydro-2H-1,3-oxazinyl, dihydro-1,2,5-oxathiazin-5(6H)-yl, tetrahydro-1(2H)-pyrimidinyl), 3-(acetyltetrahydro)-1(2H)-pyrimidinyl, piperazinyl, 4-(2-hydroxyethyl)-1-piperazinyl, 4-(ethoxycarbonyl)-1-piperazinyl, 4-acetyl-1-piperazinyl, piperidinyl, 4-(trifluormethyl)-1-piperidinyl, 4-(difluoromethyl)-1-piperidinyl, 4-(phenylmethyl)-1-piperidinyl, 4-phenoxy-1-piperidinyl, 4-cyano-1-piperidinyl, 4-methoxy-1-piperidinyl, 4-ethoxycarbonyl-1-piperidinyl, 4-hydroxy-1-piperidinyl, 4-carboxy-1-piperidinyl, 4-(aminocarbonyl)-1-piperidinyl, 4-methylthio-1-piperidinyl, 4-methylsulfonyl-1-piperidinyl, (tetrahydro-2H-pyran-4-yl)oxy, 4-morpholinyl, 3,5-dimethylmorpholinyl or 2-phenyl-4-morpholinyl;
R is H or methyl;
X is —NR—, oxa or thia;
Y is phenyl that is unsubstituted or substituted by one or two identical or different substituents selected from amino; acetylamino; fluorine, chlorine or bromine; tert-butyl, methyl, ethyl or propyl; trifluoromethyl; hydroxy; methoxy, ethoxy; benzyloxy; cyano, or (alternatively or additionally to the preceding group of substituents) ethenyl, C₈₋₁₂alkoxy, tert-butoxycarbonyl, carbamoyl, N-methyl-carbamoyl or N-tert-butyl-carbamoyl, acetyl, phenyloxy, trifluoromethoxy, 1,1,2,2-tetrafluoroethyloxy, ethoxycarbonyl, methylmercapto, trifluoromethylmercapto, hydroxymethyl, methanesulfonyl, trifluoromethanesulfonyl, phenylsulfonyl, dihydroxybora (—B(OH)₂), 2-methyl-pyrimidin-4-yl, oxazol-5-yl, 2-methyl-1,3-dioxolan-2-yl, 1H-pyrazol-3-yl, 1-methyl-pyrazol-3-yl, methylenedioxy, bonded to two adjacent carbon atoms or
Y is pyridyl, 2-, 3- or 4-aminophenyl, 2-, 3- or 4-acetylaminophenyl, 2-, 3- or 4-fluorophenyl, 2-, 3- or 4-chlorophenyl, 2-, 3- or 4-bromophenyl, 2,3-, 2,4-, 2,5- or 3,4-dichlorophenyl, chloro-fluoro-phenyl, 4-chloro-2-fluoroanilino, 2-, 3- or 4-methylphenyl, 2-, 3- or 4-ethylphenyl, 2-, 3- or 4-propylphenyl, methyl-fluoro-phenyl, 2-, 3- or 4-trifluoromethylphenyl, 2-, 3- or 4-hydroxyphenyl, 2-, 3- or 4-methoxyphenyl, 2-, 3- or 4-ethoxyphenyl, methoxy-chloro-phenyl, 2-, 3- or 4-benzyloxyphenyl, 2-, 3- or 4-cyanophenyl, 2-, 3- or 4-methylphenyl, 4-chloro-5-trifluoromethylphenyl, 3-bromo-5-trifluoromethylphenyl, 3,5-dimethylphenyl, 4-methyl-3-iodophenyl, 3,4-bis(trifluoromethyl)phenyl, 3-bromo-4-ethyl-phenyl or 3-chlorobenzylphenyl; and
Z is halogen, amino, N-lower alkylamino, hydroxy-lower alkylamino, phenyl-lower alkylamino, N,N-di-lower alkylamino, N-phenyl-lower alkyl-N-lower alkylamino; N,N-di-lower alkylphenylamino, lower alkanoylamino or a substituent selected from the group consisting of benzoylamino and phenyl-lower alkoxycarbonylamino, wherein the phenyl radical in each case is unsubstituted or, is substituted by nitro or by amino, or also by halogen, amino, N-lower alkylamino, N,N-di-lower alkylamino, hydroxy, cyano, carboxy, lower alkoxycarbonyl, lower alkanoyl or by carbamoyl;
or an N-oxide or a pharmaceutically acceptable salt thereof.

5. The compound of claim 2,
wherein
r is 1;
n is 0;
m is 0;
B, D, E, and T are CH or CQ and A is N or
A, B, D and E are each CH or CQ and T is N;
Q is a substituent on one or two carbon atoms selected from fluorine, chlorine, methyl, ethyl, propyl; amino, N-methylamino, N-ethylamino, N-(n)-propylamino, N-isopropylamino, 2-cyanoethylamino, 3-(methoxyphenyl)amino, 3-(4-morpholinyl)propylamino, 3-(pyridinyl)methylamino, 2-(2-pyridinyl)ethylamino, 4-(1H-imidazol-1-yl)butylamino, 4-(trifluoromethoxyphenyl)amino), (methylaminosulfonyl)amino, (methylsulfonyl)amino, (tetrahydro-2H-pyran-4-yl)amino, (tetrahydro-2H-pyran-4-yl)methylamino, (tetrahydro-3-furanyl)amino, (2-(1H-imidazol-1-yl)ethyl)amino, 2-hydroxyethylamino, 2-(2-hydroxyethoxy)ethylamino, tetrahydro-1(2H)-pyrimidinyl), 3-(acetyltetrahydro)-1(2H)-pyrimidinyl, piperazinyl, 4-(2-hydroxyethyl)-1-piperazinyl, 4-(ethoxycarbonyl)-1-piperazinyl, 4-acetyl-1-piperazinyl, piperidinyl, 4-(trifluormethyl)-1-piperidinyl, 4-(difluoromethyl)-1-piperidinyl, 4-(phenylmethyl)-1-piperidinyl, 4-phenoxy-1-piperidinyl, 4-cyano-1-piperidinyl, 4-methoxy-1-piperidinyl, 4-ethoxycarbonyl-1-piperidinyl, 4-hydroxy-1-piperidinyl, 4-carboxy-1-piperidinyl, 4-(aminocarbonyl)-1-piperidinyl, 4-methylthio-1-piperidinyl, 4-methylsulfonyl-1-piperidinyl, 4-morpholinyl, 3,5-dimethylmorpholinyl or 2-phenyl-4-morpholinyl;
R is H or methyl,
X is —NH—; and
Y is phenyl that is unsubstituted or substituted by one or two identical or different substituents selected from fluorine, chlorine, bromine; lower alkyl, trifluoromethyl; 4-chlorophenyl, 2-, 3- or 4-methylphenyl, 4-chloro-5-trifluoromethylphenyl, 3-bromo-5-trifluoromethylphenyl, 3,5-dimethylphenyl; 4-methyl-3-iodophenyl, 3,4-bis(trifluoromethyl)phenyl or 3-bromo-4-ethyl-phenyl;
or an N-oxide or pharmaceutically acceptable salt thereof.

6. The compound of claim 2,
wherein
r is 1;
n is 0;
m is 0;
A, B, D and E are each CH or CQ and T is N;
Q is a substituent on one carbon atom selected from amino, N-methylamino, N-ethylamino, N-(n)-propylamino, N-isopropylamino, 2-cyanoethylamino, 3-(methoxyphenyl)amino, 3-(4-morpholinyl)propylamino, 3-(pyridinyl)methylamino, 2-(2-pyridinyl)ethylamino, 4-(1H-imidazol-1-yl)butylamino, 4-(trifluoromethoxyphenyl)amino), (methylaminosulfonyl)amino, (methylsulfonyl)amino, (tetrahydro-2H-pyran-4-yl)amino, (tetrahydro-2H-pyran-4-yl)methylamino, (tetrahydro-3-furanyl)amino, (2-(1H-imidazol-1-yl)ethyl)amino, 2-hydroxyethylamino, 2-(2-hydroxyethoxy)ethylamino, piperidinyl, 4-(trifluormethyl)-1-piperidinyl, 4-(difluoromethyl)-1-piperidinyl, 4-(phenylmethyl)-1-piperidinyl, 4-phenoxy-1-piperidinyl, 4-cyano-1-piperidinyl, 4-methoxy-1-piperidinyl, 4-ethoxycarbonyl-1-piperidinyl, 4-hydroxy-1-piperidinyl, 4-carboxy-1-piperidinyl, 4-(aminocarbonyl)-1-piperidinyl, 4-methylthio-1-piperidinyl, 4-methylsulfonyl-1-piperidinyl or morpholinyl;
R is H;
X —NH—; and
Y is phenyl that is unsubstituted or substituted by chlorine, methyl, trifluoromethyl, isopropyl, tert-butyl, methoxy, 4-trifluoromethoxyphenyl; naphthyl; cyclohexyl that is unsubstituted or substituted by lower alkyl, indolyl that is unsubstituted or substituted by halogen or by lower alkyl;
or an N-oxide or pharmaceutically acceptable salt thereof.

7. The compound of claim 6,
wherein
r is 1;
n is 0;
m is 0;
A, B, D, and E are each CH and T is N;
Q is a substituent on one carbon atom selected from morpholinyl;
R is H;
X is —NH—; and
Y is phenyl that is substituted in the 4-position by tert-butyl or trifluoromethyl;
or an N-oxide or pharmaceutically acceptable salt thereof.

8. The compound of claim 4,
wherein
r is 1;
n is 0;
m is 0;
A, B and D are each CH, and E and T are each N;
X is —NH—;
Y is phenyl that is substituted in the 4-position by tert-butyl; and
Q is a 2-hydroxyethylamino substituent on D;
or an N-oxide or pharmaceutically acceptable salt thereof.

9. The compound of claim 1,
wherein
n is from 0-2;
r is from 0-2;
m is from 0-4;
J is a bicyclic heteroaromatic ring system, selected from indolyl, isoindolinyl, quinolyl, isoquinolyl, quinazolyl, purinyl, cinnolinyl, naphthyridinyl, phthalazinyl, isobenzofuranyl naphthyridinyl, phthalazinyl, chromenyl and purinyl;
Q is a substituent on either one or both rings of the bicyclic ring system, and on one or two carbon atoms on either one or both rings of the bicyclic ring system, selected from the group consisting of halogen, unsubstituted or substituted lower alkyl, —OR₂, —SR₂, —NR₂, —NRS(O)₂N(R)₂, —NRS(O)₂R, —S(O)R₂, —S(O)₂R₂, —OCOR₂, —C(O)R₂, —CO₂R₂, —NR—COR₂, —CON(R₂)₂, —S(O)₂N(R₂)₂, cyano, tri-methylsilanyl, unsubstituted or substituted aryl, unsubstituted or substituted heteroaryl, unsubstituted or substituted cycloalkyl, unsubstituted or substituted heterocycloalkyl, —C₁₋₄alkyl-aryl, —C₁₋₄alkyl-heteroaryl, —C₁₋₄alkyl-heterocyclyl, amino, mono- or di-substituted amino;
R is H or lower alkyl;
R₂ is unsubstituted or substituted alkyl, unsubstituted or substituted cycloalkyl, phenyl, —C₁₋₄alkyl-aryl, —C₁₋₄alkyl-heteroaryl or —C₁₋₄alkyl-heterocycloalkyl;
X is Y, —N(R)—, oxa, thio, sulfone, sulfoxide, sulfonamide, amide or ureylene;
Y is H, lower alkyl, substituted or unsubstituted aryl, substituted or unsubstituted heteroaryl, substituted or unsubstituted cycloalkyl or substituted or unsubstituted heterocycloalkyl; and
Z is amino, mono- or di-substituted amino, halogen, alkyl, substituted alkyl, hydroxy, etherified or esterified hydroxy, nitro, cyano, carboxy, esterified carboxy, alkanoyl, carbamoyl, N-mono- or N,N-di-substituted carbamoyl, amidino, guanidino, mercapto, sulfo, phenylthio, phenyl-lower alkylthio, alkylphenylthio, phenylsulfinyl, phenyl-lower alkylsulfinyl, alkylphenylsulfinyl, phenylsulfonyl, phenyl-lower alkanesulfonyl or alkylphenylsulfonyl, and where, if more than one radical Z is present (m≧2), the substituents Z are identical or different;
or an N-oxide or a pharmaceutically acceptable salt thereof.

10. The compound of claim 9,
wherein
n is 0;
r is 0;
m is 0;
J is a bicyclic heteroaromatic ring system, selected from indolyl, isoindolinyl, quinolyl, isoquinolyl, quinazolyl, purinyl, cinnolinyl, naphthyridinyl, phthalazinyl, isobenzofuranyl naphthyridinyl, phthalazinyl, chromenyl and purinyl;
R is H or lower alkyl;
X is Y, —N(R)—, oxa, thio, sulfone, sulfoxide, sulfonamide, amide or ureylene; and
Y is H, lower alkyl, substituted or unsubstituted aryl, substituted or unsubstituted heteroaryl, substituted or unsubstituted cycloalkyl or substituted or unsubstituted heterocycloalkyl;
or an N-oxide or a pharmaceutically acceptable salt thereof.

11. The compound of claim 10,
wherein
n is 0;
r is 0;
m is 0;
J is isoquinolyl;
X is NH; and
Y is 4-tert-butylphenyl;
or an N-oxide or a pharmaceutically acceptable salt thereof.

12. The compound of claim 10,
wherein
n is 0;
r is 0;
m is 0;
J is quinazolyl;
X is NH; and
Y is 4-tert-butylphenyl;
or an N-oxide or a pharmaceutically acceptable salt thereof.

13. The compound of claim 10,
wherein
n is 0;
r is 0;
m is 0;
J is isoquinolyl;
X is NH; and
Y is 2-tert-butyl-pyrimidin-5-yl;
or an N-oxide or a pharmaceutically acceptable salt thereof.

14. A pharmaceutical composition comprising a compound according to claim 1 in combination with a pharmaceutically acceptable carrier.

15. A method of treating a patient having a disease characterized by excessive signaling through the MAP kinase signaling pathway, which comprises administering to the patient an effective RAF kinase inhibiting amount of a compound of claim 1.

16. The method of claim 15, wherein the disease characterized by excessive signaling through the MAP kinase signaling pathway is a cancer.

17. The method of claim 16, wherein the cancer is a melanoma, a colorectal cancer, an ovarian cancer, a glioma, an adenocarcinoma, a sarcoma, a breast cancer or a liver cancer.

18. The method of claim 17, wherein the cancer is a melanoma.

19. A method of treating melanoma in a patient which comprises:
(a) testing melanoma tissue from the patient to determine whether the melanoma tissue expresses mutant RAF kinase or overexpresses a wild-type RAF kinase; and
(b) treating the patient if the melanoma tissue is found to overexpress a wild-type RAF kinase or express an activating mutant B-RAF kinase with an effective RAF kinase inhibiting amount of a compound of claim 1.

20. The method of claim 19, wherein the mutant RAF kinase corresponds to a mutation in the B-RAF kinase gene selected from G1388A, G1388T, G1394C, G1394A, G1394T, G1403C, G1403A, G1753A, T1782G, G1783C, C1786G, T1787G, T1796A and TG1796-97AT.

21. The method of claim 20, wherein the melanoma expresses a mutant RAF kinase.

22. The method of claim 21, wherein the mutant RAF kinase is a V599E mutation.
